SEQUENCE LISTING (23 sequences)

SEQ ID NO: 1 - protein, 598 aa, Artificial Sequence
  Synthetic polypeptide
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 2 - protein, 528 aa, Artificial Sequence
  Synthetic polypeptide
MPTPDATTPQAKGFRRAVSELDAKQAEAIMVRGQGAPGPSLTGSPWPGTAAPAASYTPTPRSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 3 - protein, 443 aa, Artificial Sequence
  Synthetic polypeptide
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 4 - protein, 514 aa, Artificial Sequence
  Synthetic polypeptide
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 5 - DNA, 21 bp, Artificial Sequence
  Synthetic polynucleotide
tgatattacctccaaatgcca

SEQ ID NO: 6 - DNA, 17 bp, Artificial Sequence
  Synthetic polynucleotide
ggttcaccgaaaggtca

SEQ ID NO: 7 - protein, 262 aa, Artificial Sequence
  Synthetic polypeptide
MKKGHHHHHHGFEIDKVWYDLDAGAISLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 8 - protein, 276 aa, Artificial Sequence
  Synthetic polypeptide
MKKGHHHHHHGSGSENLYFQSGSGSGFEIDKVWYDLDAGSGSDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 9 - DNA, 19 bp, Artificial Sequence
  Synthetic polynucleotide
tgggaggccatcacattgt

SEQ ID NO: 10 - DNA, 21 bp, Artificial Sequence
  Synthetic polynucleotide
aatccagcaggtcagcaaaga

SEQ ID NO: 11 - DNA, 18 bp, Artificial Sequence
  Synthetic polynucleotide
caactacagcacaggcta

SEQ ID NO: 12 - DNA, 26 bp, Artificial Sequence
  Synthetic polynucleotide
gcatctgaatgtcttctaccttaatg

SEQ ID NO: 13 - DNA, 20 bp, Artificial Sequence
  Synthetic polynucleotide
tccaacctttcctggcccag

SEQ ID NO: 14 - DNA, 20 bp, Artificial Sequence
  Synthetic polynucleotide
gcatgaagggcaggaggaat

SEQ ID NO: 15 - DNA, 23 bp, Artificial Sequence
  Synthetic polynucleotide
gaagtccacctgctaaggaagaa

SEQ ID NO: 16 - DNA, 23 bp, Artificial Sequence
  Synthetic polynucleotide
tcactggagacacatgtacacag

SEQ ID NO: 17 - RNA, 21 nt, Artificial Sequence
  Synthetic polynucleotide
  misc_feature (20)..(21): deoxythymidine
gaaucagcuuucuuagaauuu

SEQ ID NO: 18 - RNA, 21 nt, Artificial Sequence
  Synthetic polynucleotide
  misc_feature (20)..(21): deoxythymidine
auucuaagaaagcugauucuu

SEQ ID NO: 19 - RNA, 21 nt, Artificial Sequence
  Synthetic polynucleotide
  misc_feature (20)..(21): deoxythymidine
gcaucgcaguugcuugacauu

SEQ ID NO: 20 - RNA, 21 nt, Artificial Sequence
  Synthetic polynucleotide
  misc_feature (21)..(21): deoxythymidine
ugucaagcaacugcgaugcgu

SEQ ID NO: 21 - RNA, 25 nt, Artificial Sequence
  Synthetic polynucleotide
  misc_feature (25)..(25): deoxythymidine
cuagguugaagauguuauaggcacu

SEQ ID NO: 22 - RNA, 27 nt, Artificial Sequence
  Synthetic polynucleotide
agugccuauaacaucuucaaccuagaa

SEQ ID NO: 23 - DNA, 28 bp, Artificial Sequence
  Synthetic polynucleotide
gatcctcgtgcgaaaaggtcaagcgcta

(19) United States
(12) Patent Application Publication
(10) Pub. No.: US 2023/0255934 A1
(43) Pub. Date: Aug. 17, 2023

(54) NURR1 RECEPTOR MODULATORS AND USES THEREOF

(71) Applicant: The Regents of the University of California, Oakland, CA (US)

(72) Inventors: Pamela M. England, Pacifica, CA (US); Matthew P. Jacobson, San Francisco, CA (US)

(21) Appl. No.: 17/919,376

(22) PCT Filed: Apr. 23, 2021
(86) PCT No.: PCT/US21/28799

§ 371 Date: Oct. 17, 2022

Related U.S. Application Data
(60) Provisional application No. 63/015,302, filed on Apr. 24, 2020.

(51) Int. Cl. A61K 31/404

(52) U.S. Cl. CPC A61K 31/404

(57) ABSTRACT

Described herein, inter alia, are Nurr1 receptor modulators and uses thereof.

### CROSS-REFERENCES TO RELATED APPLICATIONS

This application claims the benefit of U.S. Provisional Application No. 63/015,302, filed Apr. 24, 2020, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-681001WO_Sequence_Listing_ST25, created Apr. 15, 2021, 27,033 bytes, machine format IBM-PC, MS Windows operating system, is hereby incorporated by reference.

### STATEMENT AS TO RIGHTS TO INVENTIONS MADE UNDER FEDERALLY SPONSORED RESEARCH AND DEVELOPMENT

This invention was made with government support under grant no. R01 NS108404 awarded by the National Institutes of Health. The government has certain rights in the invention.

### BACKGROUND

Over one million Americans are currently living with Parkinson's disease (PD), and approximately 60,000 new cases are diagnosed each year. PD is the second most common degenerative neurological disorder, after Alzheimer's disease. Current PD therapeutics are symptom-modifying only, having no effect on disease progression, and lose efficacy over time. New therapeutic strategies are needed to combat this disease. The nuclear receptor Nurr1 μlays a critical role in the development, maintenance, and survival of midbrain dopaminergic neurons. PD is a neurodegenerative disorder characterized by the loss of midbrain dopaminergic neurons. Nurr1 modulators (e.g., agonists or inhibitors) may provide an orthogonal approach to increasing dopamine levels in the brain (management of symptoms), improving the health and preventing the degeneration of existing dopamine neurons (management of disease progression). Disclosed herein, inter alia, are solutions to these and other problems in the art.

### BRIEF SUMMARY

In an aspect is provided a compound having the formula

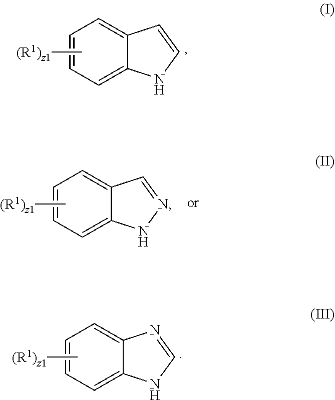

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SF₅, —SSR^{1D}, —SiR^{1A}R^{1B}R^{1C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, and R^{1D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl, or substituted or unsubstituted heteroaryl.

The variable n1 is independently an integer from 0 to 4.

The variables m1 and v1 are independently 1 or 2.

X¹ is independently —F, —Cl, —Br, or —I.

The variable z1 is an integer from 0 to 6.

In an aspect is provided a pharmaceutical composition including a compound described herein, or a pharmaceutically acceptable salt thereof, and a pharmaceutically acceptable excipient.

In an aspect is provided a method of treating a disease associated with dysregulation and/or degeneration of dopaminergic neurons in the central nervous system of a subject in need thereof, the method including administering to the subject in need thereof a therapeutically effective amount of a compound described herein, or a pharmaceutically acceptable salt thereof.

In an aspect is provided a method of treating a neurodegenerative disease in a subject in need thereof, the method including administering to the subject in need thereof a therapeutically effective amount of a compound described herein, or a pharmaceutically acceptable salt thereof.

In an aspect is provided a method of treating a cancer in a subject in need thereof, the method including administering to the subject in need thereof a therapeutically effective amount of a compound described herein, or a pharmaceutically acceptable salt thereof.

In an aspect is provided a method of reducing inflammation in a subject in need thereof, the method including administering to the subject in need thereof a therapeutically effective amount of a compound described herein, or a pharmaceutically acceptable salt thereof.

In an aspect is provided a method of reducing oxidative stress in a subject in need thereof, the method including administering to the subject in need thereof a therapeutically effective amount of a compound described herein, or a pharmaceutically acceptable salt thereof.

In an aspect is provided a method of modulating the level of activity of Nurr1 in a subject in need thereof, the method including administering to the subject in need thereof a therapeutically effective amount of a compound described herein, or a pharmaceutically acceptable salt thereof.

In an aspect is provided a method of differentiating a stem cell, the method including contacting the stem cell in vitro with a compound described herein, or a pharmaceutically acceptable salt thereof.

### BRIEF DESCRIPTION OF THE DRAWINGS

FIG. 1. The DHI analogs 5-chloroindole and 5-bromoindole bind directly to and stimulate the transcriptional activity of Nurr1. The electrostatic potential surface (EPS) of each DHI analog was calculated using the 6-31G** basis sets and the B3LYP-D3 functional in water (PBS solvent model). Binding affinity (K_{D}) for the Nurr1 ligand binding domain was determined using microscale thermophoresis. The Hill coefficient (n) for 5-chloroindole and 5-bromoindole is two, and for all other compounds is one. Relative expression of Nurr1's target genes Th and Vmat2 was determined using qPCR analysis of mRNA from MN9D cells following treatment (24 h) with compound (10 μM). Transcript levels for each target gene was normalized to the housekeeping gene Hprt, and to expression level for vehicle (DMSO only) treated cells. All experimental values are the result of three or more independent measurements±SD. *Note: a subset of the indoles tested showed signs of instability and polymerization in solution, as well as cytotoxicity in MN9D cells. In particular, acquisition of binding data is precluded by initial fluorescence quenching and photobleaching by the compounds (5/6-amino indoles) and autooxidation and subsequent polymerization of the compounds in solution (5/6-hydroxy and 5/6-amino indoles).

FIGS. 2A-2D. Point mutations (Arg563, His516) within the DHI binding pocket significantly impact the binding of 5-chloroindole and 5-bromoindole and point to a second indole binding site with the Nurr1 LBD. FIGS. 2A-2B: Single and double mutants increase the affinity of the 5-substituted indoles for the receptor and dramatically alter the thermophoresis response amplitude. FIGS. 2C-2D: Single and double mutants decrease the affinity of the 5,6-disubstituted indoles for Nurr1, but have relatively small effects on the thermophoresis response amplitude.

FIGS. 3A-3B. The binding of IQ and DHI to the Nurr1 LBD within the “566 site” is supported by a network molecular interactions. Close-up view of Nurr1 bound (FIG. 3A) covalently to indolequinone in the crystal structure (PDB:6DDA), and (FIG. 3B) non-covalently in a computational model of the unoxidized indole, DHI. In the QM/MM model, DHI is positioned farther away from H10/11 than the IQ, resulting in a new interaction with His516, and is tilted ˜45 degrees along the plane of the indole ring relative to the IQ, resulting in closer interactions with Glu445 and Arg563. In the apo LBD structure (PDB:1OVL), the guanidinium side chain of Arg563 is rotated ˜180 degrees and forms an intramolecular bond with the carboxylate side chain of Glu445 (not shown).

FIGS. 4A-4C. FIG. 4A: The binding of indoles to the Nurr1 LBD is stabilized by networks of hydrogen, halogen, cation-π, and ionic bonds. Top: Chemical structures showing interactions between amino acid side chains within the Nurr1 LBD and bound ligands; only interactions with distances ≤3.0 Å are shown. Bottom: Table showing the physical distances in A between amino acid side chains the bound ligands. Distances ≤3.0 Å are shown in black and distances >3 Å are shown in grey. FIG. 4B: Substituted indoles are predicted to bind with nearly identical poses to Nurr1 in computational (QM/MM) models. Top: Model of 5-chloroindole bound to Nurr1. Bottom: Overlay of computational models for all of the halogenated, and 5-substituted, indoles evaluated in the present study. FIG. 4C: Binding of 5-bromo- and 5-chloroindole to Nurr1 is predicted to be stabilized by a halogen bond with His516. Lateral views of the molecular ESP surfaces for the 5-halogen-substituted indoles highlight the interaction between the lone pair of electrons on His516 and the sigma hole within the bromo and chloro substituents. The deficiency in electron density in the outer lobe of the pz orbital of 5-bromoindole and 5-chloroindole results in a relatively more positive electrostatic potential surface in this region, compared to 5-fluoroindole. The relative pKa values, interaction energies, and measured binding affinities are consistent with the proposed halogen bond between His516 and a subset of the halogenated indoles. The pKa values were predicted using propKa 3.1 after QM/MM optimization of the non-covalently bound indoles. The single point interaction energies were calculated with the LMP2/cc-pVDZ** level of theory in the gas phase. The coordinates of the complexes were taken from the QM/MM optimized structures at the DFT-D3/LACVP* level of theory. Ranking is among all of the 5-substituted indoles in the present study.

FIG. 5. The DHI analogs 5-chloroindole and 5-bromoindole bind directly to stimulate the transcriptional activity of Nurr1. The molecular electrostatic potential (ESP) surface of each DHI analog was calculated using the 6-31G** basis sets, and bromine atoms treated with the LAV2P**. Binding affinity (K_{D}) for the Nurr1 LBD was determined using microscale thermophoresis. Relative expression levels of the Nurr1 target genes Th and Vmat2 were determined using qPCR analyses of mRNA isolated from MN9D cells following treatment with each compound (10 μM, 24 h). Transcript levels for each target gene were normalized to the housekeeping gene Hprt, and reported as the fold-change relative to cells treated with vehicle (DMSO) only. All experimental values are the result of three or more independent measurements±SD. Detailed experimental protocols are described in Example 3. Note: data acquisition for a subset of these compounds is precluded by their chemical instability. In particular, robust binding data could not be obtained because of initial fluorescence quenching and photobleaching (5- and 6-aminoindole), and autooxidation and polymerization in solution (5- and 6-hydroxyindole, 5- and 6-aminoindole). These compounds also exhibited significant cytotoxicity (see FIG. 6C).

FIGS. 6A-6C. A subset of the halogenated indoles bind to the Nurr1 ligand binding domain. Microscale thermophoresis (MST) binding isotherms for (FIG. 6A) 5-substituted, (FIG. 6B) 6-substituted, and (FIG. 6C) 5,6-dihalogenated indoles and the Nurr1 LBD are obtained by plotting the change in thermophoresis (Fₙ-Fₙ₀) versus the concentration of the compound tested ([Indole], M). All experimental values are the result of three or more independent measurements±SD. All data were best fit to a single site, except for 5-chloro and 5-bromoindole, which required used of the Hill equation. Note: The Hill coefficient (n_{H}) for 5-chloroindole (1.9±0.2) and 5-bromoindole (1.9±0.3) are both >1, whereas the value for all other compounds is unity within the error. A Hill coefficient greater than one typically indicates cooperative binding of ligands, with the absolute value setting the lower limit for the number of interacting binding sites (see Weiss, J. N. The Hill equation revisited: uses and misuses, FASEB J. 11, 835-841, 1997). However, we observed a significant change in the Hill coefficient with increasing concentrations of surfactant for 5-chloroindole, possibly due to partial denaturation of the protein and concomitant loss of one of the indole binding sites. Alternatively, increasing concentrations of surfactant may have broken up compound nano-aggregates that falsely signaled cooperative binding of two indoles.

FIGS. 7A-7D. Only a subset of the indoles that bind to Nurr1 also stimulate the transcription of Nurr1 target genes in MN9D cells. The effect of (FIG. 7A) 5-substituted, (FIG. 7B) 6-substituted, and (FIG. 7C) 5,6-dihalogenated indoles (10 μM, 24 h) relative to vehicle (DMSO) only (dashed line) on the expression of Nurr1, Th, and Vmat2 was quantified by qPCR as described in Example 3. FIG. 7D: The effect of 5-chloroindole on the expression of Th and Vmat2 is concentration dependent. All data are the result of three or more independent measurements and are expressed as an average±standard deviation (SD), with *p<0.05, **p<0.01, ***p<0.001, ****p<0.0001 by one-way ANOVA, in comparison with the response for vehicle treatment (DMSO).

FIGS. 8A-8B. 5-chloroindole is not cytotoxic. FIG. 8A: Approximately half of the indoles tested reduce the percentage of viable of MN9D cells following treatment with 10 μM compound for 24 h. FIG. 8B: 5-chloroindole has no significant effect on cell viability at concentrations ≤10 μM following treatment for 24 h. Cell viability was measured using CytoTox-Glo Cytotoxicity Assay Kit (Promega) according to the manufacturer's instructions following treatment of cells (10,000 cells/well) with the indicated indole or DMSO. All experimental values are the result of three independent measurements±SD, with *p<0.05, **p<0.01, ***p<0.001, ****p<0.0001 by one-way ANOVA, in comparison with the response for vehicle treatment (DMSO).

FIGS. 9A-9B. Increasing concentrations of surfactant decrease the formation of 5-chloroindole nano-aggregates and increase the affinity for the Nurr1 LBD by less than two-fold. FIG. 9A: Aggregate count (DLS normalized intensity) for 5-chloroindole measured with increasing percentages of Pluronic F127. FIG. 9B: Binding affinity of 5-chloroindole measured with increasing percentages of Pluronic F127; K_{D} (0.1%)=15.0±1.2 μM, (nH=2); K_{D} (0.2%)=8.3±0.7 μM, (nH=2); K_{D} (0.5%)=10.9±0.3 μM, (nH=1); K_{D} (1.0%)=9.1±0.4 μM, (n_{H}=1). All experimental values are the result of three or more independent biological replicates±standard deviation.

FIGS. 10A-10B. The DHI analog 5-chloroindole stimulates Nurr1 activity in two different luciferase reporter assays. In both the (FIG. 10A) Nurr1-LBD_Gal4-DBD luciferase reporter assay and the (FIG. 10B) full-length Nurr1 NBRE luciferase reporter assay, 5-chloroindole stimulates production of luciferase. Control compounds 5-cyanoindole (negative control) and amodiaquine (positive control) perform as expected. MN9D cells were individually treated with the indicated concentrations of ligands for 6 h prior to measuring luciferase signal (RLU, relative luminometer units; see Example 3 for additional details). All experimental values are the result of three or more independent biological replicates and are expressed as the relative average response±standard deviation, with *p<0.05, **p<0.01, ***p<0.001, ****p<0.0001 by one-way ANOVA, in comparison to the response with vehicle (DMSO) only.

FIGS. 11A-11C. The effect of 5-chloroindole on the expression of Nurr1 target genes depends on the expression of Nurr1. Expression of Nurr1, Th and Vmat2 transcripts was determined in the presence of 5-chloroindole (10 μM, 24 h) with (Nurr1 siRNA) or without (Ctrl siRNA) knockdown of Nurr1 levels. Gene expression levels in the presence of 5-chloroindole are relative to the same treatments with vehicle (DMSO) only. FIG. 11A: The expression of Nurr1 is significantly reduced by Nurr1 siRNA, but not control siRNA. Knockdown of Nurr1 in MN9D cells expressing endogenous Nurr1 with Nurr1 siRNA was carried out as described in Example 3. FIGS. 11B-11C: The effect of 5-chloroindole on the expression of Th and Vmat2 is significantly reduced in the presence of Nurr1 siRNA, but not control siRNA. All experimental values are the result of three or more independent biological replicates and are expressed as the relative average response±standard deviation, with * p<0.05, **p<0.01, ***p<0.001, ****p<0.0001 by one-way ANOVA, in comparison to the response with vehicle (DMSO) only.

FIGS. 12A-12D. Halogenated indoles bind specifically to the Nurr1 LBD, but not the RXRα LBD. Comparison of the MST binding isotherms for (FIG. 12A) 5-bromoindole, (FIG. 12B) 5-chloroindole, (FIG. 12C) 5,6-dibromoindole, and (FIG. 12D) 5,6-dichloroindole reveal saturable binding to the Nurr1 LBD (light grey circles), but not to the RXRα LBD (dark grey circles). Binding assays were carried out as described in Example 3. All experimental values are the result of three or more independent biological replicates standard deviation.

FIG. 13. Mutation of Arg563 within the Nurr1 LBD reduces the thermal stability of the protein. Melting curves were acquired using differential scanning fluorimetry (DSF). The Nurr1 LBD (4 μM), dissolved in 25 mM HEPES buffer, pH 7.4, 150 mM NaCl, 1×SYPRO™ Orange dye. The fluorescence response was normalized to the largest fluorescent value, defined as 100%, within each data set. The reported Tm (the inflection point of the sigmoidal curve) was calculated using the Boltzmann sigmoid equation: Y=bottom+(top-bottom)/(1+exp((Tm-x/slope)), where bottom and top are the values of the minimum and maximum intensities. Each data point is the average of at least three independent measurements±standard deviation; the curve for each variant is the result of the global fit to all replicates.

FIG. 14. Features of two distinct ligand binding sites within the Nurr1 LBD. The 566 site only accommodates 5-substituted indoles, requires His516 and Arg563 for binding, and upregulates the transcription of Th and Vmat2. The new site binds both 5- and 5,6-disubstituted indoles, but does not drive expression of either Th or Vmat2.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. In embodiments, the alkyl is fully saturated. In embodiments, the alkyl is monounsaturated. In embodiments, the alkyl is polyunsaturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. In embodiments, an alkenylene includes one or more double bonds. In embodiments, an alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —SCH₂CH₂, —S(O)CH₃, —CH₂—CH₂—S(O)₂—CH₃, —CH═CHO—CH₃, —Si(CH₃)₃, —CH₂—CH═N—OCH₃, —CH═CHN(CH₃)CH₃, —OCH₃, —OCH₂CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. In embodiments, a heteroalkenylene includes one or more double bonds. In embodiments, a heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

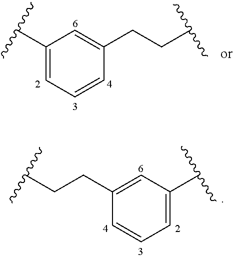 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R′ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1} As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{4.1}, R^{5.1} . . . R^{100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3}. R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R″ is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

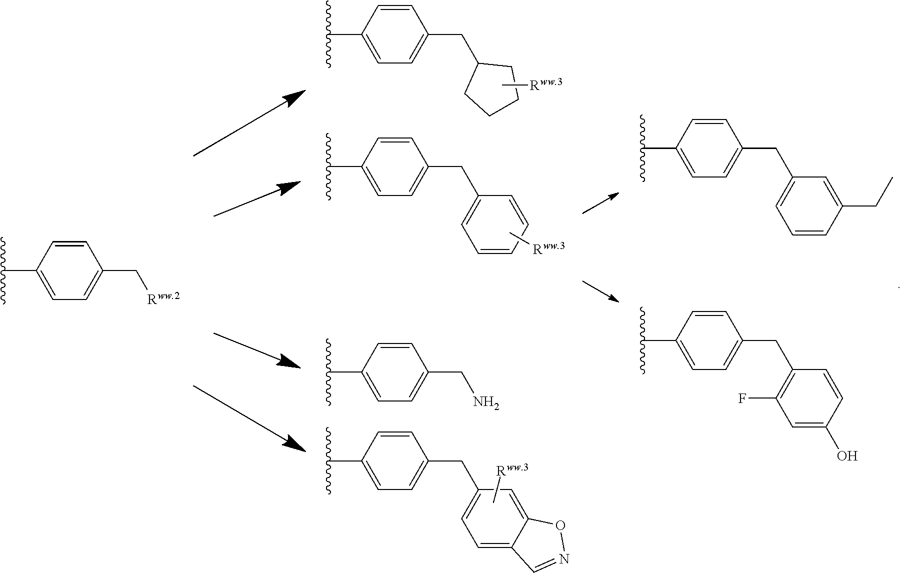

R^{WW.1} is independently oxo, halogen, —CX^{WW.1}₃, —CHX^{WW.1}₂, —CH₂X^{WW.1}, —OCX^{WW1}₃, —OCH₂X_{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g. substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.33}, —CHX^{LWW.32}, —CH₂X^{LWW.3}, —OCX^{LWW.33}, —OCH₂X^{LWW.3}, —OCHX^{LWW.32}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW}-1-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹₃ substituents are present, each R¹₃ substituent may be distinguished as R¹_{3A}, R¹_{3B}, R¹_{3C}, R¹_{3D}, etc., wherein each of R¹_{3A}, R¹_{3B}, R¹_{3C}, R¹_{3D}, etc. is defined within the scope of the definition of R¹₃ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “allosteric modulator” is used in accordance with its plain ordinary meaning and refers to a substance (e.g., compound) that binds to a receptor to change the receptor's response to stimulus. The site that an allosteric modulator binds to (i.e., an allosteric site) is not the same one to which an endogenous agonist of the receptor would bind (i.e., an orthosteric site). An allosteric modulator can alter (e.g., increase or decrease) the affinity and efficacy of other substances acting on a receptor. A “positive allosteric modulator” or “PAM” refers to an allosteric modulator that increases agonist affinity and/or efficacy. A “negative allosteric modulator” or “NAM” refers to an allosteric modulator that lowers agonist affinity and/or efficacy.

The term “allosteric site” is used in accordance with its plain ordinary meaning and refers to a binding site on an enzyme that is not the active site. In embodiments, binding of a substance (e.g., compound) to an allosteric site results in a conformational change of the enzyme. In embodiments, binding of a substance (e.g., compound) to an allosteric site results in modulation (e.g., activation or inhibition) of the enzyme's activity.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a neurodegenerative disease. In embodiments, the disease is a cancer.

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, scleroderma, and atopic dermatitis.

As used herein, the term “eye disease” refers to a disease or condition characterized by eye problems (e.g., an increased level of eye problems compared to a control such as a healthy person not suffering from a disease). Examples of eye diseases include, but are not limited to, cataract (e.g., congenital cataract), optic nerve disorders (e.g., glaucoma), retinal disorders, macular degeneration, diabetic eye problems, and conjunctivitis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

The compounds described herein can be co-administered with conventional neurodegenerative disease treatments including, but not limited to, Parkinson's disease treatments such as levodopa, carbidopa, selegiline, amantadine, donepezil, galanthamine, rivastigmine, tacrine, dopamine agonists (e.g., bromocriptine, pergolide, pramipexole, ropinirole), anticholinergic drugs (e.g., trihexyphenidyl, benztropine, biperiden, procyclidine), and catechol-O-methyl-transferase inhibitors (e.g., tolcapone, entacapone).

The compounds described herein can also be co-administered with conventional anti-inflammatory disease treatments including, but not limited to, analgesics (e.g., acetaminophen, duloxetine), nonsteroidal anti-inflammatory drugs (e.g., aspirin, ibuprofen, naproxen, diclofenac), corticosteroids (e.g., prednisone, betamethasone, cortisone, dexamethasone, hydrocortisone, methylprednisolone, prednisolone), and opoids (e.g., codeine, fentanyl, hydrocodone, hydromorphone, morphine, meperidine, oxycodone).

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safmgol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol.TM (i.e., paclitaxel), Taxotere.TM, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/IKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent.

The term “associated” or “associated with” in the context of a substance or substance activity or function associated with a disease (e.g., a protein associated disease, disease associated with a cellular component) means that the disease (e.g., neurodegenerative disease, cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). For example, a neurodegenerative disease associated with a protein aggregate may be a neurodegenerative disease that results (entirely or partially) from aberrant protein aggregation or a neurodegenerative disease wherein a particular symptom of the disease is caused (entirely or partially) by aberrant protein aggregation. As used herein, what is described as being associated with a disease, if a causative agent, could be a target for treatment of the disease. For example, a neurodegenerative disease associated with aberrant protein aggregation or a protein aggregate associated neurodegenerative disease, may be treated with a protein aggregate modulator.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond. In some embodiments, the electrophilic substituent of the compound is capable of reacting with a cysteine residue. In some embodiments, the electrophilic substituent is capable of forming a covalent bond with a cysteine residue and may be referred to as a “covalent cysteine modifier moiety” or “covalent cysteine modifier substituent.” The covalent bond formed between the electrophilic substituent and the sulfhydryl group of the cysteine may be a reversible or irreversible bond. In some embodiments, the electrophilic substituent of the compound is capable of reacting with a lysine residue. In some embodiments, the electrophilic substituent of the compound is capable of reacting with a serine residue. In some embodiments, the electrophilic substituent of the compound is capable of reacting with a methionine residue.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. Instead of a primary sequence alignment, a three dimensional structural alignment can also be used, e.g., where the structure of the selected protein is aligned for maximum correspondence with the human protein and the overall structures compared. In this case, an amino acid that occupies the same essential position as a specified amino acid in the structural model is said to correspond to the specified residue. For example, a selected residue in a selected protein corresponds to Arg563 of a Nurr1 protein (e.g., human Nurr1 protein or SEQ ID NO:1) when the selected residue occupies the same essential spatial or other structural relationship as Arg563 in a Nurr1 protein (e.g., a human Nurr1 protein or SEQ ID NO:1). In some embodiments, where a selected protein is aligned for maximum homology with the Nurr1 protein, the position in the aligned selected protein aligning with Arg563 is said to correspond to Arg563 of the Nurr1 protein (e.g., a human Nurr1 protein or SEQ ID NO:1). Instead of a primary sequence alignment, a three dimensional structural alignment can also be used, e.g., where the structure of the selected protein is aligned for maximum correspondence with the Nurr1 protein (e.g., a human Nurr1 protein or SEQ ID NO:1) and the overall structures compared. In this case, an amino acid that occupies the same essential position as Arg563 of a Nurr1 protein (e.g., a human Nurr1 protein or SEQ ID NO:1) in the structural model is said to correspond to the Arg563 residue. Another example is wherein a selected residue in a selected protein corresponds to Arg563 in a Nurr1 protein (e.g., a human Nurr1 protein or SEQ ID NO:1) when the selected residue (e.g., arginine residue) occupies essentially the same sequence, spatial, or other structural position within the protein as Arg563 in the Nurr1 protein (e.g., a human Nurr1 protein or SEQ ID NO:1).

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “Nurr1” or “NR4A2” refers to the protein that in humans is encoded by the NR4A2 gene. Nurr1 is a nuclear receptor and plays a key role in the maintenance of the dopaminergic system of the brain. The term “Nurr1” may refer to the nucleotide sequence or protein sequence of human NR4A.2 (e.g., Entrez 4929, Uniprot P43354, RefSeq NM_006186.3, or RefSeq NP_006177.1). In embodiments, Nurr1 ligand binding domain has the following amino acid sequence:

(SEQ ID NO: 1)
MPCVQAQYGSSPQGASPASQSYSYHSSGEYSSDFLTPEFVKFSMDLTNTE
ITATTSLPSFSTFMDNYSTGYDVKPPCLYQMPLSGQQSSIKVEDIQMHNY
QQHSHLPPQSEEMMPHSGSVYYKPSSPPTPTTPGFQVQHSPMWDDPGSLH
NFHQNYVATTHMIEQRKTPVSRLSLFSFKQSPPGTPVSSCQMRFDGPLHV
PMNPEPAGSHHVVDGQTFAVPNPIRKPASMGFPGLQIGHASQLLDTQVPS
PPSRGSPSNEGLCAVCGDNAACQHYGVRTCEGCKGFFKRTVQKNAKYVCL
ANKNCPVDKRRRNRCQYCRFQKCLAVGMVKEVVRTDSLKGRRGRLPSKPK
SPQEPSPPSPPVSLISALVRAHVDSNPAMTSLDYSRFQANPDYQMSGDDT
QHIQQFYDLLTGSMEIIRGWAEKIPGFADLPKADQDLLFESAFLELFVLR
LAYRSNPVEGKLIFCNGVVLHRLQCVRGFGEWIDSIVEFSSNLQNMNIDI
SAFSCIAALAMVTERHGLKEPKRVEELQNKIVNCLKDHVTFNNGGLNRPN
YLSKLLGKLPELRTLCTQGLQRIFYLKLEDLVPPPAIIDKLFLDTLPF.

The term “Tyrosine hydroxylase” or “Tyrosine 3-monooxygenase” refers to the enzyme responsible for catalyzing the conversion of the amino acid L-tyrosine to L-3,4-dihydroxyphenylalanine (L-DOPA). In humans, tyrosine hydroxylase is encoded by the TH gene. The term “TH” may refer to the nucleotide sequence or protein sequence of human TH (e.g., Entrez 7054, Uniprot P07101, RefSeq NM_000360.3, RefSeq NM_199292.2, RefSeq NM_199293.2, RefSeq NP_000351.2, RefSeq NP_954986.2, or RefSeq NP_954987.2). In embodiments, TH has the following amino acid sequence:

(SEQ ID NO: 2)
MPTPDATTPQAKGFRRAVSELDAKQAEAIMVRGQGAPGPSLTGSPWPGTA
APAASYTPTPRSPRFIGRRQSLIEDARKEREAAVAAAAAAVPSEPGDPLE
AVAFEEKEGKAVLNLLFSPRATKPSALSRAVKVFETFEAKIHHLETRPAQ
RPRAGGPHLEYFVRLEVRRGDLAALLSGVRQVSEDVRSPAGPKVPWFPRK
VSELDKCHHLVTKFDPDLDLDHPGFSDQVYRQRRKLIAEIAFQYRHGDPI
PRVEYTAEEIATWKEVYTTLKGLYATHACGEHLEAFALLERFSGYREDNI
PQLEDVSRFLKERTGFQLRPVAGLLSARDFLASLAFRVFQCTQYIRHASS
PMHSPEPDCCHELLGHVPMLADRTFAQFSQDIGLASLGASDEEIEKLSTL
YWFTVEFGLCKQNGEVKAYGAGLLSSYGELLHCLSEEPEIRAFDPEAAAV
QPYQDQTYQSVYFVSESFSDAKDKLRSYASRIQRPFSVKFDPYTLAIDVL
DSPQAVRRSLEGVQDELDTLAHALSAIG.

The term “Dopamine receptor D₂” or “D2R” refers to the dopamine receptor whose activity is mediated by G proteins which inhibit adenylyl cyclase. In humans, dopamine receptor D₂ is encoded by the DRD2 gene. The term “DRD2” may refer to the nucleotide sequence or protein sequence of human DRD2 (e.g., Entrez 1813, Uniprot P14416, RefSeq NM_016574.3, RefSeq NM_000795.3, RefSeq NP_000786.1, or RefSeq NP_057658.2). In embodiments, DRD2 has the following amino acid sequence:

(SEQ ID NO: 3)
MDPLNLSWYDDDLERQNWSRPFNGSDGKADRPHYNYYATLLTLLIAVIVF
GNVLVCMAVSREKALQTTTNYLIVSLAVADLLVATLVMPWVVYLEVVGEW
KFSRIHCDIFVTLDVMMCTASILNLCAISIDRYTAVAMPMLYNTRYSSKR
RVTVMISIVWVLSFTISCPLLFGLNNADQNECIIANPAFVVYSSIVSFYV
PFIVTLLVYIKIYIVLRRRRKRVNTKRSSRAFRAHLRAPLKGNCTHPEDM
KLCTVIMKSNGSFPVNRRRVEAARRAQELEMEMLSSTSPPERTRYSPIPP
SHHQLTLPDPSHHGLHSTPDSPAKPEKNGHAKDHPKIAKIFEIQTMPNGK
TRTSLKTMSRRKLSQQKEKKATQMLAIVLGVFIICWLPFFITHILNIHCD
CNIPPVLYSAFTWLGYVNSAVNPIIYTTFNIEFRKAFLKILHC.

The term “Vesicular monoamine transporter 2” or “VMAT2” refers to the integral membrane protein that transports neurotransmitters such as dopamine, norepinephrine, serotonin, and histamine, from cellular cytosol into synaptic vesicles. The term “VMAT2” may refer to the nucleotide sequence or protein sequence of human VMAT2 (e.g., Entrez 6571, Uniprot Q05940, RefSeq NM_003054.4, or RefSeq NP_003045.2). In embodiments, VMAT2 has the following amino acid sequence:

(SEQ ID NO: 4)
MALSELALVRWLQESRRSRKLILFIVFLALLLDNMLLTVVVPIIPSYLYS
IKHEKNATEIQTARPVHTASISDSFQSIFSYYDNSTMVTGNATRDLTLHQ
TATQHMVTNASAVPSDCPSEDKDLLNENVQVGLLFASKATVQLITNPFIG
LLTNRIGYPIPIFAGFCIMFVSTIMFAFSSSYAFLLIARSLQGIGSSCSS
VAGMGMLASVYTDDEERGNVMGIALGGLAMGVLVGPPFGSVLYEFVGKTA
PFLVLAALVLLDGAIQLFVLQPSRVQPESQKGTPLTTLLKDPYILIAAGS
ICFANMGIAMLEPALPIWMMETMCSRKWQLGVAFLPASISYLIGTNIFGI
LAHKMGRWLCALLGMIIVGVSILCIPFAKNIYGLIAPNFGVGFAIGMVDS
SMMPIMGYLVDLRHVSVYGSVYAIADVAFCMGYAIGPSAGGAIAKAIGFP
WLMTIIGIIDILFAPLCFFLRSPPAKEEKMAILMDHNCPIKTKMYTQNNI
QSYPIGEDEESESD.

The terms “dopa decarboxylase” and “DDC” refer to a protein (including homologs, isoforms, and functional fragments thereof) that catalyzes the decarboxylation of L-3,4-dihydroxyphenylalanine (DOPA) to dopamine. The term includes any recombinant or naturally-occurring form of DDC variants thereof that maintain DDC activity (e.g., within at least 30%, 40%, 50%, 60%, 70%, 80%, 90%, 95%, or 100% activity compared to wildtype DDC). In embodiments, the DDC protein encoded by the DDC gene has the amino acid sequence set forth in or corresponding to UniProt P20711, RefSeq (protein) NP 000781.1, RefSeq (protein) NP 001076440.1, RefSeq (protein) NP_001229815.1, RefSeq (protein) NP_001229816.1, RefSeq (protein) NP_001229817.1, RefSeq (protein) NP_001229818.1, or RefSeq (protein) NP 001229819.1. In embodiments, the DDC gene has the nucleic acid sequence set forth in RefSeq (mRNA) NM_000790.3, RefSeq (mRNA) NM_001082971.1, RefSeq (mRNA) NM 001242886.1, RefSeq (mRNA) NM_001242887.1, RefSeq (mRNA) NM_001242888.1, RefSeq (mRNA) NM 001242889.1, or RefSeq (mRNA) NM_001242890.1. In embodiments, the amino acid sequence or nucleic acid sequence is the sequence known at the time of filing of the present application.

The terms “dopamine transporter” and “DAT” refer to a protein (including homologs, isoforms, and functional fragments thereof) that transports dopamine out of the synaptic cleft back into cytosol. The term includes any recombinant or naturally-occurring form of DAT variants thereof that maintain DAT activity (e.g., within at least 30%, 40%, 50%, 60%, 70%, 80%, 90%, 95%, or 100% activity compared to wildtype DAT). In embodiments, the DAT protein encoded by the SLC6A.3 gene has the amino acid sequence set forth in or corresponding to Entrez 6531, UniProt Q01959, or RefSeq (protein) NP 001035.1. In embodiments, the SLC6A.3 gene has the nucleic acid sequence set forth in RefSeq (mRNA) NM_001044.4. In embodiments, the amino acid sequence or nucleic acid sequence is the sequence known at the time of filing of the present application.

The terms “brain-derived neurotrophic factor” and “BDNF” refer to a protein (including homologs, isoforms, and functional fragments thereof) of the neurotrophin family of growth factors. The term includes any recombinant or naturally-occurring form of BDNF variants thereof that maintain BDNF activity (e.g., within at least 30%, 40%, 50%, 60%, 70%, 80%, 90%, 95%, or 100% activity compared to wildtype BDNF). In embodiments, the BDNF protein encoded by the BDNF gene has the amino acid sequence set forth in or corresponding to Entrez 627, UniProt P23560, RefSeq (protein) NP_001137277.1, RefSeq (protein) NP_001137278.1, RefSeq (protein) NP_001137279.1, RefSeq (protein) NP_001137280.1, RefSeq (protein) NP_001137281.1, RefSeq (protein) NP_001137282.1, RefSeq (protein) NP_001137283.1, RefSeq (protein) NP_001137284.1, RefSeq (protein) NP_001137285.1, RefSeq (protein) NP_001137286.1, RefSeq (protein) NP_001137288.1. RefSeq (protein) NP_001700.2, RefSeq (protein) NP_733927.1, RefSeq (protein) NP_733928.1, RefSeq (protein) NP_733929.1. RefSeq (protein) NP_733930.1, or RefSeq (protein) NP 733931.1. In embodiments, the BDNF gene has the nucleic acid sequence set forth in RefSeq (mRNA) NM_001143805.1, RefSeq (mRNA) NM_001143806.1, RefSeq (mRNA) NM_001143807.1, RefSeq (mRNA) NM_00143808.1, RefSeq (mRNA) NM_001143809.1, RefSeq (mRNA) NM_001143810.1, RefSeq (mRNA) NM_001143811.1, RefSeq (mRNA) NM_001143812.1, RefSeq (mRNA) NM_001143813.1, RefSeq (mRNA) NM_001143814.1, RefSeq (mRNA) NM_001143816.1, RefSeq (mRNA) NM_001709.4, RefSeq (mRNA) NM 170731.4, RefSeq (mRNA) NM_170732.4, RefSeq (mRNA) NM_170733.3, RefSeq (mRNA) NM_170734.3, or RefSeq (mRNA) NM_170735.5. In embodiments, the amino acid sequence or nucleic acid sequence is the sequence known at the time of filing of the present application.

The terms “nerve growth factor” and “NGF” refer to a protein (including homologs, isoforms, and functional fragments thereof) involved in the regulation of growth, maintenance, proliferation, and survival of certain target neurons. The term includes any recombinant or naturally-occurring form of NGF variants thereof that maintain NGF activity (e.g., within at least 30%, 40%, 50%, 60%, 70%, 80%, 90%, 95%, or 100% activity compared to wildtype NGF). In embodiments, the NGF protein encoded by the NGF gene has the amino acid sequence set forth in or corresponding to Entrez 4803, UniProt P01138, or RefSeq (protein) NP 002497.2. In embodiments, the NGF gene has the nucleic acid sequence set forth in RefSeq (mRNA) NM_002506.2. In embodiments, the amino acid sequence or nucleic acid sequence is the sequence known at the time of filing of the present application.

The terms “glial cell line-derived neurotrophic factor” and “GDNF” refer to a protein (including homologs, isoforms, and functional fragments thereof) that promotes the survival of many types of neurons. The term includes any recombinant or naturally-occurring form of GDNF variants thereof that maintain GDNF activity (e.g., within at least 30%, 40%, 50%, 60%, 70%, 80%, 90%, 95%, or 100% activity compared to wildtype GDNF). In embodiments, the GDNF protein encoded by the GDNF gene has the amino acid sequence set forth in or corresponding to Entrez 2668, UniProt P39905, RefSeq (protein) NP_000505.1, RefSeq (protein) NP_001177397.1, RefSeq (protein) NP_001177398.1, RefSeq (protein) NP_001265027.1, or RefSeq (protein) NP 954701.1. In embodiments, the GDNF gene has the nucleic acid sequence set forth in RefSeq (mRNA) NM_000514.3, RefSeq (mRNA) NM_001190468.1, RefSeq (mRNA) NM_001190469.1, RefSeq (mRNA) NM_001278098.1, or RefSeq (mRNA) NM 199231.2. In embodiments, the amino acid sequence or nucleic acid sequence is the sequence known at the time of filing of the present application.

The terms “RET proto-oncogene” and “c-RET” refer to a protein (including homologs, isoforms, and functional fragments thereof) involved in cell proliferation, neuronal navigation, cell migration, and cell differentiation. The term includes any recombinant or naturally-occurring form of c-RET variants thereof that maintain c-RET activity (e.g., within at least 30%, 40%, 50%, 60%, 70%, 80%, 90%, 95%, or 100% activity compared to wildtype c-RET). In embodiments, the c-RET protein encoded by the RET gene has the amino acid sequence set forth in or corresponding to Entrez 5979, UniProt P07949, RefSeq (protein) NP_065681.1, or RefSeq (protein) NP_066124.1. In embodiments, the RET gene has the nucleic acid sequence set forth in RefSeq (mRNA) NM_020630.4 or RefSeq (mRNA) NM_020975.4. In embodiments, the amino acid sequence or nucleic acid sequence is the sequence known at the time of filing of the present application.

The terms “superoxide dismutase 1” and “SOD1” refer to a protein (including homologs, isoforms, and functional fragments thereof) involved in apoptosis. The term includes any recombinant or naturally-occurring form of SOD1 variants thereof that maintain SOD1 activity (e.g., within at least 30%, 40%, 50%, 60%, 70%, 80%, 90%, 95%, or 100% activity compared to wildtype SOD1). In embodiments, the SOD1 protein encoded by the SOD1 gene has the amino acid sequence set forth in or corresponding to Entrez 6647, UniProt P00441, or RefSeq (protein) NP_000445.1. In embodiments, the SOD1 gene has the nucleic acid sequence set forth in RefSeq (mRNA) NM_000454.4. In embodiments, the amino acid sequence or nucleic acid sequence is the sequence known at the time of filing of the present application.

The terms “tumor necrosis factor alpha” and “TNFα” refer to a protein (including homologs, isoforms, and functional fragments thereof) involved in cell signalling. The term includes any recombinant or naturally-occurring form of TNFα variants thereof that maintain TNFα activity (e.g., within at least 30%, 40%, 50%, 60%, 70%, 80%, 90%, 95%, or 100% activity compared to wildtype TNFα). In embodiments, the TNFα protein encoded by the TNF gene has the amino acid sequence set forth in or corresponding to Entrez 7124, UniProt P01375, or RefSeq (protein) NP_000585.2. In embodiments, the TNF gene has the nucleic acid sequence set forth in RefSeq (mRNA) NM_000594.3. In embodiments, the amino acid sequence or nucleic acid sequence is the sequence known at the time of filing of the present application.

The terms “inducible nitric oxide synthase” and “iNOS” refer to a protein (including homologs, isoforms, and functional fragments thereof) that produces nitric oxide. The term includes any recombinant or naturally-occurring form of iNOS variants thereof that maintain iNOS activity (e.g., within at least 30%, 40%, 50%, 60%, 70%, 80%, 90%, 95%, or 100% activity compared to wildtype iNOS). In embodiments, the iNOS protein encoded by the NOS2 gene has the amino acid sequence set forth in or corresponding to UniProt P35228 or RefSeq (protein) NP 000616.3. In embodiments, the NOS2 gene has the nucleic acid sequence set forth in RefSeq (mRNA) NM_000625.4. In embodiments, the amino acid sequence or nucleic acid sequence is the sequence known at the time of filing of the present application.

The terms “interleukin 1 beta” and “IL-1β” refer to a cytokine protein (including homologs, isoforms, and functional fragments thereof). The term includes any recombinant or naturally-occurring form of IL-1β variants thereof that maintain IL-1β activity (e.g., within at least 30%, 40%, 50%, 60%, 70%, 80%, 90%, 95%, or 100% activity compared to wildtype IL-1β). In embodiments, the IL-1β protein encoded by the IL1B gene has the amino acid sequence set forth in or corresponding to Entrez 3553, UniProt P01584, or RefSeq (protein) NP_000567.1. In embodiments, the IL1B gene has the nucleic acid sequence set forth in RefSeq (mRNA) NM 000576.2. In embodiments, the amino acid sequence or nucleic acid sequence is the sequence known at the time of filing of the present application.

The terms “Pituitary homeobox 3” and “PITX3” refer to a protein (including homologs, isoforms, and functional fragments thereof) of the RIEG/PITX homeobox family, which is in the bicoid class of homeodomain proteins and act as transcription factors. The term includes any recombinant or naturally-occurring form of PITX3 variants thereof that maintain PITX3 activity (e.g., within at least 30%, 40%, 50%, 60%, 70%, 80%, 90%, 95%, or 100% activity compared to wildtype PITX3). In embodiments, the PITX3 protein encoded by the Pitx3 gene has the amino acid sequence set forth in or corresponding to Entrez 5309, UniProt 075364, or RefSeq (protein) NP_005020.1. In embodiments, the Pitx3 gene has the nucleic acid sequence set forth in RefSeq (mRNA) NM_005029.3. In embodiments, the amino acid sequence or nucleic acid sequence is the sequence known at the time of filing of the present application.

The term “response element” is used in accordance with its plain ordinary meaning in the art and refers to a short sequence of DNA within a gene promoter or enhancer region that is able to bind specific transcription factors and regulate transcription of genes.

The term “NGFI-B response element” or “NBRE” refers to a response element for nerve growth factor IB (NGFI-B). In embodiments, the binding site has the nucleotide sequence 5′-AAAGGTCA.

The term “Nur-responsive element” or “NuRE” refers to a response element for homodimers or heterodimers of the NR4A family of nuclear receptors. In embodiments, NuRE has the nucleotide sequence 5′-TGATATTACCTCCAAATGCCA (SEQ ID NO:5).

The term “DR-5 response element” refers to a retinoic acid response element. In embodiments, the DR-5 response element has the nucleotide sequence 5′-GGTTCACCGAAAGGTCA (SEQ ID NO:6).

### II. Compounds

In an aspect is provided a compound having the formula

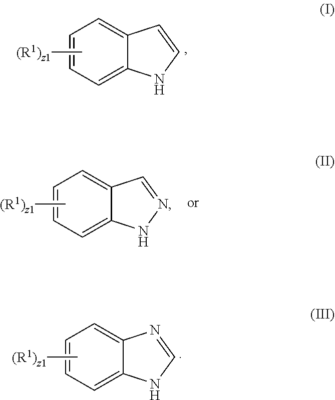

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SF₅, —SSR^{1D}, —SiR^{1A}R^{1B}R^{1C}, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

R^{1A}, R^{1B}, R^{1C}, and R^{1D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered); R^{1A} and R^{1B} substituents bonded to the same nitrogen atom may be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), or substituted (e.g., substituted with at least one substituent group, size-limited substituent group, or lower substituent group) or unsubstituted heteroaryl (e.g., 5 to 10 membered, 5 to 9 membered, or 5 to 6 membered).

The variable n1 is independently an integer from 0 to 4.

The variables m1 and v1 are independently 1 or 2.

X¹ is independently —F, —Cl, —Br, or —I.

The variable z1 is an integer from 0 to 6.

In embodiments, the compound has the formula

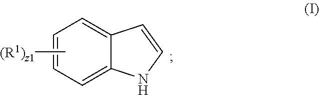

R¹ and z1 are as described herein, including in embodiments. In embodiments, the compound has the formula

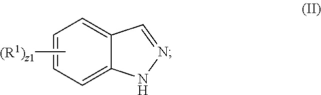

R¹ and z1 are as described herein, including in embodiments. In embodiments, the compound has the formula

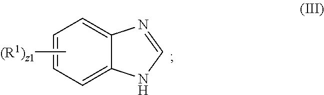

R¹ and z1 are as described herein, including in embodiments.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R¹ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —N H₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —CCl₃. In embodiments, R¹ is independently —CBr₃. In embodiments, R¹ is independently —CF₃. In embodiments, R¹ is independently —CI₃. In embodiments, R¹ is independently —CHCl₂. In embodiments, R¹ is independently —CHBr₂. In embodiments, R¹ is independently —CHF₂. In embodiments, R¹ is independently —CHI₂. In embodiments, R¹ is independently —CH₂Cl. In embodiments, R¹ is independently —CH_{2B}r. In embodiments, R¹ is independently —CH₂F. In embodiments, R¹ is independently —CH₂I. In embodiments, R¹ is independently —OCCl₃. In embodiments, R¹ is independently —OCF₃. In embodiments, R¹ is independently —OCBr₃. In embodiments, R¹ is independently —OCI₃. In embodiments, R¹ is independently —OCHCl₂. In embodiments, R¹ is independently —OCHBr₂. In embodiments, R¹ is independently —OCHI₂. In embodiments, R¹ is independently —OCHF₂. In embodiments, R¹ is independently —OCH₂Cl. In embodiments, R¹ is independently —OCH_{2B}r. In embodiments, R¹ is independently —OCH₂I. In embodiments, R¹ is independently —OCH₂F. In embodiments, R¹ is independently —CN. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently —SeH. In embodiments, R¹ is independently —SO₃H. In embodiments, R¹ is independently —OSO₃H. In embodiments, R¹ is independently —SO₂NH₂. In embodiments, R¹ is independently —NHNH₂. In embodiments, R¹ is independently —ONH₂. In embodiments, R¹ is independently —NHC(O)NHNH₂. In embodiments, R¹ is independently —NHC(O)NH₂. In embodiments, R¹ is independently —NHSO₂H. In embodiments, R¹ is independently —NHC(O)H. In embodiments, R¹ is independently —NHC(O)OH. In embodiments, R¹ is independently —NHOH. In embodiments, R¹ is independently —N₃. In embodiments, R¹ is independently —SF₅. In embodiments, R¹ is independently —SP(O)(OH)₂. In embodiments, R¹ is independently substituted or unsubstituted alkyl. In embodiments, R¹ is independently substituted or unsubstituted C₁-C₄ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted tert-butyl. In embodiments, R¹ is independently substituted or unsubstituted heteroalkyl. In embodiments, R¹ is independently substituted or unsubstituted 2 to 5 membered heteroalkyl. In embodiments, R¹ is independently unsubstituted methoxy. In embodiments, R¹ is independently unsubstituted ethoxy. In embodiments, R¹ is independently unsubstituted propoxy. In embodiments, R¹ is independently unsubstituted n-propoxy. In embodiments, R¹ is independently unsubstituted isopropoxy. In embodiments, R¹ is independently unsubstituted butoxy. In embodiments, R¹ is independently unsubstituted n-butoxy. In embodiments, R¹ is independently unsubstituted tert-butoxy. In embodiments, R¹ is independently substituted or unsubstituted cycloalkyl. In embodiments, R¹ is independently substituted or unsubstituted C₃-C₈ cycloalkyl. In embodiments, R¹ is independently substituted or unsubstituted heterocycloalkyl. In embodiments, R¹ is independently substituted or unsubstituted 3 to 8 membered heterocycloalkyl. In embodiments, R¹ is independently substituted or unsubstituted aryl. In embodiments, R¹ is independently substituted or unsubstituted C₆-C₁₀ aryl. In embodiments, R¹ is independently substituted or unsubstituted phenyl. In embodiments, R¹ is independently substituted or unsubstituted heteroaryl. In embodiments, R¹ is independently substituted or unsubstituted 5 to 10 membered heteroaryl.

In embodiments, z1 is 0. In embodiments, z1 is 1. In embodiments, z1 is 2. In embodiments, z1 is 3. In embodiments, z1 is 4. In embodiments, z1 is 5. In embodiments, z1 is 6.

In embodiments, the compound has the formula

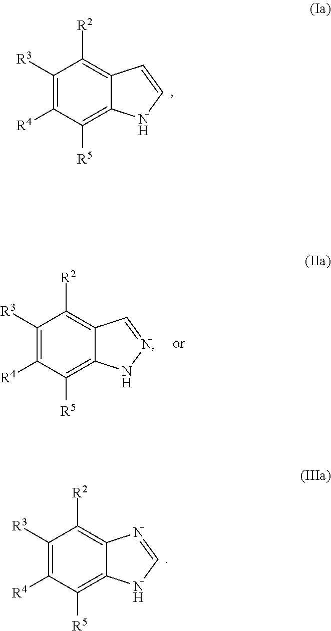

R² is hydrogen, halogen, —CX²₃, —CHX²₂, —CH₂X², —OCX²₃, —OCH₂X², —OCHX²₂, —CN, —SO₂R^{2D}, —SOᵥ₂NR^{2A}R^{2B}, —NHC(O)NR^{2A}R^{2B}, —N(O)ₘ₂, —N^{2A}R^{2B}, —C(O)R^{2C}, —SC(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, —SF₅, —SSR^{2D}, —SiR^{2A}R^{2B}R^{2C}, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —SC(O)R^{3C}, —C(O)OR^{3C}, —C(O)NR^{3A}R^{3B}, —OR^{3D}, —SR^{3D}, —SeR^{3D}, —NR^{3A}SO₂R^{3D}, —NR^{3A}C(O)R^{3C}, —NR^{3A}C(O)OR^{3C}, —NR^{3A}OR^{3C}, —N₃, —SF₅, —SSR^{3D}, —SiR^{3A}R^{3B}R^{3C}, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —SC(O)R^{4C}, —C(O)OR^{4C}, —C(O)NR^{4A}R^{4B}, —OR^{4D}, —SR^{4D}, —SeR^{4D}, —N_{4A}SO₂R^{4D}, —NR^{4A}C(O)R^{4C}, —NR^{4A}C(O)OR^{4C}, —NR^{4A}OR^{4C}, —N₃, —SF₅, —SSR^{4D}, —SiR^{4A}R^{4B}R^{4C}, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —SC(O)R^{5C}, —C(O)OR^{5C}, —C(O)NR^{5A}R^{5B}, —OR^{5D}, —SR^{5D}, —SeR^{5D}, —NR^{5A}SO₂R^{5D}, —NR^{5A}C(O)R^{5C}, —NR^{5A}C(O)OR^{5C}, —NR^{5A}OR^{5C}, —N₃, —SF₅, —SSR^{5D}, —SiR^{5A}R^{5B}R^{5C}, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered); R^{2A} and R^{2B} substituents bonded to the same nitrogen atom may be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), or substituted (e.g., substituted with at least one substituent group, size-limited substituent group, or lower substituent group) or unsubstituted heteroaryl (e.g., 5 to 10 membered, 5 to 9 membered, or 5 to 6 membered); R^{3A} and R^{3B} substituents bonded to the same nitrogen atom may be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), or substituted (e.g., substituted with at least one substituent group, size-limited substituent group, or lower substituent group) or unsubstituted heteroaryl (e.g., 5 to 10 membered, 5 to 9 membered, or 5 to 6 membered); R^{4A} and R^{4B} substituents bonded to the same nitrogen atom may be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), or substituted (e.g., substituted with at least one substituent group, size-limited substituent group, or lower substituent group) or unsubstituted heteroaryl (e.g., 5 to 10 membered, 5 to 9 membered, or 5 to 6 membered); R^{5A} and R^{5B} substituents bonded to the same nitrogen atom may be joined to form a substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), or substituted (e.g., substituted with at least one substituent group, size-limited substituent group, or lower substituent group) or unsubstituted heteroaryl (e.g., 5 to 10 membered, 5 to 9 membered, or 5 to 6 membered).

The variables n2, n3, n4, and n5 are independently an integer from 0 to 4.

The variables m2, m3, m4, m5, v2, v3, v4, and v5 are independently 1 or 2.

X², X³, X⁴, and X⁵ are independently —F, —Cl, —Br, or —I.

In embodiments, the compound has the formula

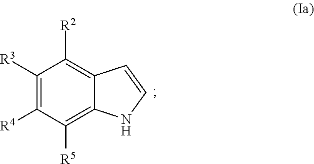

R², R³, R⁴, and R⁵ are as described herein, including in embodiments. In embodiments, the compound has the formula

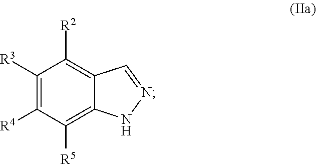

R², R³, R⁴, and R⁵ are as described herein, including in embodiments. In embodiments, the compound has the formula

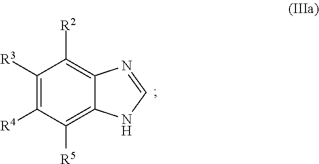

R², R³, R⁴, and R⁵ are as described herein, including in embodiments.

In embodiments, the compound has the formula

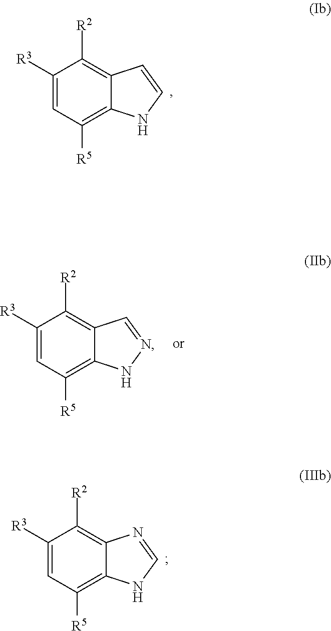

R², R³, and R⁵ are as described herein, including in embodiments. In embodiments, the compound has the formula

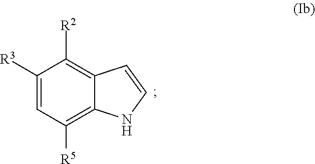

R², R³, and R⁵ are as described herein, including in embodiments. In embodiments, the compound has the formula

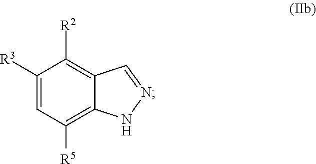

R², R³, and R⁵ are as described herein, including in embodiments. In embodiments, the compound has the formula

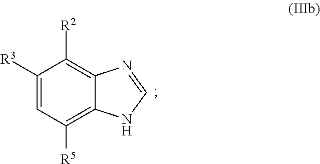

R², R³, and R⁵ are as described herein, including in embodiments.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

In embodiments, R² is hydrogen, halogen, —CF₃, —CH₂F, —CHF₂, —CN, —OH, —NH₂, —COOH, —CONH₂, —OCF₃, —OCHF₂, —OCH₂F, substituted or unsubstituted C₁-C₄ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

In embodiments, R² is hydrogen. In embodiments, R² is halogen. In embodiments, R² is —F. In embodiments, R² is —Cl. In embodiments, R² is —Br. In embodiments, R² is —I. In embodiments, R² is —CCl₃. In embodiments, R² is —CBr₃. In embodiments, R² is —CF₃. In embodiments, R² is —CI₃. In embodiments, R² is —CHCl₂. In embodiments, R² is —CHBr₂. In embodiments, R² is —CHF₂. In embodiments, R² is —CHI₂. In embodiments, R² is —CH₂Cl. In embodiments, R² is —CH_{2B}r. In embodiments, R² is —CH₂F. In embodiments, R² is —CH₂I. In embodiments, R² is —OCCl₃. In embodiments, R² is —OCF₃. In embodiments, R² is —OCBr₃. In embodiments, R² is —OCI₃. In embodiments, R² is —OCHCl₂. In embodiments, R² is —OCHBr₂. In embodiments, R² is —OCHI₂. In embodiments, R² is —OCHF₂. In embodiments, R² is —OCH₂Cl. In embodiments, R² is —OCH_{2B}r. In embodiments, R² is —OCH₂I. In embodiments, R² is —OCH₂F. In embodiments, R² is —CN. In embodiments, R² is —OH. In embodiments, R² is —NH₂. In embodiments, R² is —COOH. In embodiments, R² is —CONH₂. In embodiments, R² is —NO₂. In embodiments, R² is —SH. In embodiments, R² is —SeH. In embodiments, R² is —SO₃H. In embodiments, R² is —OSO₃H. In embodiments, R² is —SO₂NH₂. In embodiments, R² is —NHNH₂. In embodiments, R² is —ONH₂. In embodiments, R² is —NHC(O)NHNH₂. In embodiments, R² is —NHC(O)NH₂. In embodiments, R² is —NHSO₂H. In embodiments, R² is —NHC(O)H. In embodiments, R² is —NHC(O)OH. In embodiments, R² is —NHOH. In embodiments, R² is —N₃. In embodiments, R² is —SF₅. In embodiments, R² is —SP(O)(OH)₂. In embodiments, R² is substituted or unsubstituted alkyl. In embodiments, R² is substituted or unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted tert-butyl. In embodiments, R² is substituted or unsubstituted heteroalkyl. In embodiments, R² is substituted or unsubstituted 2 to 5 membered heteroalkyl. In embodiments, R² is unsubstituted methoxy. In embodiments, R² is unsubstituted ethoxy. In embodiments, R² is unsubstituted propoxy. In embodiments, R² is unsubstituted n-propoxy. In embodiments, R² is unsubstituted isopropoxy. In embodiments, R² is unsubstituted butoxy. In embodiments, R² is unsubstituted n-butoxy. In embodiments, R² is unsubstituted tert-butoxy. In embodiments, R² is substituted or unsubstituted cycloalkyl. In embodiments, R² is substituted or unsubstituted C₃-C₈ cycloalkyl. In embodiments, R² is substituted or unsubstituted heterocycloalkyl. In embodiments, R² is substituted or unsubstituted 3 to 8 membered heterocycloalkyl. In embodiments, R² is substituted or unsubstituted aryl. In embodiments, R² is substituted or unsubstituted C₆-C₁₀ aryl. In embodiments, R² is substituted or unsubstituted phenyl. In embodiments, R² is substituted or unsubstituted heteroaryl. In embodiments, R² is substituted or unsubstituted 5 to 10 membered heteroaryl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A} and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R³ is hydrogen. In embodiments, R³ is halogen. In embodiments, R³ is —F. In embodiments, R³ is —Cl. In embodiments, R³ is —Br. In embodiments, R³ is —I. In embodiments, R³ is —Br or —Cl. In embodiments, R³ is —CCl₃. In embodiments, R³ is —CBr₃. In embodiments, R³ is —CF₃. In embodiments, R³ is —CI₃. In embodiments, R³ is —CHCl₂. In embodiments, R³ is —CHBr₂. In embodiments, R³ is —CHF₂. In embodiments, R³ is —CHI₂. In embodiments, R³ is —CH₂Cl. In embodiments, R³ is —CH_{2B}r. In embodiments, R³ is —CH₂F. In embodiments, R³ is —CH₂I. In embodiments, R³ is —OCCl₃. In embodiments, R³ is —OCF₃. In embodiments, R³ is —OCBr₃. In embodiments, R³ is —OCI₃. In embodiments, R³ is —OCHCl₂. In embodiments, R³ is —OCHBr₂. In embodiments, R³ is —OCHI₂. In embodiments, R³ is —OCHF₂. In embodiments, R³ is —OCH₂Cl. In embodiments, R³ is —OCH_{2B}r. In embodiments, R³ is —OCH₂I. In embodiments, R³ is —OCH₂F. In embodiments, R³ is —CN. In embodiments, R³ is —OH. In embodiments, R³ is —NH₂. In embodiments, R³ is —COOH. In embodiments, R³ is —CONH₂. In embodiments, R³ is —NO₂. In embodiments, R³ is —SH. In embodiments, R³ is —SeH. In embodiments, R³ is —SO₃H. In embodiments, R³ is —OSO₃H. In embodiments, R³ is —SO₂NH₂. In embodiments, R³ is —NHNH₂. In embodiments, R³ is —ONH₂. In embodiments, R³ is —NHC(O)NHNH₂. In embodiments, R³ is —NHC(O)NH₂. In embodiments, R³ is —NHSO₂H. In embodiments, R³ is —NHC(O)H. In embodiments, R³ is —NHC(O)OH. In embodiments, R³ is —NHOH. In embodiments, R³ is —N₃. In embodiments, R³ is —SF₅. In embodiments, R³ is —SP(O)(OH)₂. In embodiments, R³ is substituted or unsubstituted alkyl. In embodiments, R³ is substituted or unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted tert-butyl. In embodiments, R³ is substituted or unsubstituted heteroalkyl. In embodiments, R³ is substituted or unsubstituted 2 to 5 membered heteroalkyl. In embodiments, R³ is unsubstituted methoxy. In embodiments, R³ is unsubstituted ethoxy. In embodiments, R³ is unsubstituted propoxy. In embodiments, R³ is unsubstituted n-propoxy. In embodiments, R³ is unsubstituted isopropoxy. In embodiments, R³ is unsubstituted butoxy. In embodiments, R³ is unsubstituted n-butoxy. In embodiments, R³ is unsubstituted tert-butoxy. In embodiments, R³ is substituted or unsubstituted cycloalkyl. In embodiments, R³ is substituted or unsubstituted C₃-C₈ cycloalkyl. In embodiments, R³ is substituted or unsubstituted heterocycloalkyl. In embodiments, R³ is substituted or unsubstituted 3 to 8 membered heterocycloalkyl. In embodiments, R³ is substituted or unsubstituted aryl. In embodiments, R³ is substituted or unsubstituted C₆-C₁₀ aryl. In embodiments, R³ is substituted or unsubstituted phenyl. In embodiments, R³ is substituted or unsubstituted heteroaryl. In embodiments, R³ is substituted or unsubstituted 5 to 10 membered heteroaryl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R⁴ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, R⁴ is hydrogen. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CCl₃. In embodiments, R⁴ is —CBr₃. In embodiments, R⁴ is —CF₃. In embodiments, R⁴ is —CI₃. In embodiments, R⁴ is —CHCl₂. In embodiments, R⁴ is —CHBr₂. In embodiments, R⁴ is —CHF₂. In embodiments, R⁴ is —CHI₂. In embodiments, R⁴ is —CH₂Cl. In embodiments, R⁴ is —CH_{2B}r. In embodiments, R⁴ is —CH₂F. In embodiments, R⁴ is —CH₂I. In embodiments, R⁴ is —OCCl₃. In embodiments, R⁴ is —OCF₃. In embodiments, R⁴ is —OCBr₃. In embodiments, R⁴ is —OCI₃. In embodiments, R⁴ is —OCHCl₂. In embodiments, R⁴ is —OCHBr₂. In embodiments, R⁴ is —OCHI₂. In embodiments, R⁴ is —OCHF₂. In embodiments, R⁴ is —OCH₂Cl. In embodiments, R⁴ is —OCH_{2B}r. In embodiments, R⁴ is —OCH₂I. In embodiments, R⁴ is —OCH₂F. In embodiments, R⁴ is —CN. In embodiments, R⁴ is —OH. In embodiments, R⁴ is —NH₂. In embodiments, R⁴ is —COOH. In embodiments, R⁴ is —CONH₂. In embodiments, R⁴ is —NO₂. In embodiments, R⁴ is —SH. In embodiments, R⁴ is —SeH. In embodiments, R⁴ is —SO₃H. In embodiments, R⁴ is —OSO₃H. In embodiments, R⁴ is —SO₂NH₂. In embodiments, R⁴ is —NHNH₂. In embodiments, R⁴ is —ONH₂. In embodiments, R⁴ is —NHC(O)NHNH₂. In embodiments, R⁴ is —NHC(O)NH₂. In embodiments, R⁴ is —NHSO₂H. In embodiments, R⁴ is —NHC(O)H. In embodiments, R⁴ is —NHC(O)OH. In embodiments, R⁴ is —NHOH. In embodiments, R⁴ is —N₃. In embodiments, R⁴ is —SF₅. In embodiments, R⁴ is —SP(O)(OH)₂. In embodiments, R⁴ is substituted or unsubstituted alkyl. In embodiments, R⁴ is substituted or unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted tert-butyl. In embodiments, R⁴ is substituted or unsubstituted heteroalkyl. In embodiments, R⁴ is substituted or unsubstituted 2 to 5 membered heteroalkyl. In embodiments, R⁴ is unsubstituted methoxy. In embodiments, R⁴ is unsubstituted ethoxy. In embodiments, R⁴ is unsubstituted propoxy. In embodiments, R⁴ is unsubstituted n-propoxy. In embodiments, R⁴ is unsubstituted isopropoxy. In embodiments, R⁴ is unsubstituted butoxy. In embodiments, R⁴ is unsubstituted n-butoxy. In embodiments, R⁴ is unsubstituted tert-butoxy. In embodiments, R⁴ is substituted or unsubstituted cycloalkyl. In embodiments, R⁴ is substituted or unsubstituted C₃-C₈ cycloalkyl. In embodiments, R⁴ is substituted or unsubstituted heterocycloalkyl. In embodiments, R⁴ is substituted or unsubstituted 3 to 8 membered heterocycloalkyl. In embodiments, R⁴ is substituted or unsubstituted aryl. In embodiments, R⁴ is substituted or unsubstituted C₆-C₁₀ aryl. In embodiments, R⁴ is substituted or unsubstituted phenyl. In embodiments, R⁴ is substituted or unsubstituted heteroaryl. In embodiments, R⁴ is substituted or unsubstituted 5 to 10 membered heteroaryl.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5A} and R^{5B} substituents bonded to the same nitrogen atom are joined is substituted, it is substituted with at least one substituent group. In embodiments, when the substituted ring formed when R^{5A} and R^{5B} substituents bonded to the same nitrogen atom are joined is substituted, it is substituted with at least one size-limited substituent group. In embodiments, when the substituted ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

In embodiments, R⁵ is hydrogen, halogen, —CF₃, —CH₂F, —CHF₂, —CN, —OH, —NH₂, —COOH, —CONH₂, —OCF₃, —OCHF₂, —OCH₂F, substituted or unsubstituted C₁-C₄ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

In embodiments, R⁵ is hydrogen. In embodiments, R⁵ is halogen. In embodiments, R⁵ is —F. In embodiments, R⁵ is —Cl. In embodiments, R⁵ is —Br. In embodiments, R⁵ is —I. In embodiments, R⁵ is —CCl₃. In embodiments, R⁵ is —CBr₃. In embodiments, R⁵ is —CF₃. In embodiments, R⁵ is —CI₃. In embodiments, R⁵ is —CHCl₂. In embodiments, R⁵ is —CHBr₂. In embodiments, R⁵ is —CHF₂. In embodiments, R⁵ is —CHI₂. In embodiments, R⁵ is —CH₂Cl. In embodiments, R⁵ is —CH_{2B}r. In embodiments, R⁵ is —CH₂F. In embodiments, R⁵ is —CH₂I. In embodiments, R⁵ is —OCCl₃. In embodiments, R⁵ is —OCF₃. In embodiments, R⁵ is —OCBr₃. In embodiments, R⁵ is —OCI₃. In embodiments, R⁵ is —OCHCl₂. In embodiments, R⁵ is —OCHBr₂. In embodiments, R⁵ is —OCHI₂. In embodiments, R⁵ is —OCHF₂. In embodiments, R⁵ is —OCH₂Cl. In embodiments, R⁵ is —OCH_{2B}r. In embodiments, R⁵ is —OCH₂I. In embodiments, R⁵ is —OCH₂F. In embodiments, R⁵ is —CN. In embodiments, R⁵ is —OH. In embodiments, R⁵ is —NH₂. In embodiments, R⁵ is —COOH. In embodiments, R⁵ is —CONH₂. In embodiments, R⁵ is —NO₂. In embodiments, R⁵ is —SH. In embodiments, R⁵ is —SeH. In embodiments, R⁵ is —SO₃H. In embodiments, R⁵ is —OSO₃H. In embodiments, R⁵ is —SO₂NH₂. In embodiments, R⁵ is —NHNH₂. In embodiments, R⁵ is —ONH₂. In embodiments, R⁵ is —NHC(O)NHNH₂. In embodiments, R⁵ is —NHC(O)NH₂. In embodiments, R⁵ is —NHSO₂H. In embodiments, R⁵ is —NHC(O)H. In embodiments, R⁵ is —NHC(O)OH. In embodiments, R⁵ is —NHOH. In embodiments, R⁵ is —N₃. In embodiments, R⁵ is —SF₅. In embodiments, R⁵ is —SP(O)(OH)₂. In embodiments, R⁵ is substituted or unsubstituted alkyl. In embodiments, R⁵ is substituted or unsubstituted C₁-C₄ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted tert-butyl. In embodiments, R⁵ is substituted or unsubstituted heteroalkyl. In embodiments, R⁵ is substituted or unsubstituted 2 to 5 membered heteroalkyl. In embodiments, R⁵ is unsubstituted methoxy. In embodiments, R⁵ is unsubstituted ethoxy. In embodiments, R⁵ is unsubstituted propoxy. In embodiments, R⁵ is unsubstituted n-propoxy. In embodiments, R⁵ is unsubstituted isopropoxy. In embodiments, R⁵ is unsubstituted butoxy. In embodiments, R⁵ is unsubstituted n-butoxy. In embodiments, R⁵ is unsubstituted tert-butoxy. In embodiments, R⁵ is substituted or unsubstituted cycloalkyl. In embodiments, R⁵ is substituted or unsubstituted C₃-C₈ cycloalkyl. In embodiments, R⁵ is substituted or unsubstituted heterocycloalkyl. In embodiments, R⁵ is substituted or unsubstituted 3 to 8 membered heterocycloalkyl. In embodiments, R⁵ is substituted or unsubstituted aryl. In embodiments, R⁵ is substituted or unsubstituted C₆-C₁₀ aryl. In embodiments, R⁵ is substituted or unsubstituted phenyl. In embodiments, R⁵ is substituted or unsubstituted heteroaryl. In embodiments, R⁵ is substituted or unsubstituted 5 to 10 membered heteroaryl.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D.1}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B.1}, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5D}, R^{5D.1}, R^{5D.2}, and R^{5D.3}, respectively.

In embodiments, the compound is

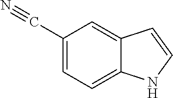 [SMILES: N#Cc1ccc2[nH]ccc2c1]

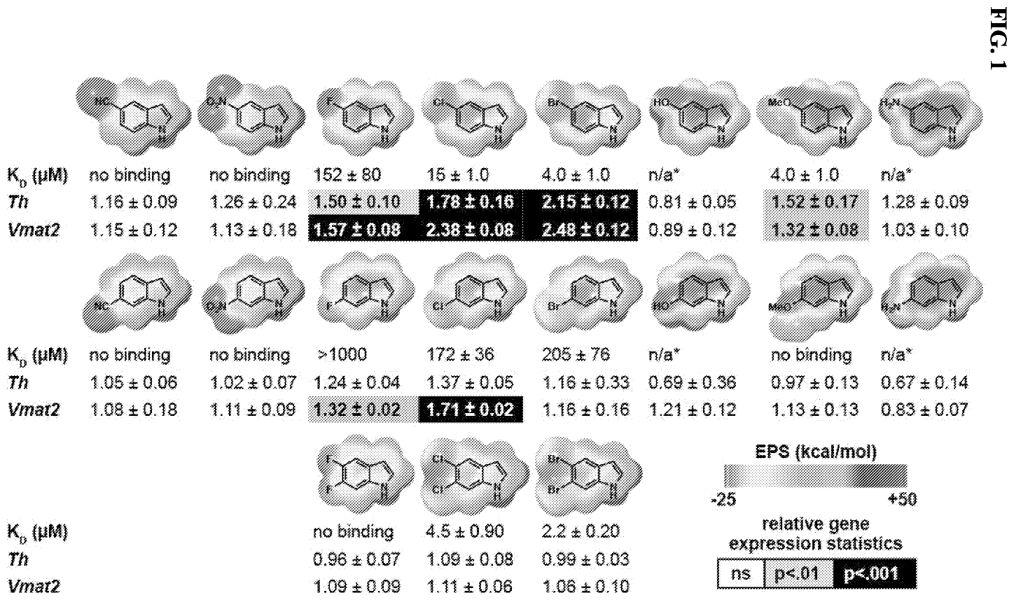

(FIG. 1, first row, first compound). In embodiments, the compound is

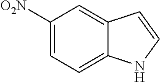 [SMILES: O=[N+]([O-])c1ccc2[nH]ccc2c1]

(FIG. 1, first row, second compound). In embodiments, the compound is

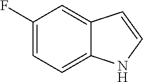

(FIG. 1, first row, third compound). In embodiments, the compound is

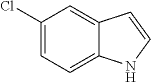

(FIG. 1, first row, fourth compound). In embodiments, the compound is

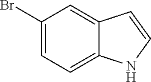

(FIG. 1, first row, fifth compound). In embodiments, the compound is

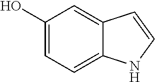

(FIG. 1, first row, sixth compound). In embodiments, the compound is

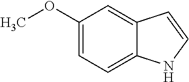

(FIG. 1, first row, seventh compound). In embodiments, the compound is

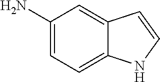

(FIG. 1, first row, eighth compound). In embodiments, the compound is

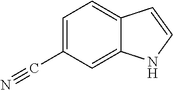 [SMILES: N#Cc1ccc2cc[nH]c2c1]

(FIG. 1, second row, first compound). In embodiments, the compound is

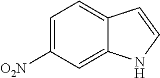 [SMILES: O=[N+]([O-])c1ccc2cc[nH]c2c1]

(FIG. 1, second row, second compound). In embodiments, the compound is

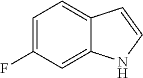

(FIG. 1, second row, third compound). In embodiments, the compound is

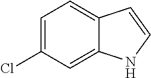

(FIG. 1, second row, fourth compound). In embodiments, the compound is

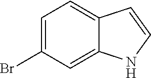

(FIG. 1, second row, fifth compound). In embodiments, the compound is

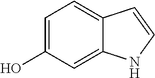

(FIG. 1, second row, sixth compound). In embodiments, the compound is

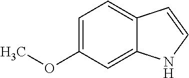

(FIG. 1, second row, seventh compound). In embodiments, the compound is

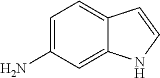

(FIG. 1, second row, eighth compound). In embodiments, the compound is

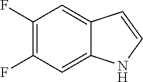 [SMILES: Fc1cc2cc[nH]c2cc1F]

(FIG. 1, third row, first compound). In embodiments, the compound is

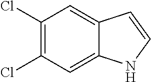 [SMILES: Clc1cc2cc[nH]c2cc1Cl]

(FIG. 1, third row, second compound). In embodiments, the compound is

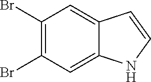 [SMILES: Brc1cc2cc[nH]c2cc1Br]

(FIG. 1, third row, third compound). In embodiments, the compound is

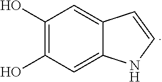 [SMILES: Oc1cc2cc[nH]c2cc1O]

In embodiments, the compound is not

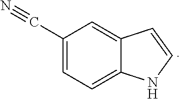 [SMILES: N#Cc1ccc2[nH]ccc2c1]

In embodiments, the compound is not

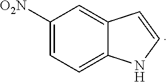 [SMILES: O=[N+]([O-])c1ccc2[nH]ccc2c1]

In embodiments, the compound is not

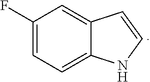

In embodiments, the compound is not

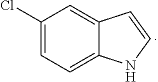

In embodiments, the compound is not

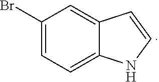

In embodiments, the compound is not

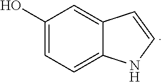

In embodiments, the compound is not

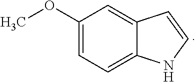

In embodiments, the compound is not

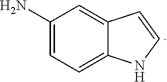

In embodiments, the compound is not

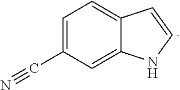 [SMILES: N#Cc1ccc2cc[nH]c2c1]

In embodiments, the compound is not

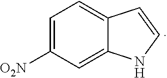 [SMILES: O=[N+]([O-])c1ccc2cc[nH]c2c1]

In embodiments, the compound is not

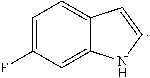

In embodiments, the compound is not

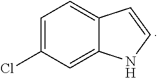

In embodiments, the compound is not

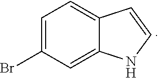

In embodiments, the compound is not

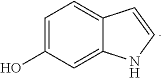

In embodiments, the compound is not

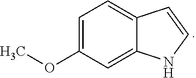

In embodiments, the compound is not

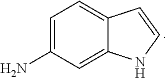

In embodiments, the compound is not

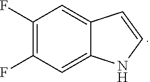 [SMILES: Fc1cc2cc[nH]c2cc1F]

H In embodiments, the compound is not

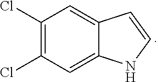 [SMILES: Clc1cc2cc[nH]c2cc1Cl]

In embodiments, the compound is not

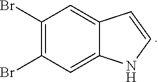 [SMILES: Brc1cc2cc[nH]c2cc1Br]

In embodiments, the compound is not

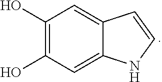 [SMILES: Oc1cc2cc[nH]c2cc1O]

In embodiments, the compound binds (e.g., noncovalently) Nurr1 (e.g., human Nurr1). In embodiments, the compound binds (e.g., noncovalently) the Nurr1 (e.g., human Nurr1) active site. In embodiments, the compound binds (e.g., noncovalently) a Nurr1 (e.g., human Nurr1) allosteric site.

In embodiments, the compound (e.g., described herein) is a positive allosteric modulator. In embodiments, the compound (e.g., described herein) is a negative allosteric modulator.

In embodiments, the compound contacts an amino acid corresponding to Arg515 of human Nurr1 (e.g., SEQ ID NO:1). In embodiments, the compound contacts an amino acid corresponding to Arg563 of human Nurr1 (e.g., SEQ ID NO:1). In embodiments, the compound contacts an amino acid corresponding to Glu445 of human Nurr1 (e.g., SEQ ID NO:1). In embodiments, the compound contacts an amino acid corresponding to His516 of human Nurr1 (e.g., SEQ ID NO:1).

In embodiments, the compound stabilizes a Nurr1 monomer. In embodiments, the compound stabilizes a Nurr1 homodimer. In embodiments, the compound stabilizes a head-to-tail Nurr1 homodimer. In embodiments, the compound stabilizes a Nurr1 heterodimer. In embodiments, the Nurr1 heterodimer is a heterodimer with RXRα.

In embodiments, the compound contacts a Nurr1 monomer. In embodiments, the compound contacts a Nurr1 homodimer. In embodiments, the compound contacts a head-to-tail Nurr1 homodimer. In embodiments, the compound contacts a Nurr1 heterodimer. In embodiments, the Nurr1 heterodimer is a heterodimer with RXRα.

In embodiments, the compound binds a Nurr1 monomer. In embodiments, the compound binds a Nurr1 homodimer. In embodiments, the compound binds a head-to-tail Nurr1 homodimer. In embodiments, the compound binds a Nurr1 heterodimer. In embodiments, the Nurr1 heterodimer is a heterodimer with RXRα.

In embodiments, the compound precludes the formation of Nurr1:RXR heterodimers. In embodiments, the compound inhibits the formation of Nurr1:RXR heterodimers. In embodiments, compound binding to Nurr1 inhibits the resulting compound:Nurr1 complex from binding to RXR.

In embodiments, the compound binds Nurr1 and induces Nurr1 binding to a NBRE, a NuRE, or a DR-5 response element. In embodiments, the compound binds Nurr1 and induces Nurr1 binding to a NBRE. In embodiments, the compound binds Nurr1 and induces Nurr1 binding to a NuRE. In embodiments, the compound binds Nurr1 and induces Nurr1 binding to a DR-5 response element.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the pharmaceutical composition includes an effective amount of a second agent, wherein the second agent is an agent for treating a neurodegenerative disease. In embodiments, the neurodegenerative disease is Parkinson's disease. In embodiments, the second agent is a Parkinson's disease drug, for example, levodopa, carbidopa, selegiline, amantadine, donepezil, galanthamine, rivastigmine, tacrine, bromocriptine, pergolide, pramipexole, ropinirole, trihexyphenidyl, benztropine, biperiden, procyclidine, tolcapone, or entacapone. In embodiments, the pharmaceutical composition includes a therapeutically effective amount of the second agent.

In embodiments, the pharmaceutical composition includes an effective amount of a second agent, wherein the second agent is an agent for treating an inflammatory disease, for example, acetaminophen, duloxetine, aspirin, ibuprofen, naproxen, diclofenac, prednisone, betamethasone, cortisone, dexamethasone, hydrocortisone, methylprednisolone, prednisolone, codeine, fentanyl, hydrocodone, hydromorphone, morphine, meperidine, or oxycodone. In embodiments, the pharmaceutical composition includes a therapeutically effective amount of the second agent.

In embodiments, the pharmaceutical composition includes an effective amount of a second agent, wherein the second agent is an anti-cancer agent.

### IV. Methods of Use

In an aspect is provided a method of treating a disease associated with dysregulation and/or degeneration of dopaminergic neurons in the central nervous system of a subject in need thereof, the method including administering to the subject in need thereof a therapeutically effective amount of a compound described herein, or a pharmaceutically acceptable salt thereof. In embodiments, the method does not include administering a compound for treating (e.g., effective in treating) a disease associated with dysregulation and/or degeneration of dopaminergic neurons in the central nervous system other than a compound described herein. In embodiments, the compound for treating (e.g., effective in treating) a disease associated with dysregulation and/or degeneration of dopaminergic neurons in the central nervous system is a compound for treating (e.g., effective in treating) Parkinson's disease, Alzheimer's disease, multiple sclerosis, amyotrophic lateral sclerosis, schizophrenia, or drug addiction. In embodiments the compound for treating (e.g., effective in treating) a disease associated with dysregulation and/or degeneration of dopaminergic neurons in the central nervous system is a compound for treating (e.g., effective in treating) cancer (e.g., an anti-cancer compound).

In embodiments, the disease associated with dysregulation and/or degeneration of dopaminergic neurons is Parkinson's disease, Alzheimer's disease, multiple sclerosis, amyotrophic lateral sclerosis, schizophrenia, or drug addiction. In embodiments, the disease associated with dysregulation and/or degeneration of dopaminergic neurons is Parkinson's disease. In embodiments, the disease is Alzheimer's disease. In embodiments, the disease associated with dysregulation and/or degeneration of dopaminergic neurons is multiple sclerosis. In embodiments, the disease associated with dysregulation and/or degeneration of dopaminergic neurons is amyotrophic lateral sclerosis. In embodiments, the disease associated with dysregulation and/or degeneration of dopaminergic neurons is schizophrenia. In embodiments, the disease associated with dysregulation and/or degeneration of dopaminergic neurons is drug addiction.

In embodiments, the disease associated with dysregulation and/or degeneration of dopaminergic neurons is a cancer. In embodiments, the disease associated with dysregulation and/or degeneration of dopaminergic neurons is an eye disease. In embodiments, the eye disease is cataract. In embodiments, the eye disease is congenital cataract.

In an aspect is provided a method of treating a neurodegenerative disease in a subject in need thereof, the method including administering to the subject in need thereof a therapeutically effective amount of a compound described herein, or a pharmaceutically acceptable salt thereof.

In embodiments, the disease is Parkinson's disease, Alzheimer's disease, multiple sclerosis, amyotrophic lateral sclerosis, schizophrenia, or drug addiction. In embodiments, the disease is Parkinson's disease. In embodiments, the disease is Alzheimer's disease. In embodiments, the disease is multiple sclerosis. In embodiments, the disease is amyotrophic lateral sclerosis. In embodiments, the disease is schizophrenia. In embodiments, the disease is drug addiction.

In an aspect is provided a method of treating a cancer in a subject in need thereof, the method including administering to the subject in need thereof a therapeutically effective amount of a compound described herein, or a pharmaceutically acceptable salt thereof.

In embodiments, the cancer is breast cancer, pancreatic cancer, bladder cancer, mucoepidermoid carcinoma, gastric cancer, prostate cancer, colorectal cancer, lung cancer, adrenocortical cancer, or cervical cancer.

In an aspect is provided a method of treating an eye disease in a subject in need thereof, the method including administering to the subject in need thereof a therapeutically effective amount of a compound described herein, or a pharmaceutically acceptable salt thereof. In embodiments, the eye disease is cataract. In embodiments, the eye disease is congenital cataract.

In an aspect is provided a method of reducing inflammation in a subject in need thereof, the method including administering to the subject in need thereof a therapeutically effective amount of a compound described herein, or a pharmaceutically acceptable salt thereof.

In embodiments, the method includes reducing inflammation in the central nervous system of the subject in need thereof.

In an aspect is provided a method of reducing oxidative stress in a subject in need thereof, the method including administering to the subject in need thereof a therapeutically effective amount of a compound described herein, or a pharmaceutically acceptable salt thereof.

In embodiments, the method includes reducing oxidative stress in the central nervous system of the subject in need thereof.

In an aspect is provided a method of modulating the level of activity of Nurr1 in a subject in need thereof, the method including administering to the subject in need thereof a therapeutically effective amount of a compound described herein, or a pharmaceutically acceptable salt thereof. In embodiments, the level of activity of Nurr1 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Nurr1 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Nurr1 in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Nurr1 in the subject is reduced by at least 1.5-, 2-, 3-, 4-5-, 6-, 7-, 8-, 9-, 10-, 15-, 20-, 25-, 30-, 35-, 40-, 45-, 50-, 60-, 70-, 80-, 90-, 100-, 150-, 200-250-, 300-, 350-, 400-, 450-, 500-, 600-, 700-, 800-, 900-, or 1000-fold.

In embodiments, the level of activity of Nurr1 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of Nurr1 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of Nurr1 in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of Nurr1 in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of differentiating a stem cell, the method including contacting the stem cell in vitro with a compound described herein, or a pharmaceutically acceptable salt thereof. In embodiments, the stem cell is differentiated to a dopaminergic neuron.

In an aspect is provided a method of increasing the level and/or activity of Nurr1 in a cell, the method including contacting the cell with a compound described herein, or a pharmaceutically acceptable salt thereof. In embodiments, the level and/or activity of Nurr1 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level and/or activity of Nurr1 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level and/or activity of Nurr1 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level and/or activity of Nurr1 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of reducing the level and/or activity of Nurr1 in a cell, the method including contacting the cell with a compound described herein, or a pharmaceutically acceptable salt thereof. In embodiments, the level and/or activity of Nurr1 in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level and/or activity of Nurr1 in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level and/or activity of Nurr1 in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level and/or activity of Nurr1 in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of TH in a subject in need thereof, the method including administering to the subject in need thereof a therapeutically effective amount of a compound described herein, or a pharmaceutically acceptable salt thereof. In embodiments, the level of activity of TH in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TH in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TH in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of TH in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of TH in a cell, the method including contacting the cell with a compound described herein, or a pharmaceutically acceptable salt thereof. In embodiments, the level of activity of TH in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TH in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TH in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of TH in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of DRD2 in a subject in need thereof, the method including administering to the subject in need thereof a therapeutically effective amount of a compound described herein, or a pharmaceutically acceptable salt thereof. In embodiments, the level of activity of DRD2 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DRD2 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DRD2 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of DRD2 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of DRD2 in a cell, the method including contacting the cell with a compound described herein, or a pharmaceutically acceptable salt thereof. In embodiments, the level of activity of DRD2 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DRD2 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DRD2 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of DRD2 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of VMAT2 in a subject in need thereof, the method including administering to the subject in need thereof a therapeutically effective amount of a compound described herein, or a pharmaceutically acceptable salt thereof. In embodiments, the level of activity of VMAT2 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of VMAT2 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of VMAT2 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of VMAT2 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of VMAT2 in a cell, the method including contacting the cell with a compound described herein. In embodiments, the level of activity of VMAT2 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of VMAT2 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of VMAT2 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of VMAT2 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of dopa decarboxylase (DDC) in a subject in need thereof, the method including administering to the subject in need thereof a therapeutically effective amount of a compound described herein, or a pharmaceutically acceptable salt thereof. In embodiments, the level of activity of DDC in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DDC in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DDC in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of DDC in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of dopa decarboxylase (DDC) in a cell, the method including contacting the cell with a compound described herein, or a pharmaceutically acceptable salt thereof. In embodiments, the level of activity of DDC in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DDC in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DDC in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of DDC in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of dopamine transporter (DAT) in a subject in need thereof, the method including administering to the subject in need thereof a therapeutically effective amount of a compound described herein, or a pharmaceutically acceptable salt thereof. In embodiments, the level of activity of DAT in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DAT in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DAT in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of DAT in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of dopamine transporter (DAT) in a cell, the method including contacting the cell with a compound described herein, or a pharmaceutically acceptable salt thereof. In embodiments, the level of activity of DAT in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DAT in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of DAT in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of DAT in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of BDNF in a subject in need thereof, the method including administering to the subject in need thereof a therapeutically effective amount of a compound described herein, or a pharmaceutically acceptable salt thereof. In embodiments, the level of activity of BDNF in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of BDNF in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of BDNF in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of BDNF in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of BDNF in a cell, the method including contacting the cell with a compound described herein, or a pharmaceutically acceptable salt thereof. In embodiments, the level of activity of BDNF in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of BDNF in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of BDNF in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of BDNF in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of NGF in a subject in need thereof, the method including administering to the subject in need thereof a therapeutically effective amount of a compound described herein, or a pharmaceutically acceptable salt thereof. In embodiments, the level of activity of NGF in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of NGF in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of NGF in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of NGF in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of NGF in a cell, the method including contacting the cell with a compound described herein, or a pharmaceutically acceptable salt thereof. In embodiments, the level of activity of NGF in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of NGF in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of NGF in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of NGF in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of GDNF receptor c-Ret in a subject in need thereof, the method including administering to the subject in need thereof a therapeutically effective amount of a compound described herein, or a pharmaceutically acceptable salt thereof. In embodiments, the level of activity of GDNF receptor c-Ret in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of GDNF receptor c-Ret in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of GDNF receptor c-Ret in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of GDNF receptor c-Ret in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of GDNF receptor c-Ret in a cell, the method including contacting the cell with a compound described herein, or a pharmaceutically acceptable salt thereof. In embodiments, the level of activity of GDNF receptor c-Ret in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of GDNF receptor c-Ret in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of GDNF receptor c-Ret in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of GDNF receptor c-Ret in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of SOD1 in a subject in need thereof, the method including administering to the subject in need thereof a therapeutically effective amount of a compound described herein, or a pharmaceutically acceptable salt thereof. In embodiments, the level of activity of SOD1 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of SOD1 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of SOD1 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of SOD1 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of SOD1 in a cell, the method including contacting the cell with a compound described herein, or a pharmaceutically acceptable salt thereof. In embodiments, the level of activity of SOD1 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of SOD1 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of SOD1 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of SOD1 in the cell is increased by at least 1.5-, 2-, 3-, 4-, 5-, 6-, 7-, 8-, 9-, 10-, 15-, 20-, 25-, 30-35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of reducing the level of activity of TNFα in a subject in need thereof, the method including administering to the subject in need thereof a therapeutically effective amount of a compound described herein, or a pharmaceutically acceptable salt thereof. In embodiments, the level of activity of TNFα in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TNFα in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TNFα in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of TNFα in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of reducing the level of activity of TNFα in a cell, the method including contacting the cell with a compound described herein, or a pharmaceutically acceptable salt thereof. In embodiments, the level of activity of TNFα in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TNFα in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of TNFα in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of TNFα in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of reducing the level of activity of iNOS in a subject in need thereof, the method including administering to the subject in need thereof a therapeutically effective amount of a compound described herein, or a pharmaceutically acceptable salt thereof. In embodiments, the level of activity of iNOS in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of iNOS in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of iNOS in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of iNOS in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of reducing the level of activity of iNOS in a cell, the method including contacting the cell with a compound described herein, or a pharmaceutically acceptable salt thereof. In embodiments, the level of activity of iNOS in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of iNOS in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of iNOS in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of iNOS in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of reducing the level of activity of IL-1β in a subject in need thereof, the method including administering to the subject in need thereof a therapeutically effective amount of a compound described herein, or a pharmaceutically acceptable salt thereof. In embodiments, the level of activity of IL-1β in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of IL-1β in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of IL-1βR in the subject is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of IL-1β in the subject is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of reducing the level of activity of IL-1β in a cell, the method including contacting the cell with a compound described herein, or a pharmaceutically acceptable salt thereof. In embodiments, the level of activity of IL-1β in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of IL-1β in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of IL-1β in the cell is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of IL-1β in the cell is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of Pitx3 in a subject in need thereof, the method including administering to the subject in need thereof a therapeutically effective amount of a compound described herein, or a pharmaceutically acceptable salt thereof. In embodiments, the level of activity of Pitx3 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Pitx3 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Pitx3 in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of Pitx3 in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In an aspect is provided a method of increasing the level of activity of Pitx3 in a cell, the method including contacting the cell with a compound described herein, or a pharmaceutically acceptable salt thereof. In embodiments, the level of activity of Pitx3 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Pitx3 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of activity of Pitx3 in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of Pitx3 in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the method includes increasing the level of dopamine in a subject in need thereof, the method including administering to the subject in need thereof a therapeutically effective amount of a compound described herein. In embodiments, the level of dopamine in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of dopamine in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of dopamine in the subject is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of dopamine in the subject is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

In embodiments, the method includes increasing the level of dopamine in a cell, the method including contacting the cell with a compound described herein. In embodiments, the level of dopamine in the cell is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of dopamine in the cell is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing synthesis of dopamine in a cell as compared to a control (e.g., absence of the compound). In embodiments, the level of synthesis of dopamine is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-600-, 700-, 800-, 900-, or 1000-fold. In embodiments, the level of synthesis of dopamine is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing packaging of dopamine in a cell as compared to a control (e.g., absence of the compound). In embodiments, the level of packaging of dopamine is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of packaging of dopamine is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing reuptake of dopamine in a cell as compared to a control (e.g., absence of the compound). In embodiments, the level of reuptake of dopamine is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of reuptake of dopamine is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing development of dopaminergic neurons as compared to a control (e.g., absence of the compound). In embodiments, the level of development of dopaminergic neurons is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of development of dopaminergic neurons is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing maintenance of dopaminergic neurons as compared to a control (e.g., absence of the compound). In embodiments, the level of maintenance of dopaminergic neurons is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of maintenance of dopaminergic neurons is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes increasing survival of dopaminergic neurons as compared to a control (e.g., absence of the compound). In embodiments, the level of survival of dopaminergic neurons is increased by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the level of survival of dopaminergic neurons is increased by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the method includes binding Nurr1 (e.g., human Nurr1) with a compound described herein. In embodiments, the method includes noncovalently binding Nurr1 (e.g., human Nurr1) with a compound described herein.

In embodiments, the method includes contacting an amino acid corresponding to Arg515 of human Nurr1 with a compound described herein. In embodiments, the method includes contacting an amino acid corresponding to Arg563 of human Nurr1 with a compound described herein. In embodiments, the method includes contacting an amino acid corresponding to Glu445 of human Nurr1 with a compound described herein.

In embodiments, the method includes stabilizing a Nurr1 monomer with a compound described herein. In embodiments, the Nurr1 monomer is stabilized by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold as compared to a control (e.g., absence of the compound). In embodiments, the Nurr1 monomer is stabilized by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold as compared to a control (e.g., absence of the compound).

In embodiments, the method includes stabilizing a Nurr1 homodimer with a compound described herein. In embodiments, the Nurr1 homodimer is stabilized by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold as compared to a control (e.g., absence of the compound). In embodiments, the Nurr1 homodimer is stabilized by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold as compared to a control (e.g., absence of the compound).

In embodiments, the method includes stabilizing a head-to-tail Nurr1 homodimer with a compound described herein. In embodiments, the head-to-tail Nurr1 homodimer is stabilized by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold as compared to a control (e.g., absence of the compound). In embodiments, the head-to-tail Nurr1 homodimer is stabilized by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold as compared to a control (e.g., absence of the compound).

In embodiments, the method includes stabilizing a Nurr1 heterodimer with a compound described herein. In embodiments, the Nurr1 heterodimer is stabilized by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold as compared to a control (e.g., absence of the compound). In embodiments, the Nurr1 heterodimer is stabilized by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold as compared to a control (e.g., absence of the compound). In embodiments, the Nurr1 heterodimer is a heterodimer with RXRα.

In embodiments, the method includes contacting a Nurr1 monomer with a compound described herein. In embodiments, the method includes contacting a Nurr1 homodimer with a compound described herein. In embodiments, the method includes contacting a head-to-tail Nurr1 homodimer with a compound described herein. In embodiments, the method includes contacting a Nurr1 heterodimer with a compound described herein. In embodiments, the Nurr1 heterodimer is a heterodimer with RXRα.

In embodiments, the method includes binding a Nurr1 monomer with a compound described herein. In embodiments, the method includes binding a Nurr1 homodimer with a compound described herein. In embodiments, the method includes binding a head-to-tail Nurr1 homodimer with a compound described herein. In embodiments, the method includes binding a Nurr1 heterodimer with a compound described herein. In embodiments, the Nurr1 heterodimer is a heterodimer with RXRα.

In embodiments, the method includes precluding the formation of Nurr1:RXR heterodimers with a compound described herein.

In embodiments, the method includes stabilizing a Nurr1 dimer conformation wherein the distance between the N-termini is about 74.0 Å with a compound described herein. In embodiments, the method includes stabilizing a Nurr1 dimer conformation wherein the distance between the N-termini is at least 74.0 Å with a compound described herein. In embodiments, the method includes stabilizing a Nurr1 dimer conformation wherein the distance between the N-termini is less than 74.0 Å with a compound described herein.

In embodiments, the method includes contacting a Nurr1 dimer conformation wherein the distance between the N-termini is about 74.0 Å with a compound described herein. In embodiments, the method includes contacting a Nurr1 dimer conformation wherein the distance between the N-termini is at least 74.0 Å with a compound described herein. In embodiments, the method includes contacting a Nurr1 dimer conformation wherein the distance between the N-termini is less than 74.0 Å with a compound described herein.

In embodiments, the method includes binding a Nurr1 dimer conformation wherein the distance between the N-termini is about 74.0 Å with a compound described herein. In embodiments, the method includes binding a Nurr1 dimer conformation wherein the distance between the N-termini is at least 74.0 Å with a compound described herein. In embodiments, the method includes binding a Nurr1 dimer conformation wherein the distance between the N-termini is less than 74.0 Å with a compound described herein.

In embodiments, the method includes stabilizing a Nurr1 dimer conformation wherein the distance between the N-termini is about 59.3 Å with a compound described herein. In embodiments, the method includes stabilizing a Nurr1 dimer conformation wherein the distance between the N-termini is at least 59.3 Å with a compound described herein. In embodiments, the method includes stabilizing a Nurr1 dimer conformation wherein the distance between the N-termini is less than 59.3 Å with a compound described herein.

In embodiments, the method includes contacting a Nurr1 dimer conformation wherein the distance between the N-termini is about 59.3 Å with a compound described herein. In embodiments, the method includes contacting a Nurr1 dimer conformation wherein the distance between the N-termini is at least 59.3 Å with a compound described herein. In embodiments, the method includes contacting a Nurr1 dimer conformation wherein the distance between the N-termini is less than 59.3 Å with a compound described herein.

In embodiments, the method includes binding a Nurr1 dimer conformation wherein the distance between the N-termini is about 59.3 Å with a compound described herein. In embodiments, the method includes binding a Nurr1 dimer conformation wherein the distance between the N-termini is at least 59.3 Å with a compound described herein. In embodiments, the method includes binding a Nurr1 dimer conformation wherein the distance between the N-termini is less than 59.3 Å with a compound described herein.

In embodiments, the method includes binding a Nurr1 and inducing Nurr1 binding to a NBRE, a NuRE, or a DR-5 response element. In embodiments, the method includes binding a Nurr1 and inducing Nurr1 binding to a NBRE. In embodiments, the method includes binding a Nurr1 and inducing Nurr1 binding to a NuRE. In embodiments, the method includes binding a Nurr1 and inducing Nurr1 binding to a DR-5 response element.

### V. Embodiments

Embodiment P1. A compound having the formula

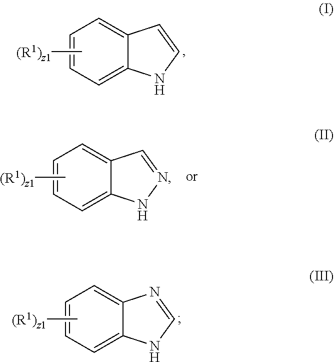

wherein
R¹ is independently halogen, —CX¹₃, —CHX¹₂, —CH₂X¹, —OCX¹₃, —OCH₂X¹, —OCHX¹₂, —CN, —SO 1R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SF₅, —SSR^{1D}, —SiR^{1A}R^{1B}R^{1C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A}, R^{1B}, R^{1C}, and R^{1D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃,—OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may be joined to form a substituted or unsubstituted heterocycloalkyl or substituted or unsubstituted heteroaryl;
n1 is independently an integer from 0 to 4;
m1 and v1 are independently 1 or 2;
X¹ is independently —F, —Cl, —Br, or —I; and
z1 is an integer from 0 to 6.

Embodiment P2. The compound of embodiment P1, wherein the compound is not

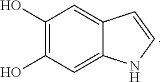 [SMILES: Oc1cc2cc[nH]c2cc1O]

Embodiment P3. The compound of one of embodiments P1 to P2, wherein R¹ is independently —F, —Cl, —Br, or —I.

Embodiment P4. The compound of one of embodiments P1 to P2, wherein the compound has the formula

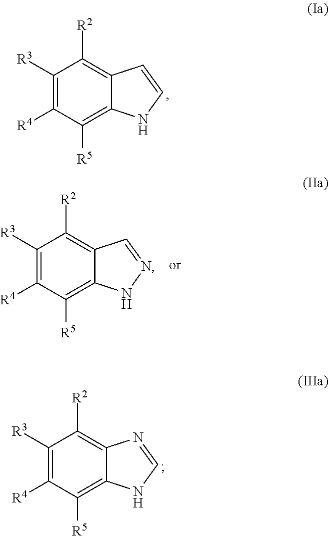

wherein
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —SC(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, —SF₅, —SSR^{2D}, —SiR^{2A}R^{2B}R^{2C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —SC(O)R^{3C}, —C(O)OR^{3C}, —C(O)NR^{3A}R^{3B}, —OR^{3D}, —SR^{3D}, —SeR^{3D}, —N^{3A}SO₂R^{3D}, —NR^{3A}C(O)R^{3C}, —NR^{3A}C(O)OR^{3C}, —NR^{3A}OR^{3C}, —N₃, —SF₅, —SSR^{3D}, —SiR^{3A}R^{3B}R^{3C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —SC(O)R^{4C}, —C(O)OR^{4C}, —C(O)NR^{4A}R^{4B}, —OR^{4D}, —SR^{4D}, —SeR^{4D}, —NR^{4A}SO_{24D}, —NR^{4A}C(O)R^{4C}, —NR^{4A}C(O)OR^{4C}, —NR^{4A}OR^{4C}, —N₃, —SF₅, —SSR^{4D}, —SiR^{4A}R^{4B}R^{4C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —SC(O)R^{5C}, —C(O)OR^{5C}, —C(O)NR^{5A}R^{5B}, —OR^{5D}, —SR^{5D}, —SeR^{5D}, —NR^{5A}SO₂R^{5D}, —NR^{5A}C(O)R^{5C}, —NR^{5A}C(O)OR^{5C}, —NR^{5A}OR^{5C}, —N₃, —SF₅, —SSR^{5D}, —SiR^{5A}R^{5B}R^{5C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl; R^{3A} and R^{3B} substituents bonded to the same nitrogen atom may be joined to form a substituted or unsubstituted heterocycloalkyl or substituted or unsubstituted heteroaryl; R^{4A} and R^{4B} substituents bonded to the same nitrogen atom may be joined to form a substituted or unsubstituted heterocycloalkyl or substituted or unsubstituted heteroaryl; R^{5A} and R^{5B} substituents bonded to the same nitrogen atom may be joined to form a substituted or unsubstituted heterocycloalkyl or substituted or unsubstituted heteroaryl;
n2, n3, n4, and n5 are independently an integer from 0 to 4;
m2, m3, m4, m5, v2, v3, v4, and v5 are independently 1 or 2; and
X², X³, X⁴, and X⁵ are independently —F, —Cl, —Br, or —I.

Embodiment P5. The compound of embodiment P4, wherein the compound has the formula

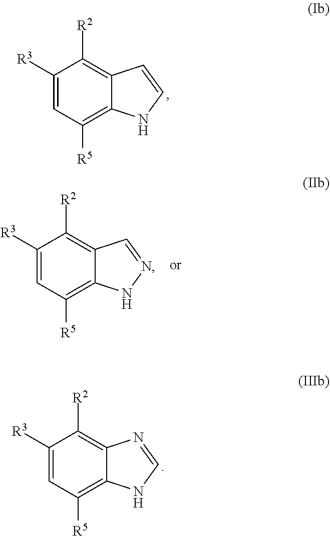

Embodiment P6. The compound of one of embodiments P4 to P5, wherein R³ is halogen.

Embodiment P7. The compound of embodiment P6, wherein R³ is —Br or —Cl.

Embodiment P8. The compound of one of embodiments P4 to P7, wherein R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P9. The compound of one of embodiments P4 to P7, wherein R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P10. The compound of one of embodiments P4 to P7, wherein R² is hydrogen, halogen, —CF₃, —CH₂F, —CHF₂, —CN, —OH, —NH₂, —COOH, —CONH₂, —OCF₃, —OCHF₂, —OCH₂F, substituted or unsubstituted C₁-C₄ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

Embodiment P11. The compound of one of embodiments P4 to P10, wherein R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P12. The compound of one of embodiments P4 to P10, wherein R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P13. The compound of one of embodiments P4 to P10, wherein R⁵ is hydrogen, halogen, —CF₃, —CH₂F, —CHF₂, —CN, —OH, —NH₂, —COOH, —CONH₂, —OCF₃, —OCHF₂, —OCH₂F, substituted or unsubstituted C₁-C₄ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

Embodiment P14. A pharmaceutical composition comprising a compound of one of embodiments P1 to P13, or a pharmaceutically acceptable salt thereof, and a pharmaceutically acceptable excipient.

Embodiment P15. A method of treating a disease associated with dysregulation and/or degeneration of dopaminergic neurons in the central nervous system of a subject in need thereof, said method comprising administering to the subject in need thereof a therapeutically effective amount of a compound of one of embodiments P1 to P13, or a pharmaceutically acceptable salt thereof.

Embodiment P16. The method of embodiment P15, wherein said disease associated with dysregulation and/or degeneration of dopaminergic neurons is Parkinson's disease, Alzheimer's disease, multiple sclerosis, amyotrophic lateral sclerosis, schizophrenia, or drug addiction.

Embodiment P17. The method of embodiment P15, wherein said disease associated with dysregulation and/or degeneration of dopaminergic neurons is Parkinson's disease.

Embodiment P18. A method of treating a cancer in a subject in need thereof, the method comprising administering to the subject in need thereof a therapeutically effective amount of a compound of one of embodiments P1 to P13, or a pharmaceutically acceptable salt thereof.

Embodiment P19. The method of embodiment P18, wherein said cancer is breast cancer, pancreatic cancer, bladder cancer, mucoepidermoid carcinoma, gastric cancer, prostate cancer, colorectal cancer, lung cancer, adrenocortical cancer, or cervical cancer.

Embodiment P20. A method of modulating the level of activity of Nurr1 in a subject in need thereof, the method comprising administering to the subject in need thereof an effective amount of a compound of one of embodiments P1 to P13, or a pharmaceutically acceptable salt thereof.

Embodiment P21. A method of increasing the level and/or activity of Nurr1 in a cell, the method comprising contacting said cell with a compound of one of embodiments P1 to P13, or a pharmaceutically acceptable salt thereof.

Embodiment P22. A method of increasing the level of dopamine in a cell, the method comprising contacting said cell with a compound of one of embodiments P1 to P13, or a pharmaceutically acceptable salt thereof.

Embodiment P23. A method of differentiating a stem cell, the method comprising contacting said stem cell in vitro with a compound of one of embodiments P1 to P13, or a pharmaceutically acceptable salt thereof.

Embodiment P24. The method of embodiment P23, wherein said stem cell is differentiated to a dopaminergic neuron.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Nurr1 (NR⁴A2) Receptor Modulators

We have identified compounds that bind directly to and stimulate the activity of nuclear receptor related-1 protein (Nurr1), also known as NR⁴A2, a transcription factor regarded as a potential therapeutic target for Parkinson's disease, as well as other disorders associated with the dysregulation and degeneration of dopaminergic neurons (e.g., multiple sclerosis, amyotrophic lateral sclerosis, schizophrenia, drug addiction).

Because Nurr1 μlays an essential role in regulating dopamine homeostasis (i.e., controlling expression of the genes required for the synthesis, packaging, and re-uptake of dopamine), it might be directly regulated by the neurotransmitter itself, or one of its metabolites. This led us to investigate the binding affinity of a dopamine, L-DOPA, 5,6-dihydroxyindole, and 5,6-dihydroxyindole carboxylic acid for the Nurr1 LBD. We discovered that 5,6-dihydroxyindole (DHI) binds directly to and stimulates the activity of Nurr1. These data, along with a crystal structure of DHI covalently bound to the Nurr1 ligand binding domain were published in 2019 (Bruning et al., Cell 2019).

DHI is an unstable molecule, auto-oxidizing and polymerizing in solution to form a chromogenic pigment, and in the brain to form neuromelanin. Thus, it is unsuitable for robust biological studies. We therefore sought to identify stable analogs of DHI that would also bind to and activate the receptor in cells. We started with ˜20 analogs and measured the binding affinity for the Nurr1 ligand binding domain in vitro, using microscale thermophoresis, and the activity against the full length receptor in cells, using qPCR to measure the expression of three Nurr1 target genes (Th, Vmat2, Nurr1). We also performed computational analyses to learn more about specific ligand-receptor interactions important for binding and activity of this series of compounds. These data are summarized in FIG. 1.

We found that 5-chloroindole and 5-bromoindole are effective Nurr1 agonists, upregulating the expression of the Nurr1 target genes tyrosine hydroxylase (Th) and vesicular monoamine transporter (Vmat2) in MN9D cells, a cell line derived from dopaminergic neurons which endogenously expresses full length Nurr1 and its target genes. These compounds bind with micromolar affinity, but have very good ligand efficiency due to their small size (i.e., low molecular weight). Next, we will build on these findings to find additional analogs with improved efficacy and affinity.

Modulators of Nurr1 receptor activity have potential applications for the treatment of diseases associated with the dysregulation and/or degeneration of dopaminergic neurons in the central nervous system. These diseases include Parkinson's disease, multiple sclerosis, amyotrophic lateral sclerosis, schizophrenia, and drug addiction. Our efforts are currently focused on developing Nurr1 modulators to treat the symptoms and progression of Parkinson's Disease (PD).

Current therapeutics for Parkinson's disease (PD) are symptom-modifying only, have no effect on disease progression, and lose efficacy over time. Existing therapies for PD relieve symptoms by increasing dopamine levels in the central nervous system by increasing levels of dopamine's biosynthetic precursor (L-DOPA), inhibiting the breakdown of dopamine (monoamine oxidase inhibitors), or by bypassing dopamine itself (dopamine receptor agonists).

Small molecule modulators of Nurr1 function may be used to (1) stimulate the development of dopaminergic neurons from stem cells, (2) support the health of mature dopaminergic neurons, (3) prevent the degeneration of mature dopaminergic neurons, (4) stimulate the synthesis of dopamine in neurons. Diseases that would be impacted by these functions include Parkinson's disease, multiple sclerosis, amyotrophic lateral sclerosis, schizophrenia, and drug addiction.

A handful of putative Nurr1 agonists have been reported in the patent and scientific literature (reviewed in Dong et al., 2016). With the exception of amodiaquine (Kim et al., 2015), there is little evidence that any of these compounds bind directly to Nurr1. Our invention identifies ligands that both bind directly to the Nurr1 and modulate Nurr1 transcriptional activity of the receptor in cells.

TABLE 1
Measured affinities and amplitude changes observed for indicated
compounds.

| | | | K_{D} | | Amplitude |
|---|---|---|---|---|---|
| Compound | Nurr1 Variant | K_{D} (μM) | (% change) | Amplitude | (% change) |
| 5,6-dibromoindole | WT | 2.2 + 0.2 | — | 31 + 1 | — |
| | H516 | 6.3 + 1 | +186% | 35 + 2 | +13% |
| | R563A | 11 + 3 | +400% | 32 + 3 | +3% |
| | H516A/R563A | 16 + 4 | +627% | 43 + 5 | +39% |
| 5-bromoindole | WT | 10 + 2 | — | 72 + 8 | 0 |
| | H516 | 11 + 3 | +10% | 41 + 5 | −43% |
| | R563A | NA | NA | ≤10 | ≥−86% |
| | H516A/R563A | - NA | NA | - (≤10) | ≥−86% |
| 5,6-dichloroindole | WT | 4.5 + 0.9 | — | 54 + 4 | — |
| | H516 | 12 + 3 | +166% | 67 + 9 | +19% |
| | R563A | 13 + 2 | +189% | 47 + 3 | −13% |
| | H516A/R563A | 18 + 4 | +304% | 54 + 5 | 0% |
| 5-chloroindole | WT | 25 + 4 | — | 68 + 4 | — |
| | H516 | 16 + 4 | −36% | 34 + 4 | −50% |
| | R563A | 13 + 4 | −48% | 23 + 2 | −66% |
| | H516A/R563A | 8.0 + 2 | −68% | 26 + 2 | −62% |

### REFERENCES FOR EXAMPLE 1

1. Bruning, J. M., Wang, Y., Oltrabella, F., Tian, B., Kholodar, S. A., Liu, H., Bhattacharya, P., Guo, S., Holton, J. M., Fletterick, R. J., Jacobson, M. P., England, P. A. (2019) Cell Chem. Biol. 26(5): 674-685. 2. Dong, J., Li, S., Mo, J. L., Cai, H. B., and Le, W. D. (2016). CNS Neurosci Ther 22, 351-359. 3. Kim, C. H., Han, B. S., Moon, J., Kim, D. J., Shin, J., Rajan, S., Nguyen, Q. T., Sohn, M., Kim, W. G., Han, M., et al. (2015). Proc Natl Acad Sci USA 112, 8756-8761.

### Example 2: Analogs of the Dopamine Metabolite 5,6-Dihydroxyindole Bind Directly to and Activate the Nuclear Receptor Nurr1 (NR⁴A2)

The nuclear receptor Nurr1 (NR⁴A2) plays critical roles in both developing and adult midbrain dopaminergic neurons, controlling the transcription of genes required for the synthesis (TH) and vesicular packaging (VMAT2) of dopamine, among other essential biological functions (e.g., management of oxidative stress, responsiveness to inflammatory signals).¹⁻³ Clinical and experimental data indicate that disrupted Nurr1 function contributes to inducing the dysregulation of dopaminergic neurons observed in the early stages of Parkinson's disease (PD), as well as other dopamine-related CNS disorders (e.g., ALS, SCZ).^{4.15} Unraveling the complex biology of Nurr1 requires bona fide Nurr1-targeting synthetic small molecules that can be used to directly interrogate the receptor. Phenotypic assays have identified synthetic ligands that reportedly up-regulate transcription and protein levels of Nurr1 target genes, provide some degree of neuroprotection, and improve behavioral deficits in mouse models.¹⁶⁻²² However, there is little evidence that these compounds directly activate endogenous Nurr1, with the exception of the antimalarial drug amodiaquine and related analogs.^{17,23,24}

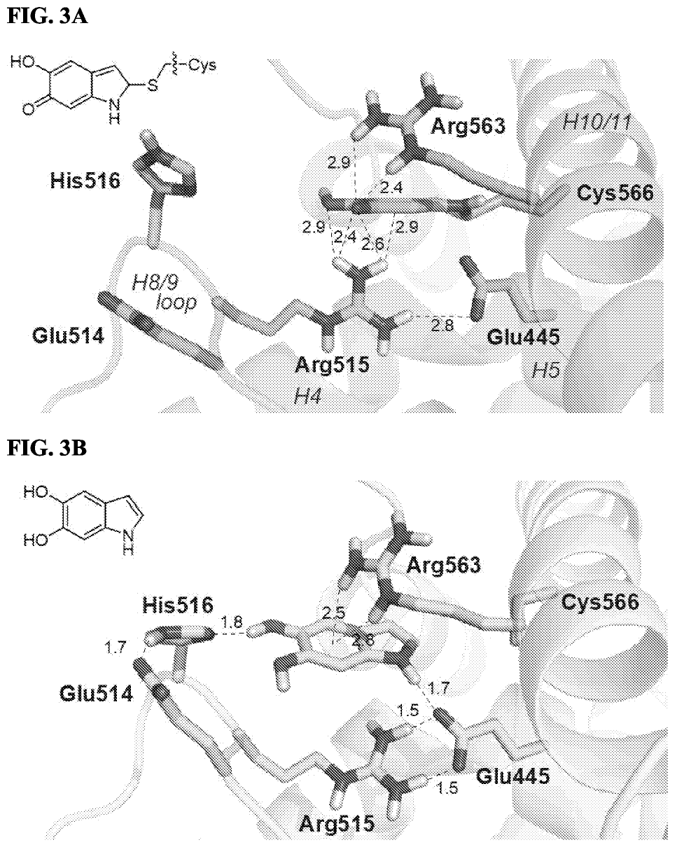

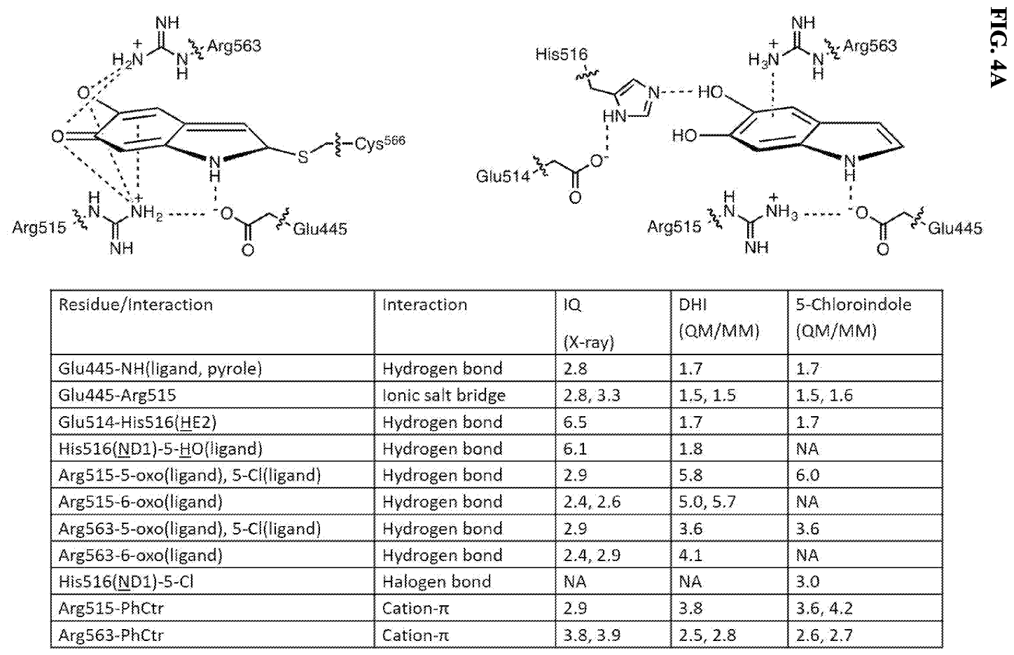

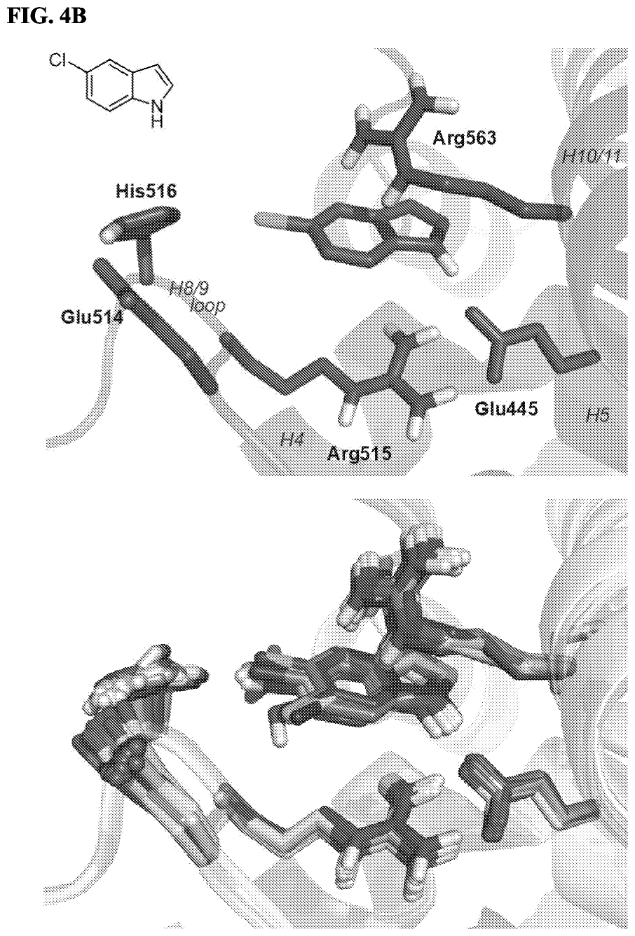

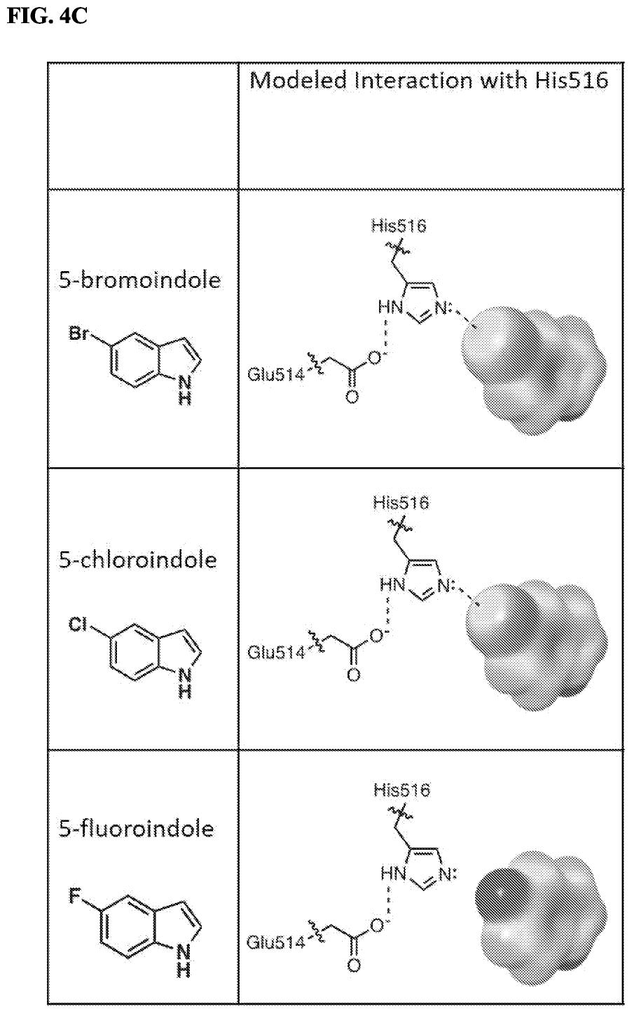

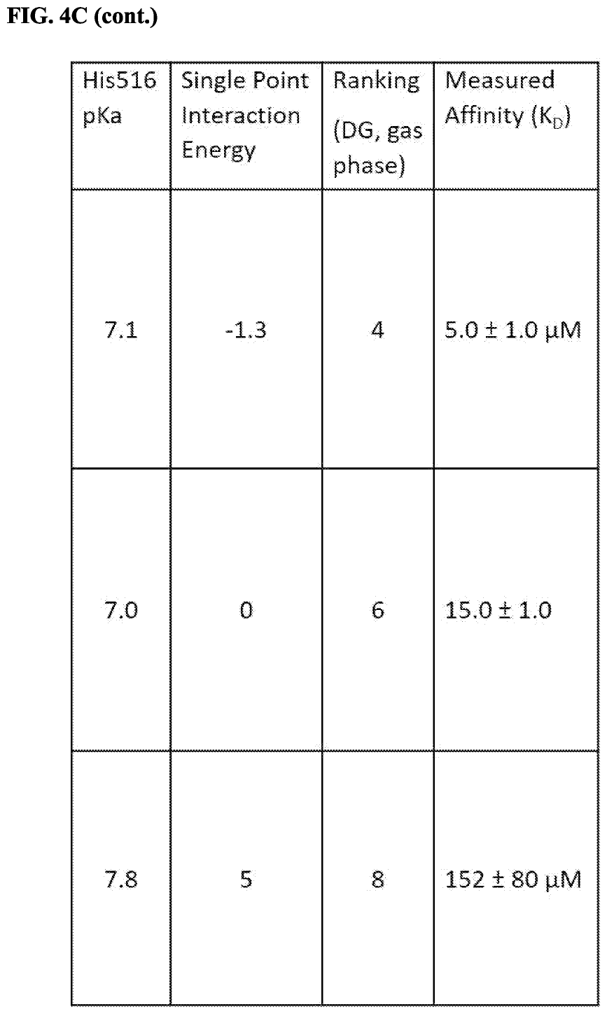

We demonstrated that the endogenous dopamine metabolite 5,6-dihydroxyindole (DHI) stimulates the expression of th and vmat2 in zebrafish and binds to the Nurr1 ligand binding domain (LBD) within a non-canonical ligand binding pocket, forming a reversible covalent adduct with the side chain of Cys566, likely the result of a Michael addition to the oxidized, indolequinone (IQ) form of DHI (FIGS. 3A-3B, FIG. 4A).²⁵ The term “canonical ligand binding pocket” refers to the expected site of ligand binding based on the classic binding pocket for endogenous ligands in well-characterized nuclear receptors, such as androgen, estrogen, and glucocorticoid receptors. An endogenous prostaglandin (PGA1) has subsequently been shown to partially occupy this site, and form a covalent adduct with Cys566.²⁶ DHI is unsuitable for robust biological studies, however, as it readily auto-oxidizes and polymerizes with itself and other molecules, in solution to form a chromogenic pigment and, in neurons to form neuromelanin.²⁷⁻²⁹ The combination of the enamine moiety on the pyrrole unit and the 5,6-dihydroxy substitution on the six membered ring results in a unique π electron system that renders DHI remarkably reactive. We, therefore, set out to identify unreactive analogs of DHI.

Previous biophysical and theoretical studies of indole (e.g., tryptophan analogs) interactions with cations established the importance of cation-π interactions in molecular recognition by proteins.^{31, 32} The crystal structure of the Nurr1-IQ complex shows a cation-71 interaction between side chain of Arg515 and the IQ adduct (FIG. 3A). Weak electron density for the Arg563 side chain suggests this residue is dynamic, and poised to form a second cation-π interaction with the ligand. Indeed, in quantum mechanical models of DHI bound non-covalently within the same “566 site,” a cation-π interaction with Arg563 appears important for stabilizing the aromatic indole system (FIG. 3B). To identify unreactive analogs of DHI, we systematically replaced the 5- and 6-hydroxyl groups on the indole with a series of substituents expected to impact the strength of these cation-π interactions, and measured ligand affinities for the LBD in vitro, and activities in cells.

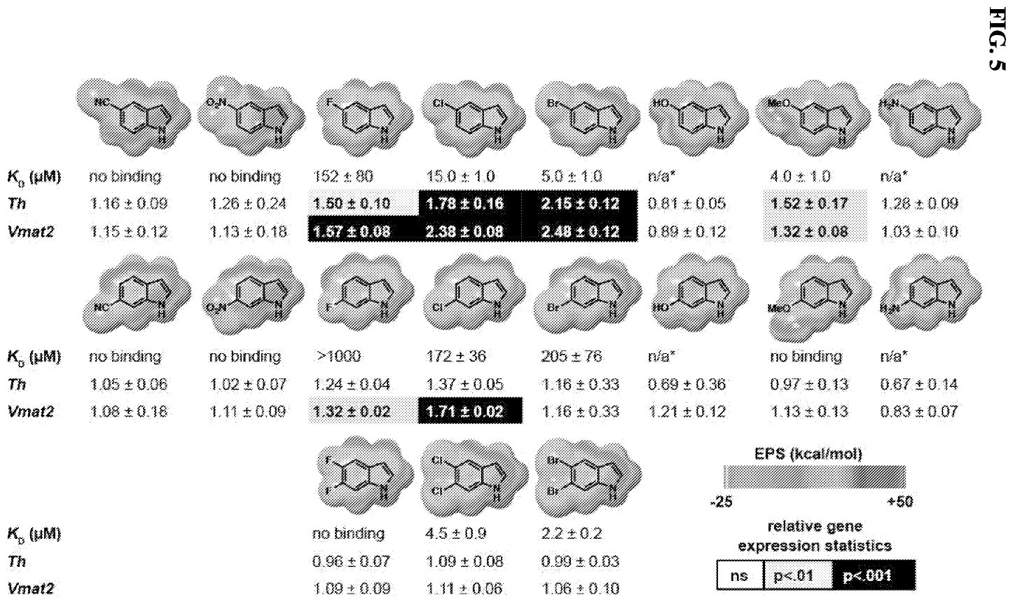

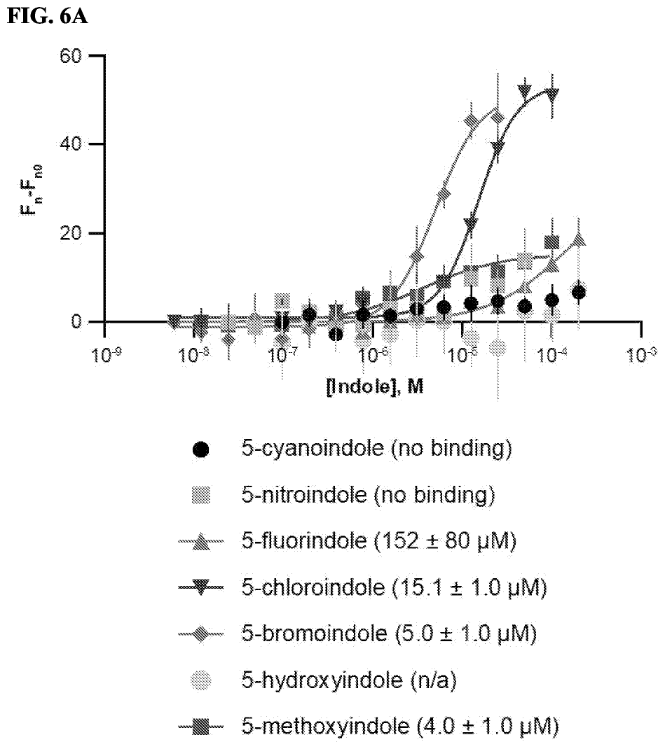

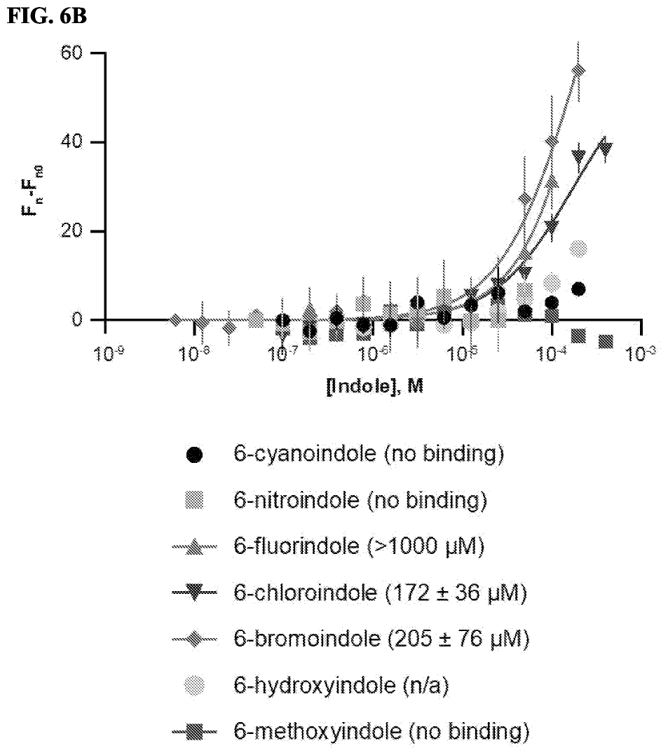

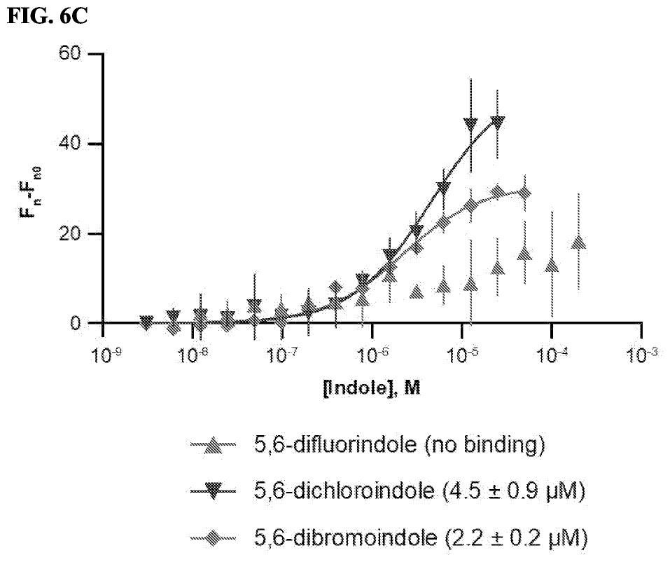

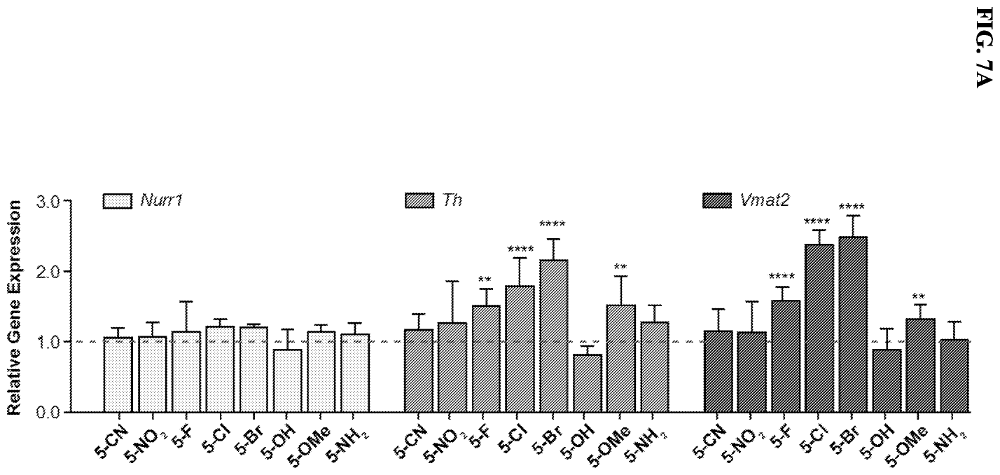

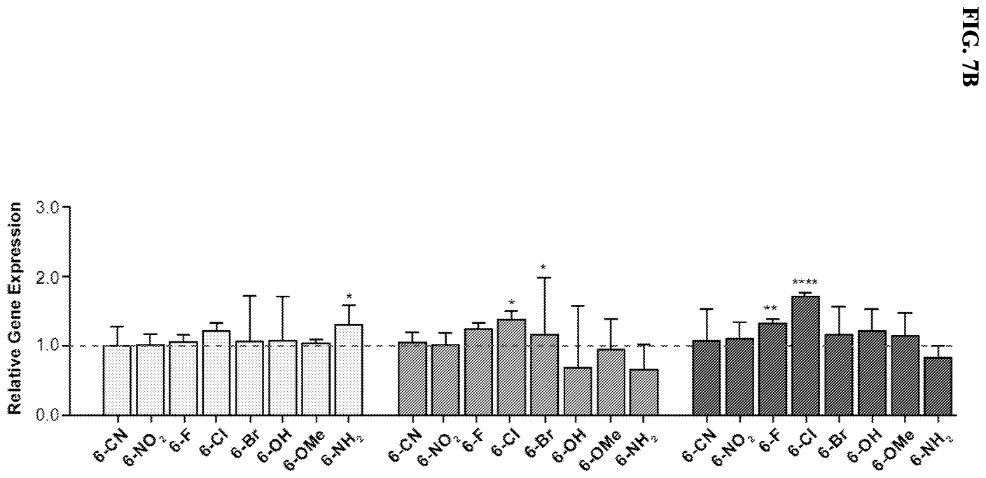

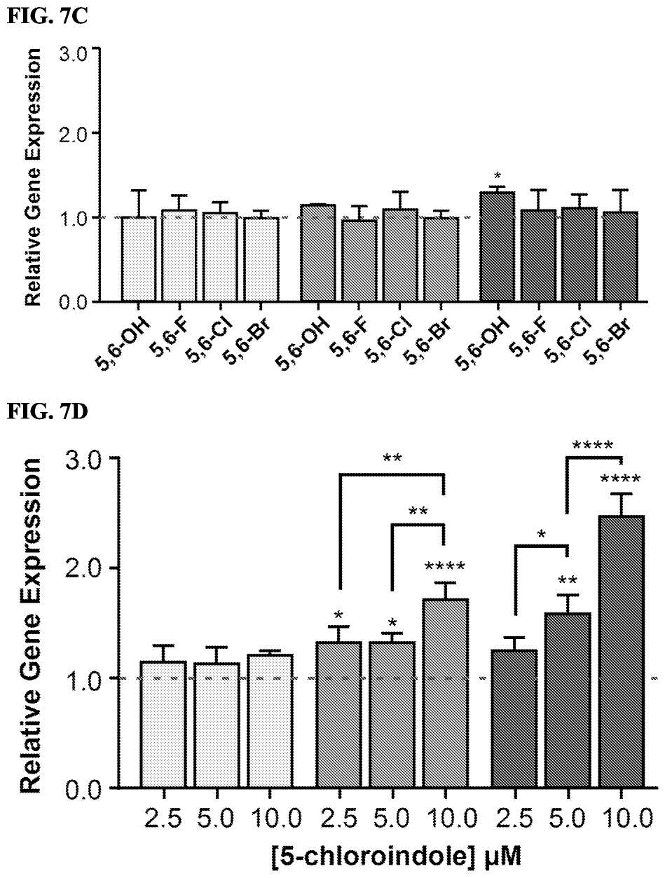

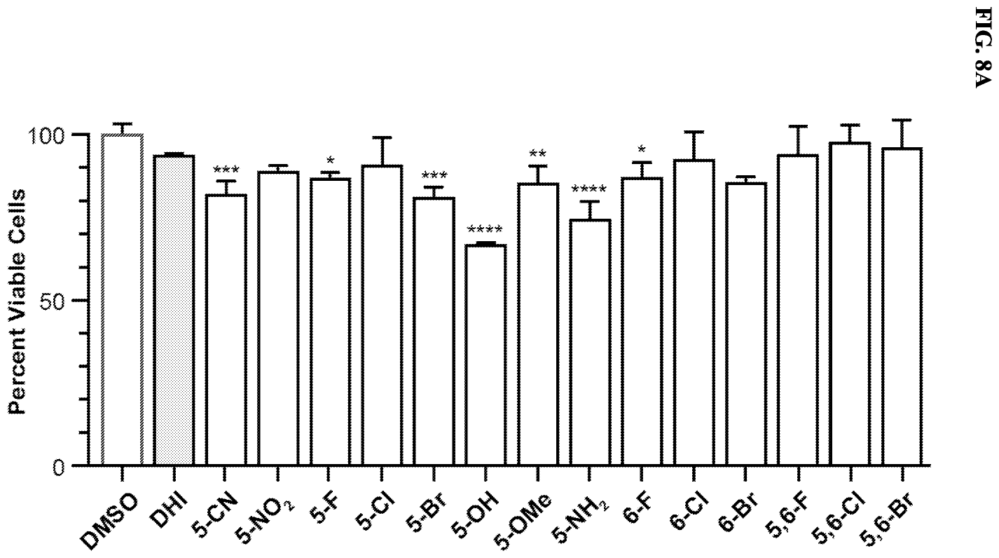

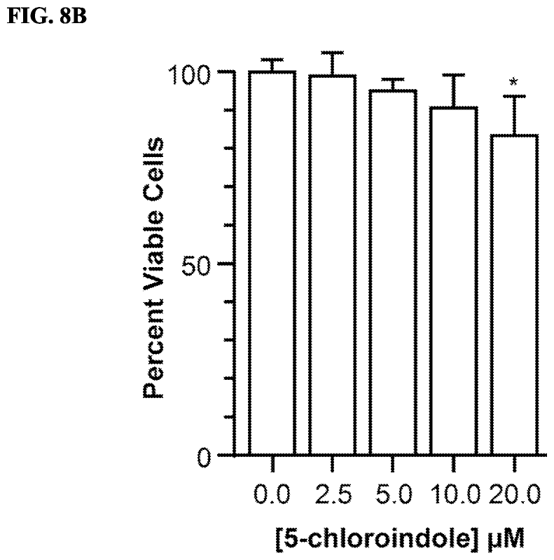

Approximately 20 substituted indoles, each predicted to bind within the LBD with poses nearly identical to DHI (FIG. 4B), were purchased from commercial suppliers and, for each compound, we determined (1) the molecular electrostatic potential (ESP) surface using density functional theory, (2) the affinity for the Nurr1 LBD using microscale thermophoresis (MST), (3) the activity against the full-length receptor using qPCR of Nurr1 target gene transcripts in MN9D cells, and (4) the cytotoxicity in MN9D cells. The affinity and activity across the entire series of indoles reveal that only indoles with a negative ESP surface, and thus capable of forming a cation-π interaction with the protein, exhibit saturable binding to the Nurr1 LBD (FIG. 5, FIGS. 6A-6C). The MN9D cell line, a fusion of embryonic ventral mesencephalic and neuroblastoma cells, is extensively used as a model of dopamine neurons because it expresses tyrosine hydroxylase and synthesizes and releases dopamine. Dissociation constants could not be obtained for several indoles with negative ESP surfaces, expected to bind tightly to Nurr1, owing to their chemical instability in solution.³³⁻³⁵ Nonetheless, trends in the binding affinities among all of the halogenated indoles tested (K_{D}: Br<C₁<<F) are consistent with a previous reports surrounding the relative strength of cation-π interactions involving substituted indoles.^{31, 32} Intriguingly, the data also reveal that among the indoles that bind to the LBD, only a subset also stimulate the transcription of Nurr1 target genes. Whereas 5-chloro and 5-bromoindole bind with micromolar affinity (K_{D}=15 μM and 5 μM, respectively) and increase the expression of both Th (1.8- and 2.2-fold) and Vmat2 (2.4- and 2.5-fold) in MN9D cells, the corresponding dihalogenated indoles bind with comparable affinity to the LBD, but do not modulate the expression of either gene. Cytotoxicity assays show that 5-chloroindole is not cytotoxic, whereas 5-bromoindole is among several indoles tested that are somewhat toxic to cells under certain conditions (≥10 μM, 24 h) (FIGS. 8A-8B).

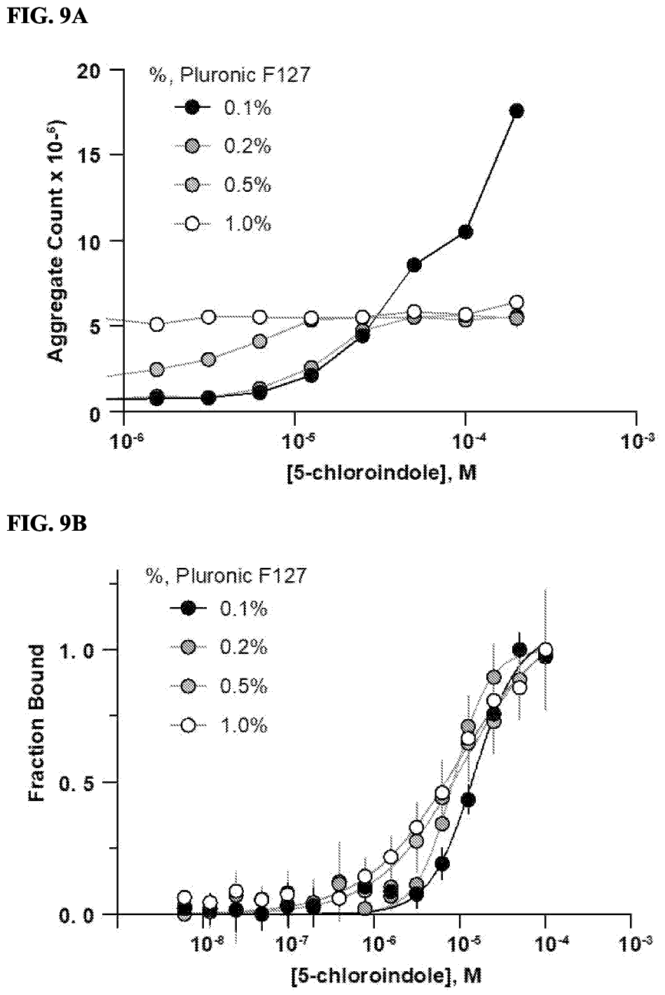

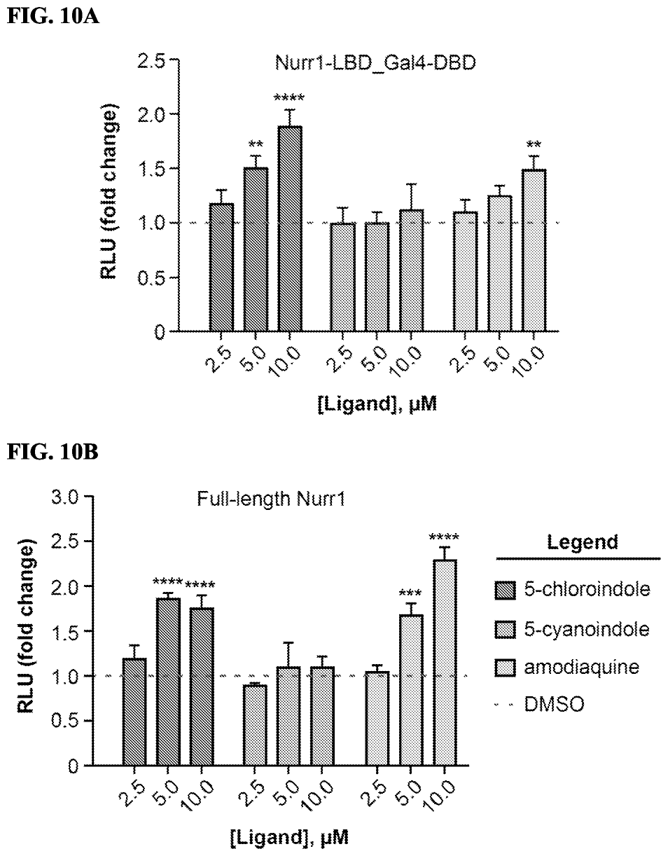

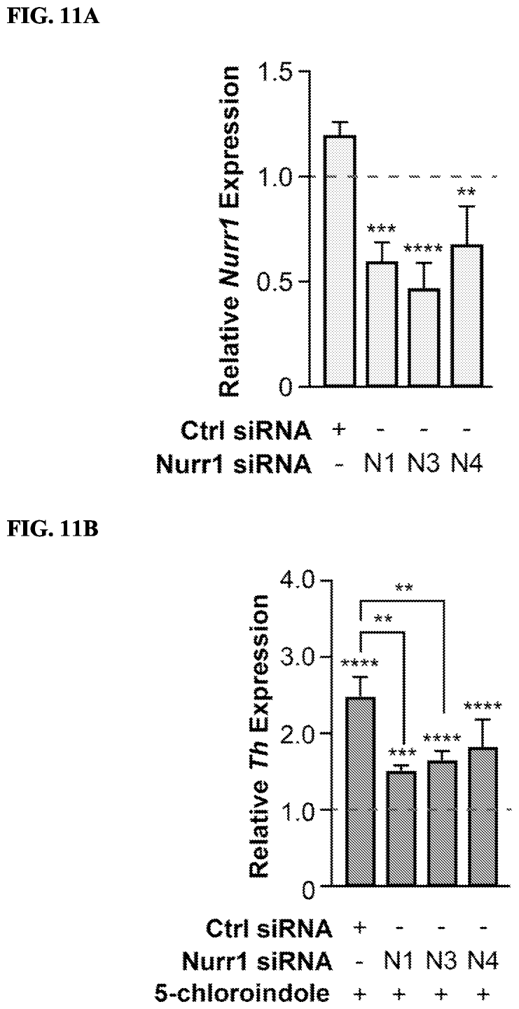

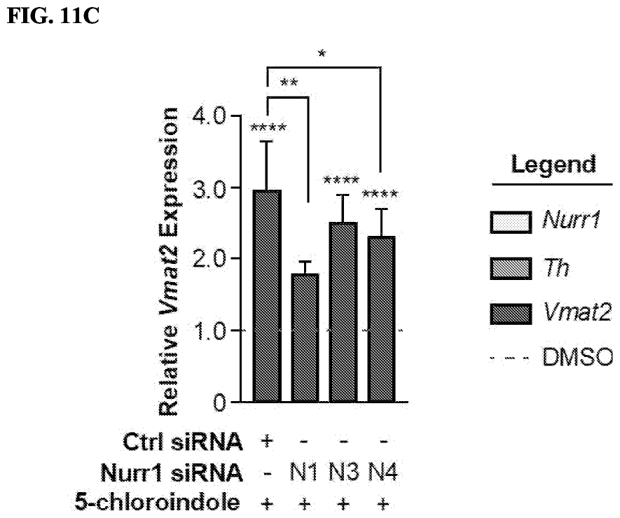

Control assays with 5-chloroindole demonstrate the observed binding affinity and effect on gene transcription is due to direct interaction of the small molecule with Nurr1 (FIGS. 9A-9B, FIGS. 10A-10B, FIGS. 11A-11C, and 12A-12D). First, increasing concentrations of the surfactant used in the MST binding assay only has a minor effect on the affinity of 5-chloroindole for the Nurr1 LBD (FIGS. 9A-9B), consistent with the observed effect on gene transcription being due to individual molecules binding specifically to the protein, as opposed to aggregated indoles driving the response through non-specific interactions. Second, 5-chloroindole stimulates the activity of Nurr1 in two different luciferase reporter assays, one relying a chimeric Nurr1-LBD_Gal4DBD protein binding to the Gal4 response element to drive luciferase expression, the other relying on binding of the full-length receptor to the NBRE response element (FIGS. 10A-10B). Third, the stimulatory effect of 5-chloroindole on the expression of dopamine-related target genes in MN9D cells is inhibited by siRNA specific for Nurr1 (FIGS. 11A-11C). Lastly, 5-chloroindole does not exhibit saturable binding to the LBD of RXRα (FIGS. 11A-11C), demonstrating the effect on transcription is not due to ligand binding to RXRα within an Nurr1-RXRα heterodimer.

To investigate the molecular basis for the intriguing difference in activity between the 5- versus 5,6-halogenated indoles, we mutated residues in the 566 site postulated to provide stabilizing interactions with these indoles. In our QM/MM models, these indoles are stabilized by interactions with Arg563 (cation-π) and His516 (halogen bond) (FIG. 4C), akin to the cation-π interaction and hydrogen bond observed in the DHI model (FIG. 3B). Using MST, we characterized the binding affinity of the four indoles for the Arg563 A la and His516Ala single and double mutant proteins, with close attention paid to changes in the response amplitude, a metric that is very sensitive to changes in the size, charge and solvation shell of the protein, and thus a reporter of differences in protein conformation.³⁶

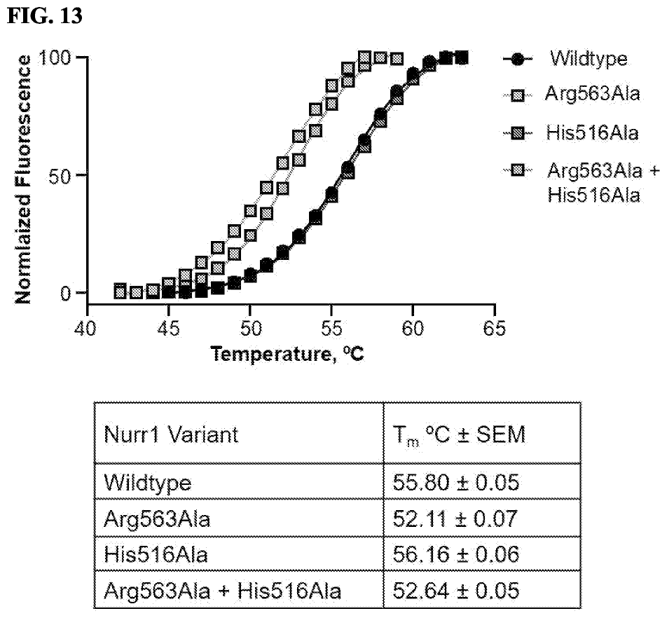

The binding of 5-bromoindole to Arg563 A la mutant Nurr1 LBD, alone or in combination with the His516Ala mutation, is completely eliminated, consistent with loss of a stabilizing cation-π interaction. The MST response amplitudes fall below the limit of robust signal detection for both mutants, and are significantly different from the large response amplitude observed for binding to the wildtype protein. Similarly, the response amplitude associated with 5-bromoindole binding to the His516Ala mutant is significantly different from that of the wildtype, though the binding affinity is statistically unchanged. The precise roles histidine side chains play in ligand-receptor interactions depend on the pKa of the side chain, which is influenced by the local environment inclusive of the ligand itself, making it generally difficult to predict the nature of the interaction.³⁷⁻⁴⁰ The affinity of 5-chloroindole for each of the mutants is improved (˜2-fold) relative to the wildtype protein, whereas the response amplitudes are markedly different. In striking contrast, the affinities of the corresponding dihalogenated indoles for each of the mutant proteins are reduced (˜3-7-fold), while the response amplitudes are not significantly different from that observed with the wildtype protein. Control assays suggest it is unlikely that changes in protein stability account for the observed differences in binding between the wildtype and mutant proteins. The Arg563 A la mutation reduces the protein stability by˜3.6°, likely because the guanidinium side chain forms a hydrogen bond with the carboxylate side chain of Glu445 in the unliganded structure (PDB:1OVL), and the His516Ala mutation slightly increases the thermal stability (˜0.4°) of the LBD (FIG. 13).

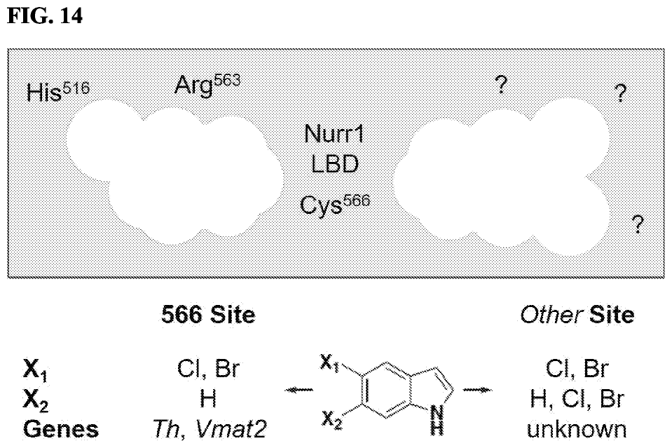

While the observed changes in binding affinity are equivocal, the changes in MST response amplitude between the wildtype and mutant proteins, combined with the effects of the 5- versus 5,6-substituted indoles on target gene transcription, point to a model in which there are two (or more) indole binding sites within the Nurr1 LBD (FIG. 14). Binding of 5-chloro and 5-bromoindole to the 566 site supports the transcription of target genes Th and Vmat2, whereas binding of 5,6-dichloro and 5,6-dibromoindole to the other site(s) does not. The notion that there are two (or more) indole binding sites within the Nurr1 LBD is in agreement with our previous study detailing the binding of DHI to Nurr1.²⁵ As well, computational studies surrounding the interaction of bis-indole compounds with Nurr1 predict two distinct binding sites for indoles within the LBD.⁴² Finally, biophysical and computational studies surrounding other small molecules targeting Nurr1 point to the presence of additional binding sites within the LBD.^{24, 42} Alternatively, it is possible these indoles bind to the same site, with the 5-substituted indoles inducing markedly different changes in the protein structure (and potentially interactions with co-regulatory proteins associated with transcription) than the corresponding 5,6-disubstituted indoles.

In conclusion, we have demonstrated that 5-chloroindole, a non-cytotoxic stable analog of the dopamine metabolite DHI, is suitable for directly probing the structure and function of Nurr1. The affinity of 5-chloroindole is comparable to DHI in vitro, whereas the potency in MN9D cells with respect to the expression of Th and Vmat2 is superior to that of DHI.

### REFERENCES FOR EXAMPLE 2

1. Decressac, M., Volakakis, N., Bjorklund, A., and Perlmann, T. (2013) Nat. Rev. Neurol. 9, 629-636. 2. Jakaria, M., Haque, M. E., Cho, D. Y., Azam, S., Kim, I. S., and Choi, D. K. (2019) Mol. Neurobiol. 56, 5799-5814. 3. Johnson, M. M., Michelhaugh, S. K., Bouhamdan, M., Schmidt, C. J., and Bannon, M. J. (2011) Front. Neurosci. 5, 135. 4. Jiang, C., Wan, X., He, Y., Pan, T., Jankovic, J., and Le, W. (2005) Exp. Neurol. 191, 154-162. 5. Kadkhodaei, B., Ito, T., Joodmardi, E., Mattsson, B., Rouillard, C., Carta, M., Muramatsu, S., Sumi-Ichinose, C., Nomura, T., Metzger, D., Chambon, P., Lindqvist, E., Larsson, N. G., Olson, L., Bjorklund, A., Ichinose, H., and Perlmann, T. (2009) J. Neurosci. 29, 15923-15932. 6. Kadkhodaei, B., Alvarsson, A., Schintu, N., Ramskold, D., Volakakis, N., Joodmardi, E., Yoshitake, T., Kehr, J., Decressac, M., Bjorklund, A., Sandberg, R., Svenningsson, P., and Perlmann, T. (2013) Proc. Natd. Acad. Sci. U.S.A 110, 2360-2365. 7. Zhang, L., Le, W., Xie, W., and Dani, J. A. (2012) Neurobiol. Aging 33, 100.1 e1007-1016. 8. Chu, Y., Le, W., Kompoliti, K., Jankovic, J., Mufson, E. J., and Kordower, J. H. (2006) J. Comp. Neurol. 494, 495-514. 9. Le, W., Pan, T., Huang, M., Xu, P., Xie, W., Zhu, W., Zhang, X., Deng, H., and Jankovic, J. (2008) J. Neurol. Sci. 273, 29-33. 10. Grimes, D. A., Han, F., Panisset, M., Racacho, L., Xiao, F., Zou, R., Westaff, K., and Bulman, D. E. (2006) Mov. Disord. 21, 906-909. 11. Le, W. D., Xu, P., Jankovic, J., Jiang, H., Appel, S. H., Smith, R. G., and Vassilatis, D. K. (2003) Nat. Genet. 33, 85-89. 12. Campos-Melo, D., Galleguillos, D., Sanchez, N., Gysling, K., and Andres, M. E. (2013) Front. Mol. Neurosci. 6, 44. 13. Valsecchi, V., Boido, M., Montarolo, F., Guglielmotto, M., Perga, S., Martire, S., Cutrupi, S., Iannello, A., Gionchiglia, N., Signorino, E., Calvo, A., Fuda, G., Chio, A., Bertolotto, A., and Vercelli, A. (2020) Dis. Model. Mech. 13. 14. Oswald, F., Kloble, P., Ruland, A., Rosenkranz, D., Hinz, B., Butter, F., Ramljak, S., Zechner, U., and Herlyn, H. (2017) Front. Cell. Neurosci. 11, 212. 15. Montarolo, F., Martire, S., Perga, S., and Bertolotto, A. (2019) nt. J. Mol. Sci. 20. 16. Dong, J., Li, S., Mo, J. L., Cai, H. B., and Le, W. D. (2016) CNS Neurosci. Ther. 22, 351-359. 17. Kim, C. H., Han, B. S., Moon, J., Kim, D. J., Shin, J., Rajan, S., Nguyen, Q. T., Sohn, M., Kim, W. G., Han, M., Jeong, I., Kim, K. S., Lee, E. H., Tu, Y., Naffin-Olivos, J. L., Park, C. H., Ringe, D., Yoon, H. S., Petsko, G. A., and Kim, K. S. (2015) Proc. Natl. Acad. Sci. U.S.A 112, 8756-8761. 18. McFarland, K., Spalding, T. A., Hubbard, D., Ma, J. N., Olsson, R., and Burstein, E. S. (2013) ACS Chem. Neurosci. 4, 1430-1438. 19. Smith, G. A., Rocha, E. M., Rooney, T., Barneoud, P., McLean, J. R., Beagan, J., Osborn, T., Coimbra, M., Luo, Y., Hallett, P. J., and Isacson, O. (2015) PLoS One 10, e0121072. 20. Zhang, Z., Li, X., Xie, W. J., Tuo, H., Hintermann, S., Jankovic, J., and Le, W. (2012) CNS Neurol. Disord. Drug Targets 11, 768-773. 21. De Miranda, B. R., Popichak, K. A., Hammond, S. L., Miller, J. A., Safe, S., and Tjalkens, R. B. (2015) Toxicol. Sci. 143, 360-373. 22. Hammond, S. L., Popichak, K. A., Li, X., Hunt, L. G., Richman, E. H., Damale, P. U., Chong, E. K. P., Backos, D. S., Safe, S., and Tjalkens, R. B. (2018) J. Pharmacol. Exp. Ther. 365, 636-651. 23. Munoz-Tello, P., Lin, H., Khan, P., de Vera, I. M. S., Kamenecka, T. M., and Kojetin, D. J. (2020) J. Med. Chem. 63, 15639-15654. 24. Willems, S., Ohrndorf, J., Kilu, W., Heering, J., and Merk, D. (2021) J. Med. Chem. 64, 2659-2668. 25. Bruning, J. M., Wang, Y., Oltrabella, F., Tian, B., Kholodar, S. A., Liu, H., Bhattacharya, P., Guo, S., Holton, J. M., Fletterick, R. J., Jacobson, M. P., and England, P. M. (2019) Cell Chem Biol 26, 674-685. 26. Rajan, S., Jang, Y., Kim, C. H., Kim, W., Toh, H. T., Jeon, J., Song, B., Serra, A., Lescar, J., Yoo, J. Y., Beldar, S., Ye, H., Kang, C., Liu, X. W., Feitosa, M., Kim, Y., Hwang, D., Goh, G., Lim, K. L., Park, H. M., Lee, C. H., Oh, S. F., Petsko, G. A., Yoon, H. S., and Kim, K. S. (2020) Nat. Chem. Biol. 16, 876-886. 27. Zucca, F. A., Segura-Aguilar, J., Ferrari, E., Munoz, P., Paris, I., Sulzer, D., Sarna, T., Casella, L., and Zecca, L. (2017) Prog. Neurobiol. 155, 96-119. 28. d'Ischia, M., Napolitano, A., and Pezzella, A. (2011) Eur. J. Org. Chem., 5501-5516. 29. Pezzella, A., Iadonisi, A., Valerio, S., Panzella, L., Napolitano, A., Adinolfi, M., and d'Ischia, M. (2009) J. Am. Chem. Soc. 131, 15270-15275. 30. Wang, Z., Benoit, G., Liu, J., Prasad, S., Aarnisalo, P., Liu, X., Xu, H., Walker, N. P., and Perlmann, T. (2003) Nature 423, 555-560. 31. Zhong, W., Gallivan, J. P., Zhang, Y., Li, L., Lester, H. A., and Dougherty, D. A. (1998) Proc. Natl. Acad. Sci. U.S.A 95, 12088-12093. 32. Davis, M. R., and Dougherty, D. A. (2015) Phys. Chem. Chem. Phys. 17, 29262-29270. 33. Waltman, R. J., Diaz, A. F., and Bargon, J. (1984) J. Phys. Chem. 88, 4343-4346. 34. Napolitano, A., d'Ischia, M., and Prota, G. (1988) Tetrahedron 44, 7265-7270. 35. Jennings, P., Jones, A. C., Mount, A. R., and Thomson, A. D. (1997) Journal of the Chemical Society, Faraday Transactions 93, 3791-3797. 36. Jerabek-Willemsen, M., Wienken, C. J., Braun, D., Baaske, P., and Duhr, S. (2011) Assay Drug Dev Technol 9, 342-353. 37. Lin, F. Y., and MacKerell, A. D., Jr. (2017) J. Phys. Chem. B 121, 6813-6821. 38. Wilcken, R., Zimmermann, M. O., Lange, A., Joerger, A. C., and Boeckler, F. M. (2013) J. Med. Chem. 56, 1363-1388. 39. Hansen, A. L., and Kay, L. E. (2014) Proc. Natl. Acad. Sci. U.S.A 111, E1705-1712. 40. Politzer, P., Murray, J. S., and Clark, T. (2013) Phys. Chem. Chem. Phys. 15, 11178-11189. 41. Li, X., Tjalkens, R. B., Shrestha, R., and Safe, S. (2019) Chem. Biol. Drug Des. 94, 1711-1720. 42. de Vera, I. M., Giri, P. K., Munoz-Tello, P., Brust, R., Fuhrmann, J., Matta-Camacho, E., Shang, J., Campbell, S., Wilson, H. D., Granados, J., Gardner, W. J., Jr., Creamer, T. P., Solt, L. A., and Kojetin, D. J. (2016) ACS Chem. Biol. 11, 1795-1799.

### Example 3: Additional Data and Experimental Procedures

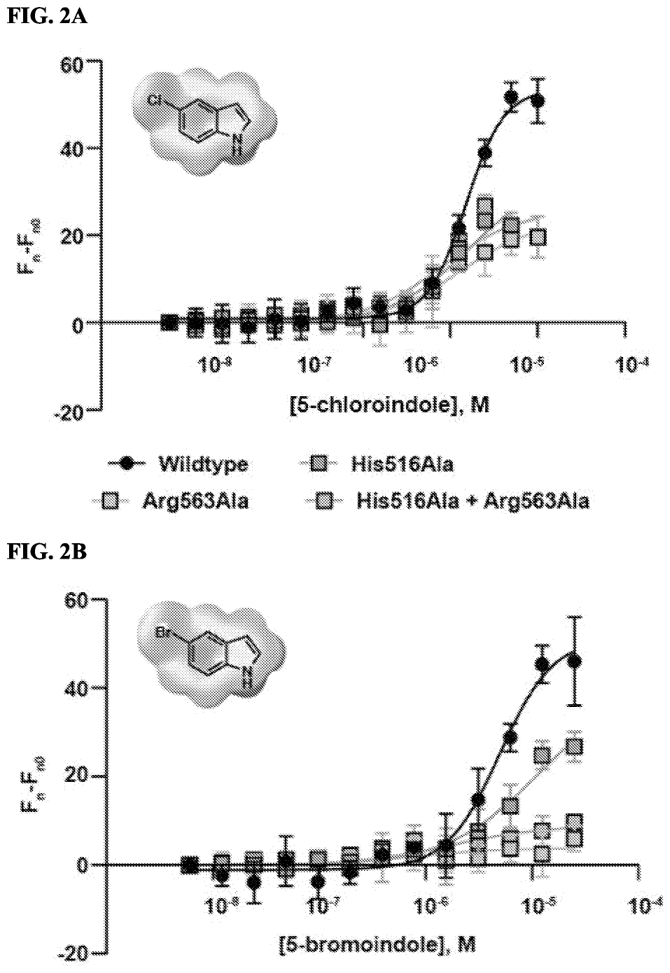

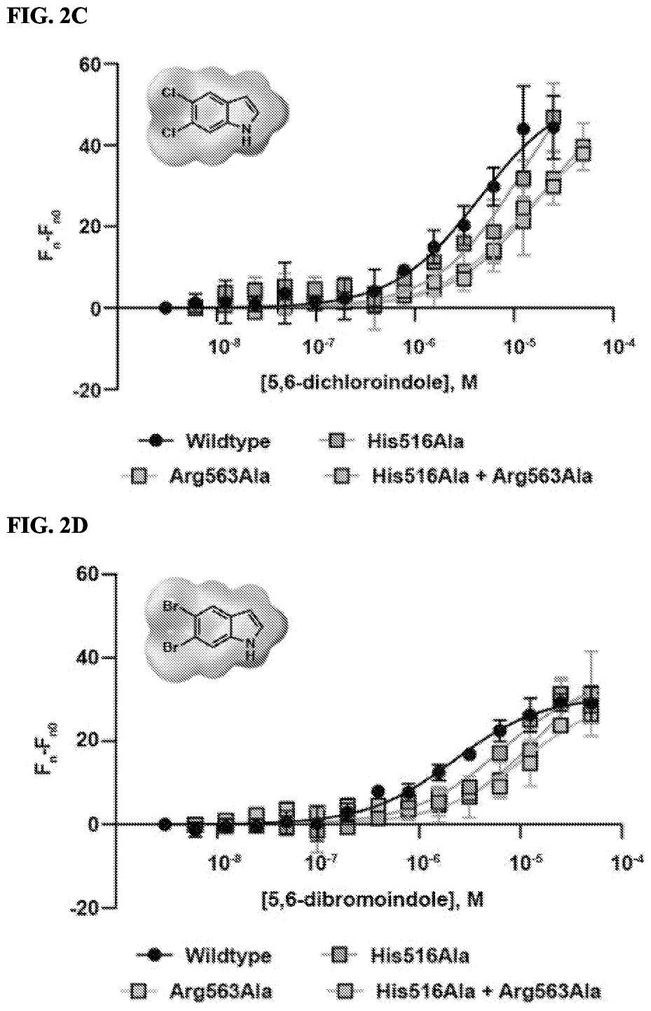

TABLE 2
Summary of the binding data values (K_{D}, MST amplitude) for the graphs
shown in FIGS. 2A-2D. All experimental values are the result of three or more independent
biological replicates ± standard deviation.

| | | Affinity | Response |
|---|---|---|---|
| Compound | Nurr1 Variant | (K_{D}, μM) | Amplitude |
| 5-bromoindole | Wildtype | 5.0 + 0.7 | 50 + 4 |
| | Arg563Ala | ND | ≤10 (****) |
| | His516Ala | 7.3 + 2.1 | 33 + 5 (*) |
| | Arg563Ala + His516Ala | ND | ≤10 (****) |
| 5-chloroindole | Wildtype | 15.0 + 1.2 | 54 + 2 |
| | Arg563Ala | 8.3 + 1.7 | 19 + 2 (****) |
| | His516Ala | 8.6 + 1.2 | 25 + 2 (****) |
| | Arg563Ala + His516Ala | 7.3 + 0.8 | 23 + 1 (****) |
| 5,6-dibromoindole | Wildtype | 2.2 + 0.2 | 31 + 1 |
| | Arg563Ala | 11.0 + 2.8 | 32 + 3 (ns) |
| | His516Ala | 6.4 + 1.3 | 35 + 2 (ns) |
| | Arg563Ala + His516Ala | 15.6 + 4.3 | 43 + 5 (ns) |
| 5,6-dichloroindole | Wildtype | 4.5 + 0.9 | 54 + 4 |
| | Arg563Ala | 13.1 + 2.2 | 47 + 3 (ns) |
| | His516Ala | 12.0 + 3.6 | 67 + 9 (ns) |
| | Arg563Ala + His516Ala | 17.9 + 3.8 | 54 + 5 (ns) |

Chemicals and Reagents

The indoles used in the present study were purchased from Ambeed or Fisher Scientific. All other chemicals were purchased from Millipore, Sigma, or ThermoFisher Scientific, unless otherwise indicated. The pYFJ16-LplA(W37V) plasmid used to express the coumarin ligase (LpIA) was purchased from Addgene. The MN9D Tet-ON cell line was graciously provided by Dr. Thomas Perlmann (Karolinska Institute). The reporter plasmid NBREx3-POMC-Luc was graciously provided by Dr. Jacques Drouin (Institut de Recherches Cliniques de Montréal, Canada).

Computational Methods

The molecular electrostatic potential surface for each indole was calculated using the 6-31G** basis sets and the B3LYP-D3 functional in water (PBS solvent model), and bromine atoms were treated with the LAV2P**. All calculations were performed using Jaguar (Schrodinger®) software.

Models for non-covalent binding of substituted-indoles to Nurr1 within the DHI-binding 566 site were prepared according to quantum mechanics-molecular mechanics (QM/MM) calculations using the dispersion-corrected (D3) Density Functional Theory (DFT) and the LACVP* basis sets for the QM region, and the MM OPLS2005 force field for the other residues. Qsite (Schrodinger®) was used for these calculations.

Single point interaction energies between the side chain of His516 and the C₅-substituted indoles were calculated using Jaguar (Schrodinger®) with the LMP2/cc-pVDZ** level of theory. Energies were calculated for the gas phase, rather than with implicit solvation models, as nuclear receptor ligand binding pockets are traditionally hydrophobic cavities; this is certainly the case for the previously identified DHI-binding 566 site (PDB:1OVL). The coordinates of the complexes used for these calculations were taken from QM/MM optimized, non-covalently bound indole-Nurr1 structures at the DFT-D3/LACVP* level of theory. All energy values were calculated in kcal mol¹.

The pKa predictions for His516 were made using by propKa 3.1 after QM/MM optimization of the non-covalent ligand-bound Nurr1 structures. In the starting structure (PDB ID: 6D DA), the predicted pKa for His516 is 6.5. Upon ligand binding and optimization, the pKa value of His516 is increased, especially for substituted-indoles containing hydrogen bond acceptors at C-5 position.

DNA Constructs

The plasmid used for the expression of LAP2-tagged Nurr1 LBDs (LAP2 Nurr1) was prepared by GenScript (Piscataway, N.J.) as the product of gene synthesis and subcloning into pET-21a(+) Vector (GenScript) using the Ndel and Xhol sites within the MCS. The protein sequence of the resulting protein is shown in Table 3. Mutants of LAP2 Nurr1 were prepared by GenScript starting from LAP2 Nurr1 vector.

TABLE 3

| Construct | |
|---|---|
| Name | Sequence |
| LAP2 Nurr1 | MKKGHHHHHHGFEIDKVWYDLDAGAISLISAL |
| | VRAHVDSNPAMTSLDYSRFQANPDYQMSGDDT |
| | QHIQQFYDLLTGSMEIIRGWAEKIPGFADLPK |
| | ADQDLLFESAFLELFVLRLAYRSNPVEGKLIF |
| | CNGVVLHRLQCVRGFGEWIDSIVEFSSNLQNM |
| | NIDISAFSCIAALAMVTERHGLKEPKRVEELQ |
| | NKIVNCLKDHVTFNNGGLNRPNYLSKLLGKLP |
| | ELRTLCTQGLQRIFYLKLEDLVPPPAIIDKLF |
| | LDTLPF (SEQ ID NO: 7) |
| LAP2 RXRα | MKKGHHHHHHGSGSENLYFQSGSGSGFEIDKV |
| | WYDLDAGSGSDMPVERILEAELAVEPKTETYV |
| | EANMGLNPSSPNDPVTNICQAADKQLFTLVEW |
| | AKRIPHFSELPLDDQVILLRAGWNELLIASFS |
| | HRSIAVKDGILLATGLHVHRNSAHSAGVGAIF |
| | DRVLTELVSKMRDMQMDKTELGCLRAIVLFNP |
| | DSKGLSNPAEVEALREKVYASLEAYCKHKYPE |
| | QPGRFAKLLLRLPALRSIGLKCLEHLFFFKLI |
| | GDTPIDTFLMEMLEAPHQMT |
| | (SEQ ID NO: 8) |

Synthesis and Purification of the Azide-Reactive Fluorescein Probe for MST

The dibenzocyclooctyne (DBCO)-5/6-carboxyfluorescein probe was synthesized according to previously reported procedures (Patent US20150125904A1). Briefly, dibenzocyclooctyne-amine (3.2 mg, 11.5 μmol) in 540 μL anhydrous DMF was added to 5/6-carboxyfluorescein N-succinimidyl ester (6 mg, 12.7 μmol) and triethylamine (4.9 μL, 35.7 μmol). After stirring overnight at ambient temperature, the solvent was removed by lyophilization and resulting oil was resuspended in EtOAc and extracted against 1 M HCl, followed by saturated NaCl. The organic layer was dried over anhydrous MgSO₄ and then concentrated to dryness via rotary evaporation to give the crude alkyne. The desired product was purified to homogeneity using preparative thin layer chromatography (EtOAc), eluted from the silica gel (EtOAc:MeOH, 95:5), and then concentrated to dryness to give the final product, dibenzocyclooctyne (DBCO)-5/6-carboxyfluorescein, in an overall yield of 60%. ESI-MS characterization [M+H]⁺ gave 635.7 observed; 635.2 calculated.

Protein Expression and Purification

The Nurr1 LBD protein, containing the N-terminal “LAP2” sequence that is recognized by a “coumarin ligase”, was expressed and purified using metal affinity and size-exclusion chromatography according to the previously reported protocol, except that the TEV cleavage step and reverse metal affinity chromatography were omitted.¹ The RXRα LBD protein, containing the N-terminal “LAP2” sequence, was expressed and purified identically to the Nurr1 LBD protein with minor modifications. Specifically, the elution of protein from Talon resin was performed by a step gradient of 10 CV each of 50 mM, 100 mM, 200 mM and 300 mM imidazole in 50 mM Tris-HCl buffer containing 300 mM NaCl at pH 7.8. Purity of protein in each fraction was analyzed by SDS PAGE and fractions eluted with 100-300 mM imidazole were pooled and concentrated. Resulting protein solution was then applied to a S75 10/300 SEC column (GE Healthcare Life Sciences) using a running buffer composed of 50 mM Tris-HCl, 100 mM KCl, 1 mM DTT, 10% glycerol at pH 8.0. The sequence of the construct (prepared by GenScript) is shown above in Table 3.

Labeling Protein with Fluorescein for MST Assays

The fluorescein probe was ligated to the N-terminal LAP2 tag within Nurr1 LBD and RXR LBD using a re-engineered version of the enzyme lipoic acid ligase (LpIA) from Escherichia coli as previously reported.^{2, 3} Briefly, the plasmid harboring the gene coding for “coumarin ligase” [pYFJ16-LplA(W37V); Addgene] was transformed into BL21(DE3) cells (New England BioLabs) and a single colony was subsequently used to inoculate LB media supplemented with 100 μg/mL ampicillin, and the culture was grown at 37° C. until reaching an OD₆₀₀ of 0.9, at which point protein expression was induced by adding IPTG (100 μM final concentration) and incubation continued for 16 hours at 25° C. Next, the cells were harvested by centrifugation (3,500 g, 20 minutes, 4° C.) and the pellet was resuspended in lysis buffer (50 mM Tris base, 300 mM NaCl, pH 7.8) containing cOmplete mini EDTA-free protease inhibitor cocktail (Roche). Cells were lysed by continuous passage at 15,000 psi using C3 Emulsiflex (Avestin). The extract was cleared by centrifugation (21,000 g, 45 minutes, 4° C.) and the His₆-tagged enzyme was purified using Ni-NTA agarose (Qiagen). Fractions were analyzed by 12% SDS-PAGE followed by Coomassie staining. Fractions containing LplA were pooled and dialyzed against 20 mM HEPES, 150 mM NaCl, 1 mM DTT, 10% glycerol, pH 8.0. The protein concentration was determined by measuring the A280 and using the calculated extinction coefficient 41,940 M⁻¹ cm⁻¹.

Sequence specific (LAP2-tag) incorporation of the fluorescein probe was achieved according to the previously reported protocol.⁴ A typical reaction contained LAP2-tagged protein (20 μM), buffer (25 mM sodium phosphate, pH 7.0, 2 mM magnesium acetate, 1 mM ATP), 10-azadecanoic acid (100 μM), and the enzyme W37V LplA (1 μM). After incubating at 30° C. for 1 h, the reaction was supplemented with 200 μM DBCO-linked 5(6)-carboxyfluorescein probe and allowed to incubate at ambient temperature for 30 min before being buffer exchanged into 25 mM HEPES, pH 7.4, 150 mM NaCl. The concentration of fluorescent label was determined by UV-vis spectroscopy using the extinction coefficient ε₄₉₃=70,000 M⁻¹ cm⁻¹ for fluorescein.

Microscale Thermophoresis Assay

Concentration-dependent association of the indoles with the Nurr1 LBD was carried out using microscale thermophoresis. Stock solutions (10 mM in DMSO) of each indole were serially diluted (200 μM indole, 0.5× dilutions down to 0.0061 μM) in MST buffer (25 mM HEPES, pH 7.4, 150 mM NaCl, and 0.1% Pluronic F127) containing 2% DMSO. The dilutions were carried out with 4% DMSO in the MST buffer for a total of 16 concentrations. Equivalent volumes and concentrations of the fluorescently labeled Nurr1 LBD in MST buffer were added to each ligand dilution in the series to reach a final concentration of 75 nM labeled protein. After incubating for 20 minutes, the samples were loaded into Monolith NT.115 Capillaries (Nanotemper).

Data were collected using the Monolith NT.115 System (Nanotemper), with settings for all samples at 40% excitation power and 40% MST power. The initial fluorescence was recorded for 3 sec and the thermophoresis fluorescence response was recorded for 20 sec. The data was inspected with Palmist software⁵, and data points affected by initial fluorescence quenching or photobleaching were eliminated. The fluorescent response for each sample was normalized to the initial fluorescence using Palmist software to provide the values of thermophoresis (Fₙ). The resulting data was used to generate a plot of the change in thermophoresis (Fₙ-Fₙ₀, where Fₙ=thermophoresis and Fₙ₀=thermophoresis response in unbound range) versus concentration of the ligand. GraphPad Prism v. 8.3.0 software was used to fit the resulting data to a mass action equation for a specific binding with Hill slope, solving for K_{D}: Fₙ-Fₙ₀═Fₘₐₓ·[L]ⁿ/(K_{D}ⁿ+[L]ⁿ), where Fₘₐₓ=maximum amplitude of thermophoresis, [L]=concentration of the ligand at a specific point, K_{D}=dissociation constant, n_{H}=Hill Coefficient.

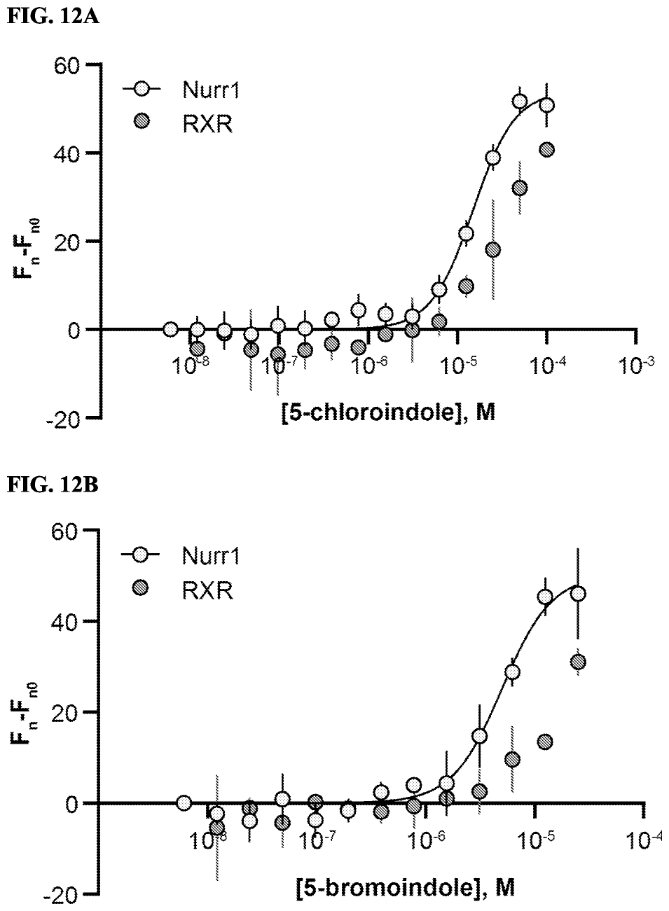

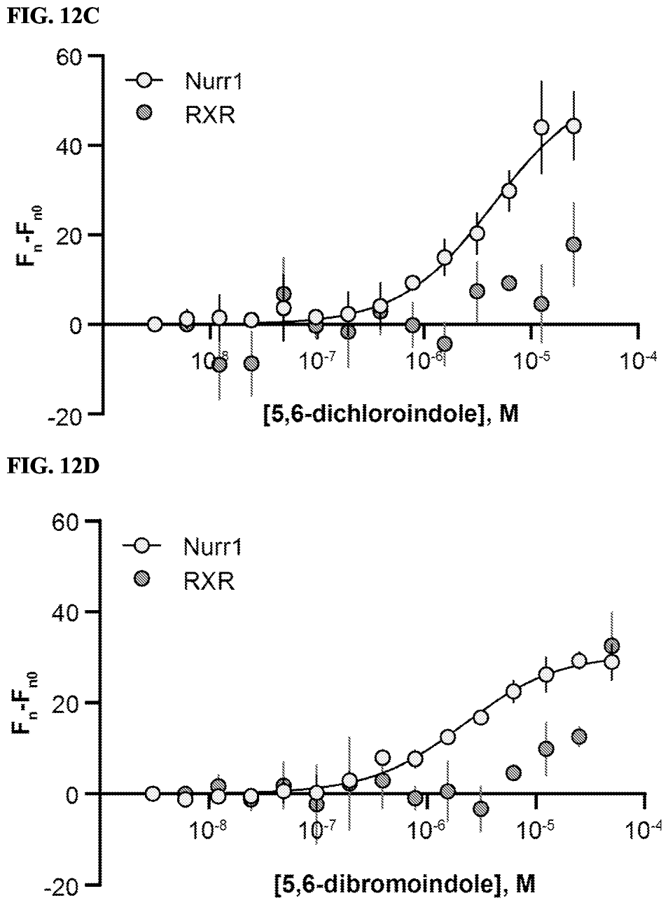

MST binding assays for the 5-chloroindole, 5-bromoindole, 5,6-dichloroindole, and 5,6-dibromoindole were also run using an unrelated protein, the RXRα LBD, and demonstrate that the signal changes observed for binding to the Nurr1 LBD are not dominated by non-specific binding artifacts (FIGS. 10A-10B). To investigate the potential impact of compound nano-aggregation on binding affinity, we used dynamic light scattering to inspect the aggregation of 5-chlorindole in solution and repeated the MST binding experiments in the presence of increasing concentrations of the surfactant Pluoronic F127 (FIGS. 12A-12D). The nano-aggregation properties of 5-chloroindole were found to be minimal, ruling out the possibility that the observed concentration-dependent changes in the MST signal are dominated by compound aggregation. As well, increasing concentrations of the surfactant Pluronic F127 had small effects (<2-fold) on the affinity of 5-chloroindole for the Nurr1 LBD (FIGS. 12A-12D). Furthermore, UV/VIS spectroscopy (A₂₈₀) of both 5-chloro- and 5-bromoindole, under conditions equivalent to those used in the MST binding assays (0.1% Pluronic F127), reveals that the absorbance of both compounds remains linear over all concentrations tested, indicating that these compounds do not precipitate under the assay conditions.

Differential Scanning Fluorimetry Assay

The Nurr1 LBD protein was buffer exchanged into 25 mM HEPES, 150 mM NaCl, pH 7.4 using a Zeba Spin Desalting Column (ThermoFisher). The DSF assays were carried out in a final volume of 30 μL, comprised of 4 mM protein, 1× SYPRO Orange (ThermoFisher/Life Technologies, from 5000× stock), and buffer comprised of 25 mM HEPES, pH 7.4, 150 mM NaCl. Samples were allowed to incubate in the dark for 30 min at 25° C., prior to exposure to thermal gradient. Fluorescence was monitored using the CFX Connect Real-Time PCR Detection System (BioRad) in a 96-well plate (BioRad). The thermal gradient was executed from 25° C. to 95° C. at a rate of 0.05° C./s. The fluorescence response was normalized so that 0% and 100% are defined as the smallest and the largest mean in each dataset, correspondingly. Melting temperatures, Tₘ (the inflection point of the sigmoidal curve), was calculated using the Boltzmann sigmoid equation: Y=bottom+(top-bottom)/(1+exp(Tₘ−x/slope)), where bottom and top are the values of the minimum and maximum intensities.

Dynamic Light Scattering Assay

5-Chloroindole was serially diluted from 10 mM DMSO stock into MST buffer supplemented with various amounts of Pluronic F127 (0.1%, 0.2%, 0.5%, 1.0%) at room temperature for a final concentration of 0.2% DMSO. Measurements were made using a DynaPro MS/X (Wyatt Technology) with a 55 mW laser at 826.6 nm, using a detector angle of 90°. The laser power was 100%, and the acquisition time was 2 s. Histograms represent the average of three independent data sets, each with at least 10 measurements.

Cellular Assays

Target Gene Transcription Assays. These assays were carried out using standard protocols. MN9D TET-ON frozen cell stocks (P5) were thawed and grown for 60-72 h on poly-D-lysine pre-treated culture dishes (100 mm) in Dulbecco's Modified Eagle Medium/Nutrient Mixture F-12 (DMEM/F-12; Gibco) supplemented with 5% Tet System Approved FBS (Takara Bio USA) at 37° C., 5% C₀₂ to ˜80% confluency. The resulting cells were then trypsinized with 0.25% Trypsin-EDTA (Gibko) and diluted to 2·10⁵ cells/ml with fresh medium. The resulting cell suspension (0.8 mL) was added to 2× concentrated compound in the same medium containing 0.2% DMSO (0.8 mL) in an Eppendorf tube. The cell suspension with compound or vehicle (DMSO) was then seeded onto a 24-well plate pre-treated with Poly-D-Lysine at 0.8 mL per well. Assays were performed under conditions of basal Nurr1 expression, without induction of additional Nurr1 expression using doxycycline.

After 24 h, total RNA was extracted the cells in each well using the Quick-RNA MiniPrep Plus Kit (Zymo Research), according to the manufacturer's instructions. The cDNA was then synthesized from 1000 ng of purified RNA using High-Capacity cDNA Reverse Transcription Kit (Applied Biosystems) and used as template. The qPCR as performed using iTaq Universal SYBR Green Supermix (BioRad) and CFX96 Real-Time Detection System machine (BioRad). Briefly, qPCR was performed in hard-shell 96-well PCR plates (BioRad) using cDNA corresponding to 8.75 ng of starting total RNA in a volume of 15 μl, containing 7.5 μl of SYBR Green Supermix, and 1 μL of 10 μM forward and reverse primers. Cycling parameters for qPCR included an initial denaturation at 95° C. for 3 min, followed by 40 cycles of 95° C. for 5 s and annealing at 56° C. for 30 s. The forward and reverse primers (Table 4) were ordered from IDT. Gene expression was quantified by the comparative2-ΔΔCt method, with the mouse housekeeping gene hypoxanthine-guanine phosphoribosyltransferase (Hprt) used as internal reference to determine the relative mRNA expression. Transcript levels for target genes were normalized to the housekeeping gene Hprt and fold change was compared to gene expression levels from vehicle (DMSO) only treated cells. GraphPad Prism 8 software was used for statistical analysis. Two-way ANOVA was applied for DMSO fold change vs compound. Results are from three independent experiments. Relative average expression±SD; *p<0.05, **p<0.01, ***p<0.001, ****p<0.0001 by ANOVA in comparison expression with 0 μM compound (DMSO only).

TABLE 4
Sequences of primers used in RT-qPCR

| Target | | Sequence | |
|---|---|---|---|
| Gene | Primer | (5′ to 3′) | Reference |
| Hprt | FW | TGGGAGGCCATCACA | Volpicelli, |
| | | TTGT | Floriana, et al. |
| | | (SEQ ID NO: 9) | PloS one 7.2 |
| | REV | AATCCAGCAGGTCAG | (2012):e30661. |
| | | CAAAGA | |
| | | (SEQ ID NO: 10) | |
| Nurr1 | FW | CAACTACAGCACAGG | |
| | | CTA | |
| | | (SEQ ID NO: 11) | |
| | REV | GCATCTGAATGTCTT | |
| | | CTACCTTAATG | |
| | | (SEQ ID NO: 12) | |
| Th | FW | TCCAACCTTTCCTGG | Hwang, Dong-Youn, |
| | | CCCAG | et al. Journal of |
| | | (SEQ ID NO: 13) | neurochemistry |
| | REV | GCATGAAGGGCAGGA | 111.5 (2009): |
| | | GGAAT | 1202-1212. |
| | | (SEQ ID NO: 14) | |
| Vmat2 | FW | GAAGTCCACCTGCTA | Designed in |
| | | AGGAAGAA | this work. |
| | | (SEQ ID NO: 15) | |
| | REV | TCACTGGAGACACAT | |
| | | GTACACAG | |
| | | (SEQ ID NO: 16) | |

Nurr1 knockdown (siRNA) assays were completed according to standard protocols. Briefly, MN9D cells were resuspended in DMEM/F-12 with 5% FBS at 1·10⁶ cells/mL, and reverse-transfected with either control or Nurr1 siRNAs (40 nM final concentration) by combining 4 mL of cell suspension with 1 mL of Opti-MEM containing 200 nM siRNA and 10 μL Lipofectamine 2000 (Invitrogen, cat #:11668019). The resulting cell suspension was plated on poly-D-lysine treated six-well plates at 2.5 mL/well, and allowed to incubate for 24 h at 37° C., in 5% CO₂ incubator. After 24 hours, the cells were trypsinized, resuspended in DMEM/F-12 with 5% FBS at 2·10⁵ cells/mL, mixed with equal volume of 20 μM 5-chloroindole in the same media containing 0.2% DMSO (prepared by diluting DMSO stock of 5-chloroindole (10 mM) in warm DMEM/F-12 with 5% FBS; for the DMSO control an equivalent volume of DMSO was used instead of the compound stock), and immediately re-plated on poly-D-lysine pre-treated 24-well plates at 1-10⁵ cells/well and incubated as above. After 24 hours, total RNA was isolated and the target gene transcription assay (qPCR) was performed as described above. Nurr1 siRNA N1 were purchased from Sigma (Cat No. SASI_Mm02_00322368), and the sequences were as follows: 5′-GAA UCA GCU UUC UUA GAA U[dT][dT]-3′ (SEQ ID NO:17) (sense) and 5′-AUU CUA AGA AAG CUG AUU C[dT][dT]-3′ (SEQ ID NO:18) (antisense). Nurr1 siRNA N3 and N4 were ordered from IDT, and the sequences were as follows: 5′-GCAUCGCAGUUGCUUGACATT (SEQ ID NO:19) (N3 sense) and 5′-UGUCAAGCAACUGCGAUGCGT (SEQ ID NO:20) (N3 antisense); 5′-CUAGGUUGAAGAUGUUAUAGGCACT (SEQ ID NO:21) (N4 sense) and 5′ AGUGCCUAUAACAUCUUCAACCUAGAA (SEQ ID NO:22) (N4 antisense).⁶

GFP negative control DsiRNA were purchased from IDT (Cat No. 51-01-05-06).

Cytotoxicity assays were carried out using the CytoTox-Glo Cytotoxicity Assay Kit (Promega) according to the manufacturer's instructions. Briefly, MN9D cells were resuspended in DMEM/F-12 with 5% FBS at 2·10⁵ cells/ml and added to an equal volume of two-fold concentrated compound in the same media containing 0.2% DMSO (prepared by diluting DMSO stock of the compound in warm DMEM/F-12 with 5% FBS; for DMSO control equivalent volume of DMSO was used instead of the compound stock), and immediately re-plated onto poly-D-lysine pre-treated 96-well white-walled flat clear bottom plate (Corning cat #3903) at a density of 1·10⁴ cells/well (100 μL/well), 3 replicas per compound. As a background (BG) control, for each of the compound (or DMSO) equivalent volume of compound mixed with DMEM/F-12+5% FBS was plated. After 24 h treatment at 37° C., in 5% C₀₂ incubator, 50 μL of CytoTox-Glo™ Cytotoxicity Assay Reagent was added to each well, mixed briefly by orbital shaking and incubated for 15 min at ambient temperature. Luminescence signal corresponding to dead cells was measured using Biotek Synergy H4 hybrid microplate reader. After the measurement, 50 μL of Lysis Reagent was added to each well, mixed, incubated for 15 min at ambient temperature, and total luminescence was measured. The percentage of viable cells was then calculated as follows: Percent Viable Cells (%)=100% (Total cell luminescence (test compound)—Dead cell luminescence (test compound))/(Total cell luminescence (DMSO)—Dead cell luminescence (DMSO)).

Luciferase Reporter Assays. These assays were executed using standard protocols. MN9D TET-ON cells (P5) were grown for 60-72 h (see above) before being seeded at 1-10⁵ cell/well in 24-well plate in DMEM/F12+5% TET-ON approved FBS 3 h before transfection. Cells were transfected using Lipofectamine 2000 (Invitrogen) and plasmid DNAs according to manufacturer's instructions. Lipofectamine/DNA complexes were prepared in Opti-Mem medium (Gibco) and incubated with cells overnight. The Nurr1-LBD_Gal4-DBD expressing plasmid was prepared by subcloning the Nurr1 LBD fragment into μM plasmid (Clontech) containing GAL4 DBD. The reporter plasmid pGL4.35 (luc2P/9XGAL4 UAS/Hygro) Vector (Promega) contains nine repeats of GAL4 UAS (Upstream Activator Sequence) and drives transcription of the luciferase reporter gene luc2P in response to binding of Nurr-LBD_Gal4-DBD chimeric protein. The pRL-null (Promega) plasmid expressing renilla luciferase was used as an internal control. The amounts of pM-Nurr1-LBD_Gal4-DBD, pGL4.35, pRL-null and Lipofectamine 2000 used for transfections were 100 ng, 100 ng, 200 ng and 1 μL per well respectively. Alternatively, cells were co-transfected with the reporter plasmid NBREx3-POMC-Luc containing three copies of the NBRE sequence (5′-GATCCTCGTGCGAAAAGGTCAAGCGCTA-3′ (SEQ ID NO:23)) subcloned into the pXP1-luc plasmid containing the minimal (positions −34 to +63) POMC promoter as described previously⁷, and the pRL-null plasmid. The amounts of NBREx3-POMC-Luc, pRL-null and Lipofectamine 2000 used for transfecting cells were 100 ng, 200 ng and 1 μL per well respectively.

Transfected cells were treated with increasing concentrations of 5-chloroindole or vehicle only for 6 h, after which time the media was aspirated and luciferase activity was measured. Cells from each well were incubated for 15 min with 220 μL/well of Dual-Glo® Luciferase Reagent (Promega) at room temperature upon rotation on the orbital shaker. Resulting lysates were cleared from the cell debris by centrifugation for 2 min at 16,000 rcf. Resulting solutions were transferred to a white opaque 96-well plate (65 μL/well; 3 wells/sample) and firefly luciferase activity was measured using Veritas Microplate Luminometer (Turner BioSystems, Sunnyvale, Calif.). An equal volume of Dual-Glo® Stop & Glo® Reagent (Promega) was added to each well. Renilla luciferase activity was measured after 20 min of incubation of the plate inside the luminometer. Experimental values are expressed as the average of firefly/renilla luciferase activity±standard deviation (for three independent biological replicates). One-way Analysis of Variance (ANOVA) was used to determine statistical significance using GraphPad Prism 8.3.0 software, with *p<0.05, **p<0.01, ***p<0.001, ****p<0.0001 in comparison with 0 μM compound (DMSO only).

### References for Example 3

1. Bruning, J. M., Wang, Y., Oltrabella, F., Tian, B., Kholodar, S. A., Liu, H., Bhattacharya, P., Guo, S., Holton, J. M., Fletterick, R. J., Jacobson, M. P., and England, P. M. (2019) Cell Chem Biol 26, 674-685. 2. Uttamapinant, C., White, K. A., Baruah, H., Thompson, S., Fernandez-Suarez, M., Puthenveetil, S., and Ting, A. Y. (2010) Proc. Natl. Acad. Sci. U.S.A 107, 10914-10919. 3. Fernandez-Suarez, M., Baruah, H., Martinez-Hernandez, L., Xie, K. T., Baskin, J. M., Bertozzi, C. R., and Ting, A. Y. (2007) Nat. Biotechnol. 25, 1483-1487. 4. Yao, J. Z., Uttamapinant, C., Poloukhtine, A., Baskin, J. M., Codelli, J. A., Sletten, E. M., Bertozzi, C. R., Popik, V. V., and Ting, A. Y. (2012) J. Am. Chem. Soc. 134, 3720-3728. 5. Scheuermann, T. H., Padrick, S. B., Gardner, K. H., and Brautigam, C. A. (2016) Anal. Biochem. 496, 79-93. 6. De Miranda, B. R., Popichak, K. A., Hammond, S. L., Jorgensen, B. A., Phillips, A. T., Safe, S., and Tjalkens, R. B. (2015) Mol. Pharmacol. 87, 1021-1034. 7. Maira, M., Martens, C., Batsche, E., Gauthier, Y., and Drouin, J. (2003) Mol. Cell. Biol. 23, 763-776.

1. A method of treating Parkinson's disease in a subject in need thereof, the method comprising administering to the subject in need thereof a therapeutically effective amount of a compound, or a pharmaceutically acceptable salt thereof, wherein the compound has the formula 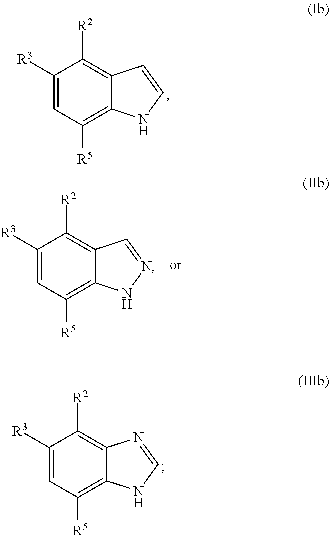
wherein
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —SC(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, —SF₅, —SSR^{2D}, —SiR^{2A}R^{2B}R^{2C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is —Br or —Cl;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —SC(O)R^{5C}, —C(O)OR^{5C}, —C(O)NR^{5A}R^{5B}, —OR^{5D}, —SR^{5D}, —SeR^{5D}, —NR^{5A}SO₂R^{5D}, —NR^{5A}C(O)R^{5C}, —NR^{5A}C(O)OR^{5C}, —NR^{5A}OR^{5C}, —N₃, —SF₅, —SSR^{5D}, —SiR^{5A}R^{5B}R^{5C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{5A}, R^{5B}, R^{5C}, and R^{5D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl; R^{5A} and R^{5B} substituents bonded to the same nitrogen atom may be joined to form a substituted or unsubstituted heterocycloalkyl or substituted or unsubstituted heteroaryl;
n2 and n5 are independently an integer from 0 to 4;
m2, m5, v2, and v5 are independently 1 or 2; and
X² and X⁵ are independently —F, —Cl, —Br, or —I.

2. A compound having the formula 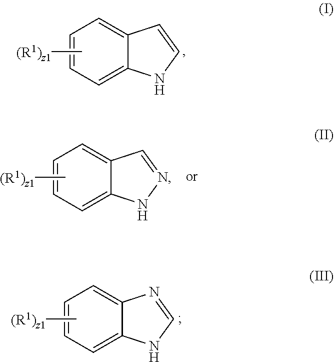
wherein
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —SC(O)R^{1C}, —C(O)OR^{1C}, —C(O)NR^{1A}R^{1B}, —OR^{1D}, —SR^{1D}, —SeR^{1D}, —NR^{1A}SO₂R^{1D}, —NR^{1A}C(O)R^{1C}, —NR^{1A}C(O)OR^{1C}, —NR^{1A}OR^{1C}, —N₃, —SF₅, —SSR^{1D}, —SiR^{1A}R^{1B}R^{1C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and RB substituents bonded to the same nitrogen atom may be joined to form a substituted or unsubstituted heterocycloalkyl or substituted or unsubstituted heteroaryl;
n1 is independently an integer from 0 to 4;
m1 and v1 are independently 1 or 2;
X¹ is independently —F, —Cl, —Br, or —I; and
z1 is an integer from 0 to 6;
wherein the compound is not

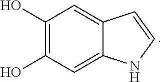 [SMILES: Oc1cc2cc[nH]c2cc1O]

3. (canceled)

4. The compound of claim 2, wherein R¹ is independently —F, —Cl, —Br, or —I.

5. The compound of claim 2, wherein the compound has the formula 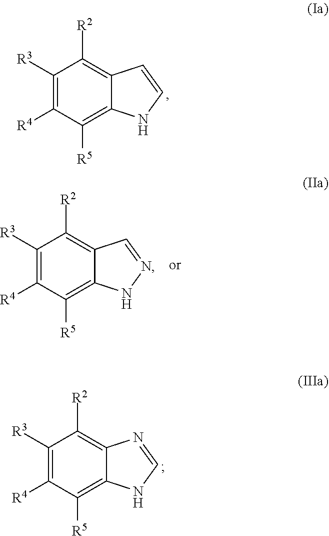
wherein
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, C(O)R^{2C}, —SC(O)R^{2C}, —C(O)OR^{2C}, —C(O)NR^{2A}R^{2B}, —OR^{2D}, —SR^{2D}, —SeR^{2D}, —NR^{2A}SO₂R^{2D}, —NR^{2A}C(O)R^{2C}, —NR^{2A}C(O)OR^{2C}, —NR^{2A}OR^{2C}, —N₃, —SF₅, —SSR^{2D}, —SiR^{2A}R^{2B}R^{2C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —SC(O)R^{3C}, —C(O)OR^{3C}, —C(O)NR^{3A}R^{3B}, —OR^{3D}, —SR^{3D}, —SeR^{3D}, —NR^{3A}SO₂R^{3D}, —NR^{3A}C(O)R^{3C}—NR^{3A}C(O)OR^{3C}, —NR^{3A}OR^{3C}, —N₃, —SF₅, —SSR^{3D}, —SiR^{3A}R^{3B}R^{3C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —SC(O)R^{4C}, —C(O)OR^{4C}, —C(O)NR^{4A}R^{4B}, —OR^{4D}, —SR^{4D}, —SeR^{4D}, —NR^{4A}SO₂R^{4D}, —NR^{4A}C(O)R^{4C}, —NR^{4A}C(O)OR^{4C}, —NR^{4A}OR^{4C}, —N₃, —SF₅, —SSR^{4D}, —SiR^{4A}R^{4B}R^{4C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —SC(O)R^{5C}, —C(O)OR^{5C}, —C(O)NR^{5A}R^{5B}, —OR^{5D}, —SR^{5D}, —SeR^{5D}, —NR^{5A}SO₂R^{5D}, —NR^{5A}C(O)R^{5C}, —NR^{5A}C(O)OR^{5C}, —NR^{5A}OR^{5C}, —N₃, —SF₅, —SSR^{5D}, —SiR^{5A}R^{5B}R^{5C}, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, and R^{5D} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may be joined to form a substituted or unsubstituted heterocycloalkyl or substituted or unsubstituted heteroaryl; R^{3A} and R^{3B} substituents bonded to the same nitrogen atom may be joined to form a substituted or unsubstituted heterocycloalkyl or substituted or unsubstituted heteroaryl; R^{4A} and R^{4B} substituents bonded to the same nitrogen atom may be joined to form a substituted or unsubstituted heterocycloalkyl or substituted or unsubstituted heteroaryl; R^{5A} and R^{5B} substituents bonded to the same nitrogen atom may be joined to form a substituted or unsubstituted heterocycloalkyl or substituted or unsubstituted heteroaryl;
n2, n3, n4, and n5 are independently an integer from 0 to 4;
m2, m3, m4, m5, v2, v3, v4, and v5 are independently 1 or 2; and
X², X³, X⁴, and X⁵ are independently —F, —Cl, —Br, or —I.

6. The compound of claim 5, wherein the compound has the

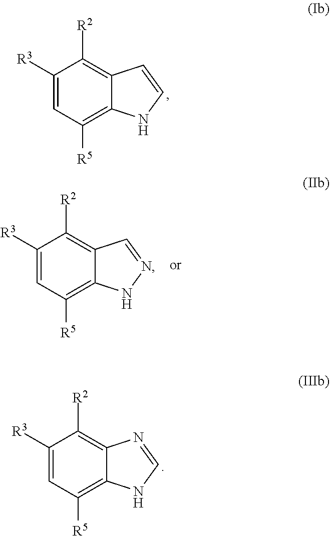

7. The compound of claim 5, wherein R³ is halogen.

8. The compound of claim 7, wherein R³ is —Br or —Cl.

9. (canceled)

10. The compound of claim 5, wherein R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

11. The compound of claim 5, wherein R² is hydrogen, halogen, —CF₃, —CH₂F, —CHF₂, —CN, —OH, —NH₂, —COOH, —CONH₂, —OCF₃, —OCHF₂, —OCH₂F, substituted or unsubstituted C₁-C₄ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

12. (canceled)

13. The compound of claim 5, wherein R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SeH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, —SF₅, —SP(O)(OH)₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

14. The compound of claim 5, wherein R⁵ is hydrogen, halogen, —CF₃, —CH₂F, —CHF₂, —CN, —OH, —NH₂, —COOH, —CONH₂, —OCF₃, —OCHF₂, —OCH₂F, substituted or unsubstituted C₁-C₄ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 10 membered heteroaryl.

15. A pharmaceutical composition comprising a compound of claim 2, or a pharmaceutically acceptable salt thereof, and a pharmaceutically acceptable excipient.

16. A method of treating a disease associated with dysregulation and/or degeneration of dopaminergic neurons in the central nervous system of a subject in need thereof, said method comprising administering to the subject in need thereof a therapeutically effective amount of a compound of claim 2, or a pharmaceutically acceptable salt thereof.

17. The method of claim 16, wherein said disease associated with dysregulation and/or degeneration of dopaminergic neurons is Parkinson's disease, Alzheimer's disease, multiple sclerosis, amyotrophic lateral sclerosis, schizophrenia, or drug addiction.

18. (canceled)

19. A method of treating a cancer in a subject in need thereof, the method comprising administering to the subject in need thereof a therapeutically effective amount of a compound of claim 2, or a pharmaceutically acceptable salt thereof.

20. The method of claim 19, wherein said cancer is breast cancer, pancreatic cancer, bladder cancer, mucoepidermoid carcinoma, gastric cancer, prostate cancer, colorectal cancer, lung cancer, adrenocortical cancer, or cervical cancer.

21. A method of modulating the level of activity of Nurr1 in a subject in need thereof, the method comprising administering to the subject in need thereof an effective amount of a compound of claim 2, or a pharmaceutically acceptable salt thereof.

22. A method of increasing the level and/or activity of Nurr1 in a cell, the method comprising contacting said cell with a compound of claim 2, or a pharmaceutically acceptable salt thereof.

23. A method of increasing the level of dopamine in a cell, the method comprising contacting said cell with a compound of claim 2, or a pharmaceutically acceptable salt thereof.

24. A method of differentiating a stem cell, the method comprising contacting said stem cell in vitro with a compound of claim 2, or a pharmaceutically acceptable salt thereof.

25. (canceled)
